(19) United States
(12) Patent Application Publication
(10) Pub. No.: US 2026/0258450 A1
(43) Pub. Date: Sep. 3, 2026

(54) RING SHAPED PLANT VIRAL NANOPARTICLE FOR GENE DELIVERY

(71) Applicant: The Regents of the University of California, Oakland, CA (US)

(72) Inventors: Nicole F. Steinmetz, San Diego, CA (US); Krister J. Barkovich, San Diego, CA (US); Bryan Duoto, Poway, CA (US)

(21) Appl. No.: 19/540,039

(22) Filed: Feb. 13, 2026

Related U.S. Application Data
(60) Provisional application No. 63/758,805, filed on Feb. 14, 2025.

(51) Int. Cl. C12N 15/88; C07K 14/005; C12N 15/85

(52) U.S. Cl. CPC C12N 15/88; C07K 14/005; C12N 15/85; C12N 2770/00022; C12N 2770/00023; C12N 2770/00051; C12N 2830/50; C12N 2840/203

(57) ABSTRACT

Provided herein are ring-shaped or halo-shaped nanoparticles comprising a filamentous plant virus coat protein, the coat protein being assembled on one or more circular RNA (circRNA) for use in diagnostic and therapeutic methods.

### CROSS-REFERENCE TO RELATED APPLICATION

This application claims the benefit under 35 U.S.C § 119(e) of U.S. Ser. No. 63/758,805, filed Feb. 14, 2025, the contents of which are incorporated herein by reference in their entirety.

### STATEMENT OF GOVERNMENT SUPPORT

This invention was made with government support under CA224605 awarded by the National Institutes of Health. The government has certain rights in the invention.

### BACKGROUND

Nucleic acid delivery plays important roles in basic research and medicine. The medical applications are wide ranging and touch on all aspects of medicine, including cancer, cardiovascular, infectious, and neurodegenerative diseases (Alzheimer's), as well as chronic diseases such as Parkinson's disease, cystic fibrosis, hemophilia. Gene delivery enables the delivery of therapeutic proteins or regulatory nucleic acids, for example small interfering RNAs (siRNAs) and microRNAs (miRNAs) to silence protein expression. Of note is that circRNAs have many functions and could be used for gene expression or gene regulation, for example they act as regulatory elements on miRNAs, transcription, translation, and even protein folding.

Beyond the clinical applications, nucleic acid delivery as a research tool has advanced a fundamental understanding of cell biology and physiology across kingdoms.

The success of circRNA therapy is contingent on overcoming significant challenges related to its inherent instability and the efficiency of its delivery to specific target cells.² This disclosure addresses these limitations and provides relevant advantages as well.

### SUMMARY OF THE DISCLOSURE

Applicant provides herein a new gene delivery platform that demonstrates the feasibility of using purified viral coat proteins (CPs) from the plant viruses to form virus-like particles (VLPs)—or nucleoprotein complexes with designer circular RNA (circRNA) through in vitro self-assembly. Applicant has observed that the transfection of these VLPs into eukaryotic cell lines allows for the disassembly of VLPs and the successful translation of the target proteins. Importantly, it was also established that the size of the resulting VLP are precisely controlled as a function of the length of the mRNA—resulting in designer nucleic acid delivery vehicles for tunable pharmacology.

The stabilizing features of mRNA, such as the 5′ 7-methyl guanosine (m7G) cap and the 3′ polyadenylated (poly(A)) tail, are fundamental in preserving the molecule from premature degradation. Similarly, circRNAs originate from pre-mRNA precursors that are spliced to form covalently closed-loop structures from the joining of the 5′ and 3′ ends of the RNA molecule—a feature that makes them less susceptible to exonucleases and increases their half-lives to up to seven times their linear counterparts. Yet, these features alone are insufficient for ensuring the stability and efficacy of RNA therapeutics within the biological milieu. Given that RNA is a negatively charged molecule, there is need to formulate RNA into carriers to screen the negative charge and enable uptake into cells (the mammalian cell membrane is also negative charged).

Applicant provides herein methods to produce nanoparticles (CPs) loaded with custom mRNA and circRNA and the application therefor. A modular system was developed that enabled size, shape, and functional control, the latter enabled through mixed assembly with functionalized CPs. In some aspects, CPs were obtained either from PVX farmed in Nicotiana benthamiana plants or recombinant expression in Escherichia coli. In another aspect, packaging of custom cargo was enabled through integration of the PVX OAS site (SL1). The resulting vectors' morphology and size were characterized through transmission electron microscopy (TEM) and size exclusion chromatography (SEC). Using a fluorescent reporter gene (enhanced green fluorescent protein, EGFP), gene delivery and protein expression was confirmed in mammalian cells using quantitative RT-PCR, flow cytometry, and imaging studies of cultured cell lines.

In some aspects, the disclosure provides a ring-shaped or halo-shaped nanoparticle including a filamentous plant virus coat protein, optionally wherein the filamentous plant virus coat protein is from Tobacco Mosaic Virus (TMV), Potato Virus X (PVX), Alternanthera mosaic virus, Papaya mosaic virus, or another member of the Potexvirus or Tobamovirus genera, the coat protein being assembled on one or more circular RNA (circRNA), optionally wherein the one or more circRNA are the same or different from each other.

In some aspects, the disclosure provides a polynucleotide including: (a) a RNA polymerase promoter; (b) a polynucleotide encoding a filamentous plant virus coat protein, optionally wherein the filamentous plant virus coat protein is from Tobacco Mosaic Virus (TMV), Potato Virus X (PVX), Alternanthera mosaic virus, Papaya mosaic virus, or another member of the Potexvirus or Tobamovirus genera, optionally a potato virus X (PVX) genome or a fragment thereof, wherein the polynucleotide includes an origin of assembly site sequence having a cis-acting element from the 5′ region of, and (c) a polyadenylation signal downstream of the polynucleotide encoding the plant virus coat protein or fragment. Examples of such are provided herein.

In some aspects, the disclosure provides an in vitro transcribed RNA including: (a) a 5′ cap structure including 7-methylguanylate; (b) a polynucleotide encoding a filamentous plant virus coat protein, optionally wherein the filamentous plant virus coat protein is from Tobacco Mosaic Virus (TMV), Potato Virus X (PVX), Alternanthera mosaic virus, Papaya mosaic virus, or another member of the Potexvirus or Tobamovirus genera, optionally a potato virus X (PVX) genome or a fragment thereof having an origin of assembly site sequence having a cis-acting element from the 5′ region of origin of assembly site sequence; and (c) a 3′ polyadenylate tail, wherein the polynucleotide encoding the filamentous plant genome or fragment has a predetermined length selected to control the size of a virus-like particle assembled from the RNA.

In some aspects, the disclosure provides a method of controlling the size of virus-like particles assembled from RNA derived from a plant virus genome, including generating RNA transcripts from a cloning vector that includes a promoter for in vitro transcription, an origin of assembly site sequence from said plant virus genome, and a polyadenylation signal, wherein the RNA transcript is capped and polyadenylated, and wherein the length of the plant virus genome sequence determines the particle size.

### BRIEF DESCRIPTION OF THE FIGURES

FIGS. 1A-1D: Representation of plasmids used for mRNA production, circRNA synthesis, and PVX CP protein fermentation. (FIG. 1A) Plasmid representation of pCMV-T7-PVX-Genome including a promoter for bacteriophage T7 RNA polymerase (T7 promoter), the entire genomic sequence for PVX's genome (PVX Genome), bovine growth hormone polyadenylation signal (BGH), high-copy-number origin of replication (ori), β-lactamase for resistance to ampicillin/carbenicillin (Amp^{R}), and fl bacteriophage origin of replication for (+) synthesis (fl ori). (FIG. 1B) Map pCMV-T7-SL1-EGFP with features consisting of a promoter for bacteriophage T7 RNA polymerase (T7 promoter), the PVX origin of assembly (SL1), codon-optimized enhanced GFP (EGFP), bovine growth hormone polyadenylation signal (BGH), high-copy-number origin of replication (ori), β-lactamase for resistance to ampicillin/carbenicillin (Amp^{R}), and human cytomegalovirus immediate early enhance (CMV enhancer) and promoter (CMV promoter). (FIG. 1C) Vector map of Circ-EMCV-EGFP-SL1 including a promoter for bacteriophage T7 RNA polymerase (T7 promoter), flanking twister ribozymes (Twister-P3; Twister P1) that rapidly self-cleave and allow for hybridization of ligation stems, exon-intron sequences (Exon-Intron 1; Exon-Intron 2) that allow for autocatalytic intron splicing, internal ribosomal entry site (IRES), codon-optimized enhanced GFP (EGFP), PVX origin of assembly (SL1), woodchuck hepatitis virus posttranscriptional regulatory element (WPRE), Poly(A), and β-lactamase for resistance to ampicillin/carbenicillin (AmpR). (FIG. 1D) Plasmid map of pHGWA-9×His-PVX-CP featuring a promoter for bacteriophage T7 RNA polymerase (T7 promoter), a 9× His affinity tag (9×His), TEV protease cleavage site (TEV Site), PVX CP, transcription terminator for bacteriophage T7 RNA polymerase (T7 Term), fl bacteriphage origin of replication for (+) synthesis (fl ori), β-lactamase for resistance to ampicillin/carbenicillin (AmpR), high-copy-number origin of replication (ori), lac repressor (lacl) and its promoter (lacl promoter) for repressing transcription, and tetracycline efflux protein (TcR). Created with BioRender. (Tables 2 and 3) for details on the gBlocks and plasmid sequences.

FIG. 2: Representation of in vitro assembly process for PVX VLPs and in vitro transcription of mRNA transcripts using the EGFP-SL1 payload as an example. Created with BioRender.

FIGS. 3A-3E. Size control of PVX VLPs through templated assembly of CP on genomic RNA (gRNA) fragments. (FIG. 3A) Graphical representation of genomic mRNA fragment design and VLP production. Created with BioRender. (FIG. 3B) Agarose gel electrophoresis of PCR products (upper) and gRNA fragments after m7G capping, poly(A) tailing, and purification (lower). Uncropped images are provided in FIG. 7. (FIG. 3C) TEM images of representative VLPs, CPs, and PVX. (FIG. 3D) Histograms normalized to most frequent VLP sizes per gRNA length as compared to the negative control (PVX CP) and positive control (WT PVX); the 6.4-knt assembly is colored red to show a significant size distribution shift and to highlight which VLP condition exhibits the largest size shift. (FIG. 3E) Size distributions of measured particles from TEM images across different size conditions with density line overlays; colored as a spectrum ranging from smallest (250 nt, purple) to largest conditions (6.4 knt, yellow). VLP lengths (nm) were normalized to each dataset's maximum value to compare relative distribution shapes across conditions with different input RNA sizes. Histograms show the normalized frequency of particle lengths, and Kernal Density Estimation (KDE) curves provide smoothed density estimates). KDE is a non-parametric method for estimating the probability density function of a dataset by summing smooth kernel functions (e.g., Gaussian) centered at each observation, producing a continuous curve that represents the underlying distribution.

FIGS. 4A-4E. VLPs were obtained through mixing defined ratios of recombinant rCP-His and wildtype CPs. (FIG. 4A) Representative cartoon of the mechanisms for 10 nm Ni-NTA-Nanogold® conjugation to the N-terminal 9×His tags on recombinant PVX CPs derived from E. coli fermentation (created using Biorender). (FIG. 4B) 4-12% SDS-Page (upper) and Goldiblot™ western (lower) of mixed assembly VLPs organized by CP ratios. Uncropped gels and blots are shown in FIG. 8C. (FIG. 4C) Representative TEM images of Au 10 nm Ni-NTA-Nanogold®-conjugated mixed assembly VLPs. (FIG. 4D) Measured frequencies, as measured by the number of Au particles bound to VLPs organized by mixed assembly ratio condition. (FIG. 4E) Pairwise statistical comparison of bound Au particle frequencies using the Mann-Whitney U test. The heatmap displays the p-values from nonparametric comparisons between all groups, with lower p-values (lighter cells) indicating greater statistical significance in Au nanoparticle-VLP conjugation frequencies by ratio combination.

FIGS. 5A-5I. Design and Analysis of EGFP-SL1 VLPs. (FIG. 5A) Schematic representation of the mRNA cassette containing EGFP-SL1. EGFP-SL1 mRNA was constructed by encoding the OAS site of the PVX genome, stem-loop 1 (SL1), upstream of the EGFP reporter sequence, flanked by the untranslated regions at both the 5′ and 3′ terminus, and joined with an m7G cap at the 5′ terminal end and a Poly(A) tail at the 3′ terminal end. (FIG. 5B) mRNA transcripts generated by in vitro transcription (IVT) and purified by LiCl precipitation were quantified and analyzed in comparison with the heat-denatured Agilent RNA 6000 ladder using the Agilent 2100 bioanalyzer system. (FIG. 5C) Size-exclusion chromatograms (SEC) of PVX CP control (top), native PVX (middle) and VLPs assembled with EGFP-SL1 mRNA (bottom). The size distribution of measured particles of in vitro assembled EGFP-SL1 VLPs (blue and black) as compared to VLPs made with PVX CP (orange) and PVX gRNA (orange and black). (FIG. 5D) Length measurements from TEM measurements (upper) and sample 260 nm:280 nm ratios of EGFP-SL1 VLPs as compared to PVX and PVX CP (lower). (FIG. 5E) TEM micrographs of VLPs made with EGFP-SL1 (upper), gRNA (middle), and wildtype PVX (lower). (FIG. 5F) Confocal microscopy of BHK-21 cells transfected with different conditions of VLPs and mRNA (listed); scale bar represents 400 μm. (FIG. 5G) Transfection efficiencies of the corresponding conditions as measured by % EGFP+ cells. (FIG. 5H) Distribution of mass of VLPs, in μg, per million viable cells in triplicate. (FIG. 5I) RT-PCR showing relative RNA levels of free EGFP-SL1 mRNA, disassembled EGFP-SL1 VLPs (100-300 μg), free CP, and disassembled PVX virus in relation to original EGFP mRNA (no SL1). Error bars represent standard deviation. Statistical significance was calculated through two-way ANOVA; *** p<0.0001, ** p<0.005, * p<0.05, NS: not significant. Abbreviations: L2K=Lipofectamine 2,000, EGFP=enhanced green fluorescent protein, PVX=Potato Virus X, CP=coat protein, and VLP=virus-like particle.

FIGS. 6A-6G. Design, characterization, and analysis of EGFP-SL1 circRNA VLPs. (FIG. 6A) Schematic representation of circRNA genetic components, IVT reaction, and circularization mechanism. (FIG. 6B) pre-circRNA and circRNA transcripts, as well as free introns, generated by in vitro transcription and purified by LiCl precipitation were analyzed in comparison with the heat-denatured Agilent RNA 6000 ladder before and after the addition of RNAseR. (FIG. 6C) Size-exclusion chromatogram of EGFP-SL1 circVLPs as overlayed 260 nm and 280 nm distributions. (FIG. 6D) Size measurements of circVLPs and circRNA from TEM micrographs. (FIG. 6E) TEM micrographs of EGFP-SL1 circRNA, partial circVLP, and full circVLP. (FIG. 6F) Transfection delivery efficiencies, as measured by flow cytometry, for EGFP+ HEK 293T cells expressing payloads from circVLPs and circRNA controls. (FIG. 6G) Confocal microscopy images of transfected HEK 293T cells across the different timepoints and conditions.

FIGS. 7A-7B. Uncropped gel images as shown in FIG. 3B. (FIG. 7A) Uncropped 1.2% (w/v) agarose gel in TBE buffer of PCR products used for generating mRNA transcripts. (FIG. 7B) Uncropped 1.2% (w/v) agarose gel in TBE buffer of mRNA transcripts derived from PCR products via in vitro transcription.

FIGS. 8A-8E. Information on mixed assembly VLPs. (FIG. 8A) Yields of N-terminal 9× His-tagged CPs after purification from (DE3) BL21 cells. (FIG. 8B) Size exclusion chromatography (SEC) of the different mixed assembly VLP conditions after assembly and purification for ratio combinations that did not reliably produce VLPs; an example of mixed assembly using rCP-His with C-terminal His tag and native CP is also included to showcase lack of assembly—the same condition yielded VLPs when rCP-His with N-terminal His-tag was used. (FIG. 8C) Uncropped Goldiblot Western gel with ladder displaying VLPs at a characteristic molecular weight of ~28 kDa; see also FIG. 4B. (FIG. 8D) Purification of rCP-His from bacterial pellets including lanes for the flow-through (FT), wash (W), and elution (FIG. 8E) samples in a 4-12% Bis-Tris SDS-PAGE gel. (FIG. 8E) Purified C-terminal rCP-His (R) was also derived from bacterial cultures and exhibited >90% purity as compared to BSA, however C-terminal rCP-His did not lead to successful VLP formation in assembly conditions tested (see FIG. 8B).

FIGS. 9A-9C. Information on circVLPs and the design rationale of circRNA. (FIG. 9A) Three different locations were chosen for insertion of the SL-1 OAS site to study the effects of the RNA stem loop on circularization and expression of the reporter gene. (FIG. 9B) After transfection of circRNAs into HEK 293T cells using Lipofectamine 2,000, delivery efficiencies were observed across the three locations with the post-EGFP SL1 working the best. (FIG. 9C) The circRNAs from the post-EGFP SL1 condition were shown to successfully circularize and were purified away from linear pre-circRNA transcripts and free introns.

FIG. 10: Structure of potato virus X (PVX) (left) and negative-stained TEM image of PVX (right).

FIG. 11: Size exclusion chromatography of intact TMV (black), its coat protein (right peak), and a 60 nm-sized reassembled VLP (left peak). The methods are exemplary for PVX.

FIGS. 12A-12D: (FIG. 12A) The atomic model of PVX. Cys and Lys residues are highlighted in red and green, respectively. (FIG. 12B) The PVX structure model showing a helical structure. (FIG. 12C) Top view and (FIG. 12D) side view of a short segment of PVX displaying surface-exposed Cys and Lys. The location of the RNA is highlighted as orange circle.

FIGS. 13A-13D: Mechanical performance of VLPs by single-molecule force spectroscopy (SMSF). (FIG. 13A) Schematic. (FIG. 13B) Representative force-extension curve of PhMV and crosslinked EE-PhMV1000. (FIGS. 13C-13D) Statistic mapping shows the two-dimensional distribution of rupture forces and distance of PhMV and EE-PhMV1000. The rupture distance Drup and rupture force Frup are 6-fold and 1.9-fold increased for the crosslinked EE-PhMV1000 sample (p<0.001, n=71).

FIG. 14: RNA-dependent RNA polymerase (RdRp) amplification of a gene of interest (GOI). Translation from start (<) to stop (>) codons in an mRNA construct, made from the RdRp gene of a positive-sense RNA viral genome to which has been added a gene of interest separated by self-cleaving T2A peptide sequence, results in replication of the construct by the RdRp

FIG. 15: PVX vs. contemporary gene delivery systems.

FIG. 16: PVX cell uptake in RAW247.6 cells comparing PEG vs. RGD-conjugated PVX; after 24 hours, there was no significant co-localization between PVX (green) and endolysomal marker (Lamp-1).

FIG. 17: PVX vs. contemporary gene delivery systems.

FIG. 18: Plasmid map of pCMV-TV-SL1-EGFP-BGH.

FIG. 19: Plasmid map of Circ-EMCV-GFP-SL1.

FIG. 20: Plasmid map of pCMV-T7-PVX-Genome.

FIG. 21: Plasmid map of Phgwa-9×His-PVX-CP.

### DETAILED DESCRIPTION

### Definitions

As used in the specification and claims, the singular form “a”, “an”, and “the” include plural references unless the context clearly dictates otherwise. For example, the term “a cell” includes a plurality of cells, including mixtures thereof.

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure. For example, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions disclosed herein. Aspects defined by each of these transition terms are within the scope of the present disclosure.

As used herein, the term “about” is used to indicate that a value includes the standard deviation of error for the device or method being employed to determine the value. The term “about” when used before a numerical designation, e.g., temperature, time, amount, and concentration, including range, indicates approximations which can vary by (+) or (−) 15%, 10%, 5%, 3%, 2%, or 1%.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 m in diameter and 10 m long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

A “composition” typically intends a combination of the active agent, e.g., the ring-shaped nanoparticle of this disclosure and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

As used herein, the terms “nucleic acid sequence,” “oligonucleotide,” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

The term “encode” as it is applied to nucleic acid sequences refers to a polynucleotide which is the to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “isolated cell” generally refers to a cell that is substantially separated from other cells of a tissue. The term includes prokaryotic and eukaryotic cells.

“Immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Cytokines are small secreted proteins released by immune cells that have a specific effect on the interactions and communications between the immune cells. Cytokines can be pro-inflammatory or anti-inflammatory. Non-limiting example of a cytokine is Granulocyte-macrophage colony-stimulating factor (GM-CSF), which stimulates stem cells to produce granulocytes (neutrophils, eosinophils, and basophils) and monocytes.

As used herein, the term “vector” refers to a nucleic acid construct deigned for transfer between different hosts, including but not limited to a plasmid, a virus, a cosmid, a phage, a BAC, a YAC, etc. A “viral vector” is defined as a recombinantly produced virus or viral particle that comprises a polynucleotide to be delivered into a host cell, either in vivo, ex vivo or in vitro. In some embodiments, plasmid vectors can be prepared from commercially available vectors. In other embodiments, viral vectors can be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. Further details as to modern methods of vectors for use in gene transfer can be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif) and Promega Biotech (Madison, Wis.).

As used herein, “nanoparticle and “VLP” both refer to a viral vector comprised of a virus coat protein and a nucleic acid. More particularly, the ring-shaped nanoparticle refers to a viral vector comprised of a virus coat protein, preferably PVX, and circRNA.

An “effective amount” or “efficacious amount” refers to the amount of an agent or combined amounts of two or more agents, that, when administered for the treatment of a mammal or other subject, is sufficient to cause such treatment for the disease. The “effective amount” will vary depending on the agent(s), the disease and its severity and the age, weight, etc., of the subject to be treated.

As used herein, a “cancer” is a disease state characterized by the presence in a subject of cells demonstrating abnormal uncontrolled replication and can be used interchangeably with the term “tumor.”

The tumor is not limited and can be any kind of cancer, e.g., solid or blood cancer, e.g., carcinoma or sarcoma. In some embodiments, the cancer is ICI resistant. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

A “solid tumor” is an abnormal mass of tissue that usually does not contain cysts or liquid areas. Solid tumors can be benign or malignant. Different types of solid tumors are named for the type of cells that form them. Examples of solid tumors include sarcomas, carcinomas, and lymphomas. In some embodiments, a solid tumor comprises bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, prostate cancer, or stomach cancer.

As used herein, the term “hematologic malignancy” refers to cancers with hematopoietic origin. In some instances, the hematologic malignancy is a B-cell malignancy. In some instances, the hematologic malignancy is a lymphoma, optionally a B-cell lymphoma. Exemplary hematologic malignancies include, but are not limited to, Diffuse large B-cell lymphoma (DLBCL), follicular lymphoma, chronic lymphocytic leukemia (CLL), small lymphocytic lymphoma (SLL), mantel cell lymphoma (MCL), marginal zone lymphomas, Burkitt lymphoma, Waldenstrom macroglobulinemia, hairy cell leukemia (HCL), primary central nervous system (CNS) lymphoma, or primary intraocular lymphoma.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection can be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I.

As used herein, the term “purification marker” or “reporter protein” refer to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of a compound.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding the PVX described herein). Homology can be determined by comparing a position in each sequence which can be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical,” percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

It is to be inferred without explicit recitation and unless otherwise intended, that when the present disclosure relates to a polypeptide, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide, or nucleic acid, intends those having minimal homology while still maintaining desired structure or functionality. Unless specifically recited herein, it is contemplated that any of the above also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and/or exhibits substantially equivalent biological activity to the reference protein, polypeptide, or nucleic acid. Alternatively, when referring to polynucleotides, an equivalent thereof is a polynucleotide that hybridizes under stringent conditions to the reference polynucleotide or its complement.

The phrase “equivalent polypeptide” or “equivalent peptide fragment” refers to protein, polynucleotide, or peptide fragment encoded by a polynucleotide that hybridizes to a polynucleotide encoding the exemplified polypeptide or its complement of the polynucleotide encoding the exemplified polypeptide, under high stringency and/or which exhibit similar biological activity in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding can occur by Watson-Crick base pairing, Hoogstein binding, or in any other sequence-specific manner. The complex can comprise two strands forming a duplex structure, three or more strands forming a multi-stranded complex, a single self-hybridizing strand, or any combination of these. A hybridization reaction can constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. A high stringency hybridization refers to a condition in which hybridization of an oligonucleotide to a target sequence comprises no mismatches (or perfect complementarity). Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “protein”, “peptide” and “polypeptide” are used interchangeably and in their broadest sense to refer to a compound of two or more subunit amino acids, amino acid analogs or peptidomimetics. The subunits can be linked by peptide bonds. In another aspect, the subunit can be linked by other bonds, e.g., ester, ether, etc. A protein or peptide must contain at least two amino acids and no limitation is placed on the maximum number of amino acids which can comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation can be essentially homogeneous, wherein other macromolecular species are not detectable by conventional techniques.

As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

The terms “fusion” or “chimeric” and grammatical variations thereof, when used in reference to a molecule, such as a PVX, means that a portions or part of the molecule contains a different entity distinct (heterologous) from the molecule as they do not typically exist together in nature. That is, for example, one portion of the fusion or chimera, such as PVX, includes or consists of a portion that does not exist together in nature, and is structurally distinct.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

As used herein, the term “overexpress” with respect to a cell, a tissue, or an organ expresses a protein to an amount that is greater than the amount that is produced in a control cell, a control issue, or an organ. A protein that is overexpressed can be endogenous to the host cell or exogenous to the host cell.

As used herein, the term “enhancer”, denotes sequence elements that augment, improve or ameliorate transcription of a nucleic acid sequence irrespective of its location and orientation in relation to the nucleic acid sequence to be expressed. An enhancer can enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

The term “promoter” as used herein refers to any sequence that regulates the expression of a coding sequence, such as a gene. Promoters can be constitutive, inducible, repressible, or tissue-specific, for example. A “promoter” is a control sequence that is a region of a polynucleotide sequence at which initiation and rate of transcription are controlled. It can contain genetic elements at which regulatory proteins and molecules can bind such as RNA polymerase and other transcription factors.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

The term “introduce” as applied to methods of producing modified cells such as chimeric antigen receptor cells refers to the process whereby a foreign (i.e. extrinsic or extracellular) agent is introduced into a host cell thereby producing a cell comprising the foreign agent. Methods of introducing nucleic acids include but are not limited to transduction, retroviral gene transfer, transfection, electroporation, transformation, viral infection, and other recombinant DNA techniques known in the art. In some embodiments, transduction is done via a vector (e.g., a viral vector). In some embodiments, transfection is done via a chemical carrier, DNA/liposome complex, or micelle (e.g., Lipofectamine (Invitrogen)). In some embodiments, viral infection is done via infecting the cells with a viral particle comprising the polynucleotide of interest (e.g., AAV). In some embodiments, introduction further comprises CRISPR mediated gene editing or Transcription activator-like effector nuclease (TALEN) mediated gene editing. Methods of introducing non-nucleic acid foreign agents (e.g., soluble factors, cytokines, proteins, peptides, enzymes, growth factors, signaling molecules, small molecule inhibitors) include but are not limited to culturing the cells in the presence of the foreign agent, contacting the cells with the agent, contacting the cells with a composition comprising the agent and an excipient, and contacting the cells with vesicles or viral particles comprising the agent.

The term “culturing” refers to growing cells in a culture medium under conditions that favor expansion and proliferation of the cell. The term “culture medium” or “medium” is recognized in the art and refers generally to any substance or preparation used for the cultivation of living cells. The term “medium”, as used in reference to a cell culture, includes the components of the environment surrounding the cells. Media can be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture can be termed a “powdered medium.” “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high-density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium can be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (IMDM).

“Cryoprotectants” are known in the art and include without limitation, e.g., sucrose, trehalose, and glycerol. A cryoprotectant exhibiting low toxicity in biological systems is generally used.

A non-coding RNA (ncRNA) is an RNA molecule that is not translated into a protein. Non-limiting examples of non-coding RNA include transfer RNAs (tRNAs) and ribosomal RNAs (rRNAs), microRNAs, siRNAs etc.

Circular RNA (circRNA) intends a type of single-stranded RNA which, unlike linear RNA, forms a covalently closed continuous loop. In circular RNA, the 3′ and 5′ ends normally present in an RNA molecule have been joined together. This feature confers numerous properties to circular RNA, many of which have only recently been identified. Studies have identified three main types of circRNA: ecircRN, circular intronic RNA (ciRNA), and exon-intron circRNA (EIciRNA).

As used herein, the term “Tobacco Mosaic Virus” or “TMV” refers to a member of the genus Tobamovirus comprising a single-stranded RNA genome encapsidated by coat proteins forming a rigid rod-shaped structure approximately 300 nanometers in length and 18 nanometers in diameter. TMV coat protein can be genetically or chemically modified and used for RNA encapsulation into virus-like particles as disclosed herein. The term “filamentous plant virus” includes TMV, Potato Virus X (PVX), and additional members of the Potexvirus or Tobamovirus genera and other filamentous plant viruses such as Alternanthera mosaic virus, Papaya mosaic virus, Cucumber green mottle mosaic virus, and Pepper mild mottle virus, which may substitute for PVX or TMV in the compositions, methods, and assemblies described.

The term “targeting moiety” intends a molecule or molecular complex that binds selectively to a site, receptor, or structure on a target cell to increase uptake or localization of a nanoparticle. Targeting moieties include peptides, antibodies or antibody fragments, aptamers, carbohydrates, ligands for cell-surface receptors, and synthetic binding scaffolds. Targeting moieties can be covalently linked, non-covalently complexed, or genetically fused to a coat protein and may be designed to direct uptake to specific tissues or tumors.

The term “self-amplifying RNA” intends an RNA molecule encoding its own RNA-dependent RNA polymerase or equivalent viral replicase, which can replicate the RNA in the cytoplasm of a target cell without integration into the genome. Self-amplifying RNAs contain a gene of interest separated from the RNA-dependent RNA polymerase sequence by a self-cleaving peptide, allowing co-expression of replicase and gene of interest, with the replicase amplifying the overall replicon and its gene products. Replicons derived from alphaviruses including Sindbis virus and Venezuelan equine encephalitis virus, and from nodaviruses, are non-limiting examples.

The term “crosslinker” intends a bifunctional or multifunctional chemical reagent capable of covalently joining two or more coat protein subunits. Crosslinkers can be homobifunctional or heterobifunctional and may target lysine, cysteine, histidine, or other reactive residues. Crosslinkers can be linear or branched. A “PEG crosslinker” intends a crosslinker comprising a poly(ethylene glycol) chain of any molecular weight, optionally functionalized at each terminus with reactive groups targeting lysine or cysteine residues such as an NHS ester or maleimide group.

The term “reactive residue” intends any amino acid side chain that can be chemically modified under suitable conditions and includes lysine, cysteine, histidine, and other residues with reactive amine, thiol, imidazole, carboxylic acid, or other functional groups. In the PVX coat protein and other plant viral coat proteins, naturally occurring lysine and cysteine residues are solvent-exposed and amenable to chemical conjugation.

The term “rupture force,” abbreviated Frup, intends the maximum force applied to a nanoparticle structure before mechanical failure or breakage, measured in piconewtons by single-molecule force spectroscopy. The term “rupture distance,” abbreviated Drup, intends the extension length a nanoparticle structure undergoes before mechanical failure, measured in nanometers by single-molecule force spectroscopy. “Single-molecule force spectroscopy” intends an atomic force microscopy-based technique that applies and measures force-extension curves for individual nanostructures to determine mechanical strength and elasticity.

The term “endocytosis pathway inhibitors” intends chemical compounds that selectively block cellular uptake pathways, including clathrin-mediated endocytosis, caveolin-mediated endocytosis, and macropinocytosis. Non-limiting examples of such inhibitors include chlorpromazine, filipin, and amiloride, which may be used experimentally to evaluate mechanisms of nanoparticle entry into cells.

The term “halo-shaped nanoparticle” intends a circular or ring-like architecture observed in transmission electron microscopy for nanoparticles assembled around a circular RNA template. Halo-shaped particles can have continuous or partial coat protein coverage around the RNA loop and may also be referred to as ring-shaped nanoparticles.

The term “PEGylation” intends the process of covalently attaching poly(ethylene glycol) molecules to a nanoparticle to improve solubility, reduce immunogenicity, and modify biodistribution in vivo or in vitro. The term “RGD modification” intends covalent or genetic incorporation of an RGD peptide motif onto a coat protein to target integrin receptors for enhanced cellular uptake.

The term “GM-CSF bioassay” intends an assay for granulocyte-macrophage colony-stimulating factor activity, typically based on proliferation or survival of GM-CSF-dependent cell lines such as TF-1 cells. The term “IL-2 bioassay” intends an assay for interleukin-2 activity, typically based on proliferation or activation markers in IL-2-responsive cell lines such as CTLL-2 cells.

As used herein, the term “diagnostic moiety” refers to any component, agent, or molecular feature that enables the detection, monitoring, or imaging of the nanoparticle, biological tissues, or physiological processes for diagnostic purposes. A diagnostic moiety may directly produce a detectable signal, or may generate a signal indirectly after an interaction or reaction with a detectable substrate. Non-limiting examples include radiotracers (e.g., ^{18F-FDG}, 64Cu-ATSM), MRI contrast agents (e.g., gadolinium chelates, iron oxide nanoparticles), ultrasound contrast agents (e.g., microbubbles), positron emission tomography (PET) agents, computed tomography tracers (e.g., iodinated compounds), optical probes (e.g., fluorescent dyes, luminescent reporters such as quantum dots), and detectable haptens or epitopes that can be visualized using labeled antibodies. In some embodiments, the diagnostic moiety is identical to, or comprises, a “detectable marker” as defined herein.

As used herein, the term “therapeutic moiety” refers to any agent, compound, or molecular construct that provides a therapeutic effect to a subject when delivered by the nanoparticle. Therapeutic moieties include, but are not limited to, small-molecule drugs, peptides, proteins, nucleic acids (e.g., siRNA, antisense oligonucleotides, therapeutic mRNA or circRNA), gene editing systems (e.g., CRISPR/Cas components), cytokines, chemokines, antibodies or antibody fragments, immunomodulatory ligands, prodrugs, and toxins. In certain embodiments, the therapeutic moiety is conjugated to the coat protein via chemical modification of a reactive residue, incorporated genetically as a fusion, or encapsulated within the nanoparticle's interior. Therapeutic moieties may be used alone or in combination with other agents to achieve additive or synergistic therapeutic effects.

As used herein, the term “self-cleaving peptide sequence” refers to a polypeptide segment that, when expressed as part of a larger polyprotein, undergoes autocatalytic cleavage or ribosome-mediated “stop-carry on” translation (ribosomal skipping) to generate two or more separate protein products. Such self-cleaving sequences facilitate co-expression of multiple proteins from a single open reading frame without requiring multiple promoters or transcription units. Non-limiting examples include 2A peptide sequences derived from picornaviruses such as Thosea asigna virus 2A (T2A), Porcine teschovirus-1 2A (P2A), and Equine rhinitis A virus 2A (E2A), as well as F2A and other viral 2A-like sequences known in the art.

As used herein, the term “circumference” refers to the measured length of the closed outer boundary of a generally circular or ring-shaped nanoparticle, as visualized for example by transmission electron microscopy (TEM) or atomic force microscopy (AFM). Circumference may be calculated directly from TEM images using pixel-to-nanometer conversion factors, or indirectly from measured particle diameter using the formula C=π×D, where C is circumference in nanometers and Dis the diameter in nanometers. In some embodiments, the circumference corresponds to the contour length around the particle's exterior surface.

As used herein, the term “ring-shaped nanoparticle” refers to a halo-shaped nanoparticle as defined herein, having a circular or annular architecture observable in micrographs such as TEM. For the purposes of this disclosure, “ring-shaped nanoparticle” and “halo-shaped nanoparticle” are synonymous and both refer to viral coat protein assemblies around a circular RNA template resulting in a continuous or partial closed loop morphology.

As used herein, the term “membrane-active, pH-sensitive fusogenic peptide” refers to a peptide that undergoes conformational change under acidic pH conditions, such as those present in endosomes or lysosomes, enabling the peptide to interact with and destabilize lipid bilayers to promote release of the nanoparticle cargo into the cytosol. Non-limiting examples include the GALA peptide (WEAALAEALAEALAEHLAEALAEALEALAA), influenza virus hemagglutinin HA2 fusion peptide, and pH-Low Insertion Peptide (pHLIP) variants. Such peptides may be covalently conjugated or genetically fused to the coat protein, and function by enhancing endosomal escape of circRNA payloads after nanoparticle uptake by a cell.

### Modes for Carrying Out the Disclosure

Advancements in protein-based nanoparticle technology have provided novel solutions for the encapsulation, protection, and targeted delivery of RNA molecules, including mRNA and circRNA.⁴ As described herein, the primary RNA of interest is circRNA. The stabilizing features of mRNA, such as the 5′ 7-methyl guanosine (m7g) cap and the 3′ polyadenylated (poly(a)) tail, are fundamental in preserving the molecule from degradation. Yet, these features alone are insufficient for ensuring the stability and efficacy of mRNA therapeutics within the biological milieu. To this end, the integration of RNA-protein binding sites, mimicking interactions found in nature, alongside the utilization of coat proteins (CPs) derived from plant viruses, has been identified as a promising strategy.⁵ This approach leverages the evolutionary adaptations of viruses to protect and deliver their genetic material, and several proof-of-concept studies have successfully evaluated the potential of plant viruses, and their constituent VLPs, to mechanistically encapsulate foreign RNAs and deliver them to mammalian cells.^{6,7}

Potato Virus X (PVX), a plant virus belonging to the Potexvirus genus of the family Flexiviridae, is an exemplary candidate for such applications. The genomic RNA (gRNA) of PVX shares critical features with mammalian mRNA, including the m7g cap and poly(a) tail, facilitating its assembly into VLPs that can serve as effective RNA (for example, mRNA or circRNA) delivery vehicles.⁸ The assembly process of PVX is characterized by a highly conserved nucleation mechanism, initiating the formation of a stable core structure around its viral RNA.⁹ This core acts as a foundation for the sequential recruitment and assembly of CP subunits, driven by interactions with specific RNA sequences and structural motifs within the PVX genome.⁹ The AC-rich single-stranded sequence and the stem-loop structure at the 5′ region of the PVX genome, called stem-loop 1 (SL1), form the origin of assembly (OAS) site and play pivotal roles in this process, enabling the specific recognition and binding of CP subunits, and highlighting the intricate assembly mechanism that could be harnessed for therapeutic mRNA delivery.⁵

The physical and structural characteristics of PVX provide further evidence for its suitability as a nanotechnology platform. PVX virions are described as flexible, thread-like bodies measuring approximately 500-515 nanometers in length and 13-15 nanometers in diameter.¹⁰ Each viral particle comprises around 1300-1350 helically folded identical CP subunits enclosing a 6.4 knt viral RNA, with each turn of the primary helix consisting of 8.9 CP subunits—ideal traits for large nucleic acid payloads.¹⁰ This capability, coupled with the potential for genetic and chemical modifications of the virus, opens new avenues for the development of bespoke nanocarriers tailored to specific therapeutic applications and payloads.

Despite the success of synthetic nanocarriers, such as lipid nanoparticles (LNPs) in the delivery of mRNA vaccines, there remains significant scope for innovation and improvement.¹¹ A PVX-based delivery platform offers a novel alternative, promising to surmount the limitations of current manufacturing systems through enhanced scalability, cost-effectiveness, and environmental sustainability.¹² By implementing molecular farming, fermentation, and cell-free synthesis, the production of RNA and PVX CP are optimized for distributed manufacturing models closer to treatment sites.^{12,13} This strategy has the potential to alleviate supply chain bottlenecks, reduce production costs, and facilitate the global accessibility of RNA-based therapeutics and vaccines.^{12,13}

Plant virus VLPs, such as those derived from PVX, present distinct advantages, including remarkable stability, efficacy under physiological conditions, and broad biocompatibility, making them highly attractive for both vaccine development and gene therapy.^{4,14} The immunostimulatory properties of VLPs, serving dual roles as delivery vehicles and adjuvants, enhance the immunogenicity and therapeutic efficacy of the encapsulated mRNA.^{15,16} Their repetitive and highly organized structures act as targets for pathogen-associated molecular patterns (PAMPs), which are recognized by pattern-recognition receptors (PRRs), most notably toll-like receptors (TLRs)—key modulators of the body's innate immune responses.¹⁷ The majority of capsids and viral coat proteins are recognized by TLR-2 (in addition to other PRRs), however, VLPs containing RNA also stimulate TLR7/8.¹⁸ Furthermore, the unique physicochemical properties of PVX VLPs, including their filamentous structure and high-aspect ratio, enable specific interactions with the immune system, promoting targeted delivery and potent immune responses.¹⁹ The capacity of PVX-based systems to leverage the Enhanced Permeability and Retention (EPR), a phenomenon attributed to the passive targeting of nanoparticles to tumors due to leaky vasculature and poor lymphatic drainage, combined with their demonstrated affinity for malignant B cells, exemplifies the precision and versatility of this platform for targeted therapeutic applications.¹⁹

To this end, Applicant set out to develop a VLP-based gene delivery strategy for the delivery of RNA to cells in vitro. More specifically, Applicant developed a platform for the molecular farming of infected Nicotiana Benthamiana plants to isolate PVX virions, and later CP subunits, for in vitro reconstitution of VLPs from PVX CP in combination with an mRNA containing the PVX OAS site (SL1) and a fluorescent reporter gene (EGFP). The resulting nucleoprotein assemblies form high-aspect-ratio filamentous particles with helical symmetry, moderate monodispersity, and morphology resembling the original PVX virions. PVX VLPs reconstituted with the EGFP-SL1 mRNA had median measurements of 40-375 nm in length and 13-15 nm in diameter, while VLPs reconstituted with PVX gRNA measured 250-725 nm in length and 13-15 nm in diameter, demonstrating a trend in size control and higher polydispersity when reconstituted as VLPs. VLP morphology and size measurements were characterized through transmission electron microscopy (TEM) and size exclusion chromatography (FPLC). VLP delivery efficiency and EGFP protein expression in vitro were determined through flow cytometry, quantitative RT-PCR, and imaging studies of cultured cell lines. Successful delivery of the VLPs was carried out using commercially available lipid nanoparticles (Lipofectamine™ 2000) to ascertain the ability of VLPs to effectively protect mRNA payloads and disassemble within the cytosol of target cells.

Further, Applicant sets out to develop a platform molecular farming of infected Nicotiana Benthamiana plants to isolate PVX virions, and later coat protein subunits, for in vitro reconstitution of VLPs from PVX CP in combination with a circRNA. Without wishing to be bound by any particular theory, Applicant theorizes that the resulting nucleoprotein assemblies will form high-aspect-ratio filamentous particles with helical symmetry, moderate monodispersity, and a ring-shaped morphology.

Table 1 shows a comparison of the different viral nanoparticle systems. Of particular interest described herein are ring-shaped nanoparticles comprised of circRNA. The nanoparticles are incorporated into compositions and used in methods to deliver circRNA to cells and treat cancer.

TABLE 1
Pros (bold) and cons (italics) different nanoparticles for nucleic acid delivery.

| | Viral vector | Non-viral vector | Plant virus vector: PVX |
|---|---|---|---|
| Efficiency | High efficiency | Less efficient | Structure-function studies |
| | | | will be performed under |
| | | | the tenure of this proposal. |
| Safety | Genome integration | No risk of insertional | No risk of insertional |
| | bears risk of insertional | mutagenesis | mutagenesis |
| | mutagenesis | | |
| | Immunogenic | Less Immunogenic | Less Immunogenic |
| | Stability in biological | Aggregation and | Stability in biological |
| | media | instability in biological | media |
| | | media | |
| Manufacture | Milligrams per liter | Large-scale chemical | 1-2 grams per kilogram |
| | tissue culture | synthesis of components | leaf tissue or per liter |
| | | | culture |
| QA/C | Monodisperse | Polydisperse assemblies | Monodisperse |
| | nanoparticles with | exhibit colloidal | nanoparticles with long- |
| | long-term stability | instability | term stability |
| Materials | Exclusively spherical, | Spherical lipid-based and | New materials platform: |
| design | Enveloped/non- | polymeric formulations | filamentous, flexuous, and |
| | enveloped | | ring-shaped nucleoprotein |
| | | | assemblies with tailored |
| | | | size and mechanical |
| | | | properties |

Ring-Shaped Nanoparticle and circRNA

Applicant shows herein that PVX can be engineered and tailored for desired applications through genetic modification or bioconjugate chemistry. PVX nanofilaments measure 515×13 nm (AR 40) and consist of 1270 identical coat protein units. Essentially all 1270 coat proteins are chemically addressed via their reactive Lys side chains using NHS chemistry. Steinmetz et al, Nano Lett, 10, 305-12 (2010).

This disclosure provides a ring-shaped nanoparticle comprising a filamentous plant virus coat protein and a circularRNA (circRNA). Preferably, the coat protein is assembled on the circRNA. In some embodiments there is a plurality of plant virus coat proteins, which are the same or different from each other. In some embodiments there is a plurality of circRNAs, which are the same or different from each other. In those embodiments with more than one plant virus coat protein, the plant virus coat proteins are linked by a crosslinker to form a coat protein assembly. In this way, the plant virus coat proteins are connected to assemble around a circRNA. That is, a nanoparticle with a larger circRNA or circRNAs will have a longer coat protein assembly. In some embodiments the crosslinker is a polyethylene glycol (PEG) crosslinker, optionally a NHS- or maleimide-reactive PEG crosslinker. In some cases, the PEG crosslinker binds at a lysine and/or cysteine residue on the plant virus coat protein. In some cases the crosslinkers are branched.

In some embodiments the plant virus is PVX. In other embodiments the plant virus is Tobacco Mosaic Virus (TMV). In other embodiments the plant virus is another filamentous plant virus. In some instances the PVX coat proteins are about 100 to about 515 nm in length. In some instances the PVX coat proteins are about 250 to about 515 nm in length. In some instances there are up to about 100 crosslinkers. In some instances there are up to about 75 crosslinkers. In some instances there are up to about 50 crosslinkers. In some instances, there are up to about 25 crosslinkers. In some instances there are up to about 10 crosslinkers. In some instances there are up to about 1 crosslinker.

In some embodiments, the coat protein is a full-length coat protein of the PVX. In some instances, the coat protein comprises at least 80%, 85%, 90%, 91%, 92%, 93%, 94%, 95%, 96%, 97%, 98%, 99%, or 100% sequence identity to or consists the sequence set forth in SEQ ID NO: 1, or an equivalent thereof. In one aspect, sequence identity is determined by BLAST, run under default parameters.

(SEQ ID NO: 1)
MSAPASTTQATGSTTSTTTKTAGATPATASGLFTIPDGDFFSTAR
AIVASNAVATNEDLSKIEAIWKDMKVPTDTMAQAAWDLVRHCADV
GSSAQTEMIDTGPYSNGISRARLAAAIKEVCTLRQFCMKYAPVVW
NWMLTNNSPPANWQAQGFKPEHKFAAFDFFNGVTNPAAIMPKEGL
IRPPSEAEMNAAQTAAFVKITKARAQSNDFASLDAAVTRGRITGT
TTAEAVVTLPPP
(GenBank: ALJ33138.1)

In some embodiments, the coat protein is a full-length coat protein of the PVX. In some instances, the coat protein comprises at least 80%, 85%, 90%, 91%, 92%, 93%, 94%, 95%, 96%, 97%, 98%, 99%, or 100% sequence identity to or consists the sequence set forth in SEQ ID NO: 2, or an equivalent thereof. In one aspect, sequence identity is determined by BLAST, run under default parameters.

(SEQ ID NO: 2)
MTTPANTTQAVGSTKSTTTTTAGATPANSGLFTIPDGDFFRTAKA
VVASDAVATKEELSEIQSIWKNNKVPTDTMTQAAWTLVRHCADDG
SSAQTEMIGTGPYSNGVSRARLAAAIKEVCTLRQFCKKYAPVVWN
WMLTNNSPPANWQAQGFKPEHKFAAFDFFDGVTNPAAITPKEGLM
RPPSEAEMNAAQTAAFVKITKARAQSNDFASLDAAVTRGRITGTT
VAEAVVSLPPP
(GenBank: AAA47181.1)

In some embodiments, the coat protein is a truncated coat protein. In some instances, the truncated coat protein is a biologically functional coat protein, and is capable of assembly a ribonucleoprotein complex in the presence of a circRNA. In some cases, the truncated coat protein comprises an N-terminal deletion. In some cases, the N-terminal deletion comprises a deletion of the first 2, 3, 4, 5, 6, 7, 8, 9, 10, 11, 12, 13, 14, 15, 20, 21, 22, 23, 24, 25, or more residues from the N-terminus. In some cases, the deletion is the first 2, 3, 4, 5, 6, 7, 8, 9, 10, 11, 12, 13, 14, 15, 20, 21, 22, 23, 24, 25, or more residues from the N-terminus of SEQ ID NO: 1, or an equivalent thereof. In other cases, the deletion is the first 2, 3, 4, 5, 6, 7, 8, 9, 10, 11, 12, 13, 14, 15, 20, 21, 22, 23, 24, 25, or more residues from the N-terminus of SEQ ID NO: 2, or an equivalent thereof.

The length of the circRNA defines the size of the coat protein assembly and the ring-shaped nanoparticle. To yield efficient assembly and to obtain higher aspect ratio particles, additional non-coding sequences can be added upstream of the OAS. In some instances, the circumference of the nanoparticle is about 200 to about 2000 nanometers in circumference (see FIG. 2C for similar mRNA or gRNA-PVX nanoparticle data). In some instances the circumference of the nanoparticle is about 200 to about 515 nanometers in circumference.

In come embodiments, the circRNA are part of a circRNA cassette. In some embodiments, the circRNA cassette also includes one or more of an internal ribosome entry site (IRES) and the PVX origin of assembly site (OAS). In some embodiments, to achieve self-amplifying RNA, the circRNA cassette includes RNA-dependent RNA polymerase (RdRp).

In some embodiments, to achieve efficient translation in the target cell, regulatory elements are added to the circRNA cassette: the 5′ Cap structure, a 7-methyl-guanosine residue joined to the 5′-end via a 5′-5′ triphosphate as well as a poly(A) tail (additional regulatory elements, such as internal ribosome entry sites⁴ could also be included if deemed necessary). The polyA tail are included in the sequence and the 5′Cap are appended to the gene either post in vitro transcription using capping enzymes (e.g. New England Biolabs) or it is also possible to obtain capped mRNA by transcription through addition of the dinucleotide m7G(5′)-ppp-(5′)G. Encapsulation of the mRNA is achieved through in vitro assembly or expression in Nicotiana benthamiana.

In some aspects, the circular RNA of the nanoparticle is produced by in vitro back-splicing of a precursor RNA comprising a coding exon flanked by group II intron sequences and twister ribozymes, followed by RNase R digestion to remove linear RNA contaminants. In another aspect, the circular RNA is free of detectable linear RNA as determined by electrophoresis and resistant to RNase A digestion for at least 60 minutes at 37° C. In yet a further aspect, the nanoparticle exhibits a median diameter between about 180 and about 220 nm by transmission electron microscopy when assembled on a circular RNA of 1,800-2,400 nucleotides. In a yet further aspect, the size exclusion chromatography of the nanoparticle yields an elution volume shifted at least 0.2 mL relative to an otherwise identical nanoparticle

In one embodiment, the nanoparticle remains structurally intact after at least seven days in cell culture medium containing 10% serum without visible aggregation by dynamic light scattering. In another embodiment, the circular RNA encodes an immunomodulatory protein selected from GM-CSF, IL-2, IL-10, or a tumor antigen.

In a yet further aspect, the ring-shaped or halo-shaped nanoparticle of this disclosure further comprises a self-amplifying circular RNA replicon encoding an RNA-dependent RNA polymerase and a cytokine, wherein the replicon and cytokine sequences are separated by a self-cleaving peptide.

In another aspect, the nanoparticle exhibits a rupture force at least 1.5-fold greater than a linear RNA-loaded nanoparticle of the same coat protein, as measured by single-molecule force spectroscopy.

In certain embodiments, the circRNA comprises a nucleotide sequence encoding a polypeptide of therapeutic, diagnostic, prophylactic, or experimental utility. The encoded polypeptide can be any protein, peptide, or functional domain thereof, and may be selected from cytokines, chemokines, growth factors, tumor-associated antigens, antibody fragments, enzymes, or reporter proteins. Non-limiting examples of cytokines include granulocyte-macrophage colony-stimulating factor (GM-CSF), interleukin 2 (IL-2), interleukin 10 (IL-10), tumor necrosis factor alpha (TNFα), interferon gamma (IFN-7), interleukin 4 (IL-4), interleukin 13 (IL-13), interleukin 7 (IL-7), interleukin 15 (IL-15), and interleukin 21 (IL-21). In some embodiments, the circRNA encodes a tumor-associated antigen such as HER2, MUC1, gp100, melanoma-associated antigen (MART-1), or carcinoembryonic antigen (CEA) to elicit an immune response against a target cancer. In other embodiments, the circRNA encodes a reporter protein such as enhanced green fluorescent protein (EGFP), luciferase, mCherry, or other fluorescent or luminescent proteins suitable for monitoring expression, biodistribution, or pharmacokinetics.

In some embodiments, the one or more circRNA encodes one or more of: a cytokine, an immunomodulatory molecule, or a reporter protein, optionally wherein the cytokine is selected from GM-CSF, TNFα, IFN-γ, TGF-β, IL-2, IL-4, IL-10 and IL-13. In another embodiment, the circular RNA encodes an immunomodulatory protein selected from GM-CSF, IL-2, IL-10, or a tumor antigen.

In further embodiments, the circRNA encodes an enzyme or nuclease involved in genome editing or gene modification. Non-limiting examples include CRISPR-associated nucleases such as Cas9, Cas12a, or base editors, optionally linked to one or more guide RNAs expressed from the same or a separate construct. In other embodiments, the circRNA encodes an antibody or antibody fragment such as a single-chain variable fragment (scFv), single-domain antibody, nanobody, Fab fragment, or bispecific antibody. Such antibody sequences may be directed to tumor antigens, viral antigens, toxins, or immune checkpoint molecules, including but not limited to PD-1, PD-L1, or CTLA-4.

In some embodiments, the circRNA encodes a self-amplifying RNA replicon comprising an RNA-dependent RNA polymerase (RdRp) sequence derived from an alphavirus, nodavirus, or other viral genome, operably linked to a gene of interest. The gene of interest may be separated from the RdRp by a self-cleaving peptide sequence such as a 2A peptide, allowing coordinated production of both the replicase and the therapeutic polypeptide from a single translation product. The self-amplifying design can sustain intracellular RNA levels and prolong expression of the encoded protein.

The circRNA sequences may be codon optimized for the intended host cell to improve translation efficiency, and may incorporate untranslated regions (UTRs), internal ribosome entry sites (IRES), and stability-enhancing sequence elements to improve performance. The PVX origin of assembly site (OAS) sequence may be included within the circRNA to support specific binding and encapsidation by the plant virus coat protein. The circRNA can range from a few hundred to several thousand nucleotides in length, with the coding region optionally flanked by non-coding sequences to achieve the desired particle size or functional expression level.

In some embodiments, the circRNA does not encode a protein, but instead comprises a functional non-coding RNA such as a small interfering RNA (siRNA) precursor, a microRNA (miRNA) precursor, or a long noncoding RNA (lncRNA), designed to modulate gene expression in a target cell. The circular form can protect such RNA from exonuclease degradation and prolong its activity relative to linear counterparts.

In some embodiments of the ring-shaped nanoparticle disclosed herein the PVX coat protein further comprises a RNA-binding domain. In some embodiments, the RNA-binding domain is selected from a HK-domain, zinc-finger domain, or a peptide selected through phage display.

In certain embodiments, the nanoparticle of the present disclosure further comprises one or more additional therapeutic agents encapsulated within the internal cavity of the coat protein assembly, intercalated within the RNA cargo, or otherwise associated with the nanoparticle through chemical or physical means. As used herein, the term “additional therapeutic agent” refers broadly to any molecule, macromolecule, complex, or formulation that provides a prophylactic, therapeutic, or diagnostic benefit to a subject. Such agents may be selected from small molecules, nucleic acids, peptides, proteins, carbohydrates, lipids, or combinations thereof. The encapsulated therapeutic may act synergistically or additively with the nanoparticle's RNA payload, or may confer an independent mode of action.

Non-limiting examples of additional therapeutic agents include chemotherapeutic drugs such as anthracyclines, platinum compounds, taxanes, vinca alkaloids, antimetabolites, and topoisomerase inhibitors, as well as targeted cancer drugs including tyrosine kinase inhibitors, proteasome inhibitors, and poly(ADP-ribose) polymerase (PARP) inhibitors. In some embodiments, the therapeutic agent is selected from doxorubicin, daunorubicin, cisplatin, carboplatin, paclitaxel, docetaxel, vincristine, methotrexate, 5-fluorouracil, gemcitabine, etoposide, or irinotecan. In other embodiments, the therapeutic agent comprises an immune checkpoint inhibitor such as a PD-1, PD-L1, or CTLA-4 blocking antibody, or a small molecule antagonist of an immunosuppressive receptor.

In further embodiments, the additional therapeutic agent is a biologic macromolecule such as a monoclonal antibody, antibody fragment, cytokine, chemokine, growth factor, or enzyme. Suitable cytokines and chemokines include, but are not limited to, interleukin-2 (IL-2), interleukin-7 (IL-7), interleukin-12 (IL-12), interleukin-15 (IL-15), interferon-alpha (IFN-α), interferon-gamma (IFN-7), granulocyte-macrophage colony-stimulating factor (GM-CSF), CCL19, and CXCL10. In some embodiments, the agent is an antibody or antibody fragment targeting a tumor-associated antigen, a viral antigen, or an immune-regulatory receptor, such as trastuzumab, rituximab, cetuximab, or an anti-PD-L1 monoclonal antibody.

In still other embodiments, the additional therapeutic agent is a nucleic acid such as a messenger RNA (mRNA), circular RNA (circRNA), self-amplifying RNA, antisense oligonucleotide, small interfering RNA (siRNA), short hairpin RNA (shRNA), microRNA mimic or inhibitor, or a CRISPR-associated nucleic acid editing system. Such nucleic acids may be stabilized or chemically modified to enhance delivery and function when co-encapsulated with the nanoparticle. Gene editing systems may include combinations of guide RNA and nuclease components for genome modification in a target cell.

In certain embodiments, the additional therapeutic agent is an antimicrobial or antiviral compound, including antibiotics, antifungals, antiprotozoals, or antiviral nucleoside and nucleotide analogs, suitable for use against bacterial, fungal, parasitic, or viral pathogens. In other embodiments, the additional agent is a radiotherapeutic isotope, photosensitizer, or imaging contrast agent, thereby enabling theranostic applications in which the nanoparticle serves both therapeutic and diagnostic purposes.

These therapeutic agents may be encapsulated during the self-assembly process of the filamentous plant virus coat proteins around the primary RNA cargo, by passive diffusion into pre-formed particles under conditions that preserve coat protein integrity, or through covalent or non-covalent conjugation strategies targeting solvent-accessible residues or engineered reactive sites on the coat protein subunits. Encapsulation or association may be optimized to preserve the structural stability of the nanoparticle and to maintain the bioactivity of both the RNA cargo and the additional therapeutic agent.

In some embodiments, the ring-shaped nanoparticle disclosed herein further comprises, or alternatively consists essentially of, or yet further consists of a detectable marker or a purification marker. As used herein, the term detectable marker refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection are accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I. As used herein, the term purification marker or reporter protein refer to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

### Plasmids Encoding the Ring-Shaped Nanoparticle

Also provided herein are polynucleotides and methods of controlling the size of virus-like particles assembled from RNA derived from a plant virus genome, comprising generating RNA transcripts from a polynucleotide or cloning vector that comprises a promoter for in vitro transcription, an origin of assembly site sequence from said plant virus genome, and a polyadenylation signal, wherein the RNA transcript is capped and polyadenylated, and wherein the length of the plant virus genome sequence determines the particle size. A nonlimiting example of such is provided in Table 4.

In certain embodiments, the disclosure provides DNA cloning vectors configured to generate in vitro transcribed RNA molecules for the assembly of virus-like particles (VLPs) with controlled size profiles. The vectors are constructed as plasmids and include, in operable arrangement, a RNA polymerase such as a bacteriophage T7 RNA polymerase promoter positioned upstream of a polynucleotide sequence derived from a plant virus genome. Non-limiting examples of such plant viral genomes are provided herein, and in one embodiment, the plant virus is potato virus X (PVX). The polynucleotide incorporates an origin of assembly site (OAS) capable of initiating capsid assembly in the presence of the coat protein. The OAS can comprise a cis-acting sequence located in the 5′ region of the coat protein genomic RNA. In one aspect, this cis-acting element includes an AC-rich single-stranded region extending from nucleotide positions 1 through 49 of the native PVX genome, immediately followed by a downstream stem-loop structure (SL1) from nucleotide positions 49 through 85. The AC-rich segment and the SL1 structure together form the OAS, which serves as the nucleation center for virion or VLP assembly.

Downstream of the PVX genome fragment, the plasmid further contains a polyadenylation signal such as a bovine growth hormone (BGH) polyadenylation signal to enable in vitro production of transcripts with a defined polyadenylate tail. For size control, a family of plasmids can be prepared in which the coat protein sequence is progressively truncated from the 3′ end to yield constructs encoding RNA segments of varying predetermined length. In representative embodiments, the inserts encode: a full-length coat protein genome of approximately 6.4 kilobases, and truncated segments of about 5 kilobases, 4 kilobases, 3 kilobases, 2 kilobases, 1.5 kilobases, 1 kilobase, 500 nucleotides, 250 nucleotides, or 100 nucleotides. These length-selected genome fragments are designed such that they all retain the 5′ OAS region while lacking differing amounts of internal and/or 3′ sequence, thereby controlling the final dimensions of particles formed during assembly.

To prepare in vitro transcribed RNA for assembly, each plasmid construct is linearized downstream of the polyadenylation signal. In one embodiment, transcription is carried out using a T7 RNA polymerase transcription system to produce uncapped RNA molecules. The resulting transcripts are enzymatically capped with a 7-methylguanylate cap at the 5′ end (Cap-0 structure) to mimic the natural modification found on the coat protein genomic RNA. A poly(A) tail is then added enzymatically to the 3′ end, using the signal encoded in the plasmid. This capping and polyadenylation improves stability and assembly efficiency.

Following transcription, the RNA products are purified to remove enzymes, free nucleotides, and DNA template. In one embodiment, purification is accomplished by lithium chloride precipitation, which selectively precipitates RNA and leaves residual proteins and DNA in solution. Purified RNAs are then analyzed on agarose gels under non-denaturing conditions, for example, using 1.2% weight/volume agarose in tris-borate-EDTA buffer, to verify the expected length and integrity. To confirm that the plasmid templates are correct before transcription, the PVX insert is sequenced by either Sanger sequencing or nanopore sequencing, depending on desired throughput and read length requirements.

Assembly of size-controlled VLPs is initiated by combining the purified RNA transcripts with coat protein in vitro under assembly-permissive conditions. Because the OAS is present in all constructs, capsid assembly starts from the 5′ end and proceeds along the RNA. By shortening the RNA length, the assembly process terminates earlier, producing particles of reduced overall dimensions, while longer transcripts yield particles approaching the size of native PVX virions. The relationship between RNA genome length and particle size is reproducible and predictable across the full range of constructs, enabling fine control over VLP size distribution for research, diagnostic, and therapeutic applications.

The disclosure encompasses both the specific embodiments described above and, more broadly, the principle of controlling the size of plant-virus-derived VLPs through manipulation of the genome length in in vitro transcription systems. Substitution of the PVX genome with sequences from other plant viruses having a defined origin of assembly site, and variation in promoter, capping, or polyadenylation methods, can be used without departing from the disclosure's scope, provided that the transcript length is selected to yield a desired particle size. This flexibility extends protection to any implementation where a designer RNA genome fragment containing a functional OAS is transcribed, capped, tailed, and assembled into VLPs whose dimensions are determined by the length of the RNA template.

### Compositions Comprising the Ring-Shaped Nanoparticle

In one aspect, provided herein is a composition comprising, or alternatively consisting essentially of, or yet further consisting of a carrier and one or more of the ring-shaped nanoparticle of this disclosure. The ring-shaped nanoparticle of the present disclosure can be bound to many different carriers. Thus, this disclosure also provides compositions containing the ring-shaped nanoparticle and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the disclosure. Those skilled in the art will know of other suitable carriers for binding viral nanoparticle filaments, or will be able to ascertain such, using routine experimentation.

In another aspect, the composition further comprises an additional therapeutic agent. In some embodiments, the additional therapeutic agent can be encapsulated within a cavity of the ring-shaped nanoparticle. In some embodiments the additional therapeutic agent is an anti-cancer therapy.

In some embodiments, the additional therapeutic agent comprises chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

In some cases, the additional therapeutic agent comprises an inhibitor of the enzyme poly ADP ribose polymerase (PARP). Exemplary PARP inhibitors include, but are not limited to, olaparib (AZD-2281, Lynparza®, from Astra Zeneca), rucaparib (PF-01367338, Rubraca®, from Clovis Oncology), niraparib (MK-4827, Zejula®, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some cases, the additional therapeutic agent comprises an immune checkpoint inhibitor. Exemplary checkpoint inhibitors include:
PD-L1 inhibitors such as Genentech's MPDL3280A (RG7446), anti-PD-L1 monoclonal antibody MDX-1105 (BMS-936559) and BMS-935559 from Bristol-Meyer's Squibb, MSB0010718C, and AstraZeneca's MEDI4736;
PD-L2 inhibitors such as GlaxoSmithKline's AMP-224 (Amplimmune), and rHIgM12B7;
PDF-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (Opdivo®, BMS-936558, MDX1106), AstraZeneca's AMP-514 and AMP-224, and Pidilizumab (CT-011) from CureTech Ltd;
CTLA-4 inhibitors such as Bristol Meyers Squibb's anti-CTLA-4 antibody ipilimumab (also known as Yervoy®, MDX-010, BMS-734016 and MDX-101), anti-CTLA4 antibody clone 9H10 from Millipore, Pfizer's tremelimumab (CP-675,206, ticilimumab), and anti-CTLA4 antibody clone BNI3 from Abeam;
LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12;
B7-H3 inhibitors such as MGA271;
KIR inhibitors such as Lirilumab (IPH2101);
CD137 inhibitors such as urelumab (BMS-663513, Bristol-Myers Squibb), PF-05082566 (anti-4-1BB, PF-2566, Pfizer), or XmAb-5592 (Xencor);
PS inhibitors such as Bavituximab;
and inhibitors such as an antibody or fragments (e.g., a monoclonal antibody, a human, humanized, or chimeric antibody) thereof, RNAi molecules, or small molecules to TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM.

In some cases, the additional therapeutic agent comprises pembrolizumab, nivolumab, tremelimumab, or ipilimumab.

In some cases, the additional therapeutic agent comprises an antibody such as alemtuzumab, trastuzumab, ibritumomab tiuxetan, brentuximab vedotin, ado-trastuzumab emtansine, or blinatumomab.

In some cases, the additional therapeutic agent comprises a cytokine. Exemplary cytokines include, but are not limited to, IL-Iβ, IL-6, IL-7, IL-10, IL-12, IL-15, IL-21, or TNFα.

In some embodiments, the additional therapeutic agent comprises a receptor agonist. In some instances, the receptor agonist comprises a Toll-like receptor (TLR) ligand. In some cases, the TLR ligand comprises TLR1, TLR2, TLR3, TLR4, TLR5, TLR6, TLR7, TLR8, or TLR9. In some cases, the TLR ligand comprises a synthetic ligand such as, for example, Pam3Cys, CFA, MALP2, Pam2Cys, FSL-1, Hib-OMPC, Poly I:C, poly A:U, AGP, MPL A, RC-529, MDF2p, CFA, or Flagellin.

In some cases, the additional therapeutic agent comprises an adoptive T cell transfer (ACT) therapy. In one embodiment, ACT involves identification of autologous T lymphocytes in a subject with, e.g., anti-tumor activity, expansion of the autologous T lymphocytes in vitro, and subsequent reinfusion of the expanded T lymphocytes into the subject. In another embodiment, ACT comprises use of allogeneic T lymphocytes with, e.g., anti-tumor activity, expansion of the T lymphocytes in vitro, and subsequent infusion of the expanded allogeneic T lymphocytes into a subject in need thereof.

Pharmaceutical compositions of the present disclosure can be administered in a manner appropriate to the disease to be treated or prevented. The quantity and frequency of administration will be determined by such factors as the condition of the patient, and the type and severity of the patient's disease, although appropriate dosages can be determined by clinical trials.

In some embodiments, the pharmaceutical composition and formulations described herein are administered to a subject by multiple administration routes, including but not limited to, parenteral, oral, buccal, rectal, sublingual, or transdermal administration routes. In some cases, parenteral administration comprises intravenous, subcutaneous, intramuscular, intracerebral, intranasal, intra-arterial, intra-articular, intradermal, intravitreal, intraosseous infusion, intraperitoneal, or intratechal administration. In some instances, the pharmaceutical composition is formulated for local administration. In other instances, the pharmaceutical composition is formulated for systemic administration.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pennsylvania 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins1999).

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®, dibasic calcium phosphate, dicalcium phosphate dihydrate, tricalcium phosphate, calcium phosphate, anhydrous lactose, spray-dried lactose, pregelatinized starch, compressible sugar, such as Di-Pac® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel®PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol©), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™ sodium oleate, sodium benzoate, glyceryl behenate, polyethylene glycol, magnesium or sodium lauryl sulfate, colloidal silica such as Syloid™, Cab-O-Sil©, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, poly sorbate-20 or Tween® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

In some embodiments, the pharmaceutical compositions described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously, alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

Methods of Treatment with the Ring-Shaped Nanoparticle or Composition

The ring-shaped nanoparticle and/or the composition of the present disclosure can be used to deliver a circRNA to a cell. In some embodiments, the circRNA is delivered by contacting the cell with the ring-shaped nanoparticle in vivo. In other embodiments, the circRNA is delivered by contacting the cell with the ring-shaped nanoparticle in vitro. Contacting can be in vitro or in vivo.

The ring-shaped nanoparticle and/or the composition of the present disclosure can be used to treat tumors and cancer. The ring-shaped nanoparticle and/or the composition provided herein can be administered either alone or in combination with diluents, known anti-cancer therapeutics, and/or with other components such as cytokines or other cell populations that are immunostimulatory. They can be administered as a first line therapy, a second line therapy, a third line therapy, or further therapy. The disclosed ring-shaped nanoparticle and/or composition can be combined with other therapies (e.g., chemotherapy, radiation, surgery etc.). Non-limiting examples of additional therapies include chemotherapeutics or biologics. Appropriate treatment regimens will be determined by the treating physician or veterinarian. In one embodiment, disclosed herein is a method of inhibiting the growth of a tumor and/or treating a cancer and/or preventing relapse of cancer in a subject in need thereof, comprising, or alternatively consisting essentially of, or yet further consisting of administering to the subject an effective amount of the ring-shaped nanoparticle and/or the composition provided herein.

In one embodiment, the tumor is a solid tumor. The solid tumor could be a melanoma, a colon carcinoma, a breast carcinoma and/or a brain tumor. In one aspect, the cancer to be treated is a carcinoma, sarcoma, or blood cancer.

The methods are useful to treat subjects such as humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). A mammal can be any age or at any stage of development (e.g., an adult, teen, child, infant, or a mammal in utero). A mammal can be male or female. In certain embodiments the subject has or is suspected of having a neoplastic disorder, neoplasia, tumor, malignancy or cancer.

For the above methods, an effective amount is administered, and administration of the cell or population serves to attenuate any symptom or prevent additional symptoms from arising. When administration is for the purposes of preventing or reducing the likelihood of cancer recurrence or metastasis, the cell or compositions can be administered in advance of any visible or detectable symptom. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, epidural and intrathecal.

The methods provide one or more of: (1) preventing the symptoms or disease from occurring in a subject that is predisposed or does not yet display symptoms of the disease; (2) inhibiting the disease or arresting its development; or (3) ameliorating or causing regression or relapse of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies e.g., surgical recession, chemotherapy, radiation. In one aspect, treatment excludes prophylaxis.

### Production Methods

Also provided are methods of making a ring-shaped or halo-shaped nanoparticle comprising: (a) in vitro transcribing a precursor RNA comprising a coding exon flanked by group II intron sequences and twister ribozymes; (b) treating the precursor RNA with heat-cool cycles to promote back-splicing and circularization; (c) digesting the RNA with RNase R to remove linear RNA species; and (d) contacting the circular RNA with purified filamentous plant virus coat protein under assembly conditions to form the nanoparticle. In one aspect, the filamentous plant virus coat protein is purified from Potato Virus X propagated in Nicotiana benthamiana plants, disassembled into coat protein subunits, and reassembled onto the circular RNA at a coat protein-to-RNA mass ratio of 40:1.

In a further aspect, the filamentous plant virus coat protein is recombinantly expressed in Escherichia coli with an N-terminal histidine tag, purified, and mixed with native coat protein prior to the contacting step to yield nanoparticles comprising both recombinant and native coat proteins. In another aspect, the contacting step is performed in an assembly buffer comprising about 10 mM Tris-HCl, about pH 8.0, and about 50 mM NaCl, and includes sequential addition.

In a further embodiment, the methods further comprise crosslinking adjacent coat protein subunits in the assembled nanoparticle with a bifunctional polyethylene glycol (PEG) crosslinker targeting lysine or cysteine residues, to increase rupture force by at least 1.5-fold compared to an uncrosslinked nanoparticle. In another aspect, the circular RNA encodes a self-amplifying replicon comprising an RNA-dependent RNA polymerase and a cytokine sequence separated by a self-cleaving peptide, and wherein the nanoparticle is assembled to contain said replicon circular RNA payload.

### Examples

The examples demonstrate PVX as an exemplary filamentous nanoparticle for mRNA delivery and describes an additional design of filamentous mRNA-laden PVX and discloses the properties of PVX that is assembled on a circRNA to form a ring-shaped nanoparticle. Additional examples focus on therapeutic targets such as expression of cytokine granulocyte macrophage colony-stimulating factor (GM-CSF) as well as interleukin2 (IL2). Expression of GM CSF or IL2 holds great potential in cancer immunotherapy. For example, expression of GM-CSF within the tumor can overcome the immune-suppressive state therefore reprogramming the tumor microenvironment, leading to the recruitment of immune cells to prime local and systemic anti-tumor immune responses. GM-CSF is encoded by IMLYGIC®, an oncolytic virus therapy that was recently FDA-approved for the treatment of melanoma patients. Similarly, IL2 is a cytokine produced by activated T cells to support T cell growth and proliferation. IL2 therapy has shown promise by improving patient survival when given in combination with cancer vaccines. However, while IL2 therapy is FDA-approved to treat metastatic melanoma, high systemic doses of IL2 are needed, leading to severe toxicity. Data suggest efficacy of local IL2 expression, e.g. intratumoral expression of IL2, improves antitumor effects of CAR-T cells.

Results from the examples provide novel insights into the bioengineering design principles to deploy plant virus-based vectors in nucleic acid therapy and to provide a framework for understanding how size, mechanical properties, and geometry can function as handles to optimize performance of the system.

The disclosed methodology can be adapted as needed. The same methodology described in the examples can be adapted and applied to other RNAs and nucleotides and can be applied to other plant viruses.

### Example 1

### Materials and Methods

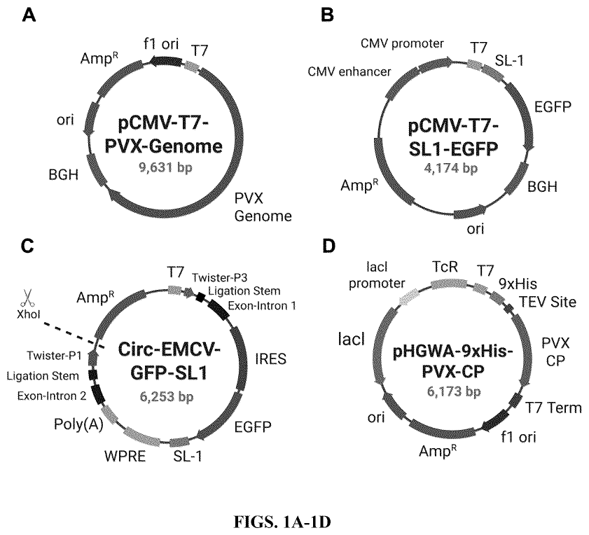

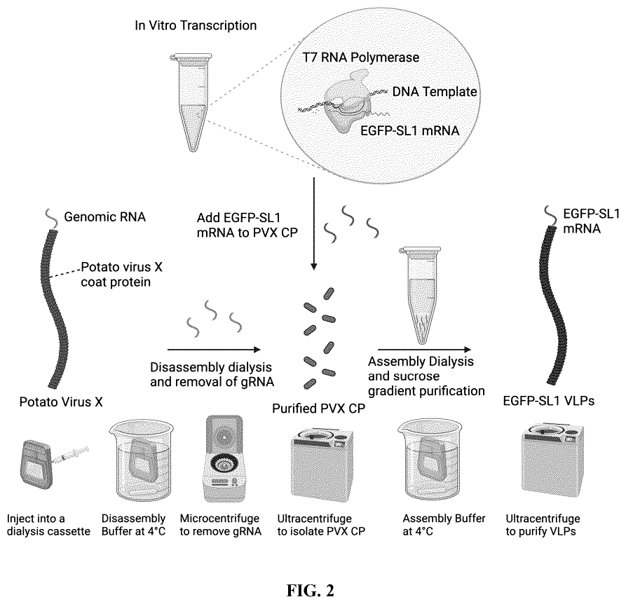

Molecular Cloning. The plasmids used in this study included pCMV-T7-EGFP (BPK1098), a gift from Benjamin Kleinstiver and Harvard University (Addgene plasmid #133962; http://n2t.net/addgene:133962; RRID: Addgene_133962) and Circ-oc-EMCV-GFP, a gift from Prashant Mali and UC San Diego (Addgene plasmid #226261; http://n2t.net/addgene:226261; RRID:Addgene_226261). PVX genome plasmids for size control and mixed assembly studies were adapted from pCMV-T7 to include the full PVX genome (pCMV-T7-PVX-Genome; FIG. 1A). To promote PVX coat protein (CP) binding and in vitro assembly of PVX VLPs on in vitro transcripts, the origin of assembly site (SL1, 60 nts)⁶ was cloned downstream of the T7 promoter and upstream of the EGFP fluorescent reporter gene using gBlocks (Integrated DNA Technologies, San Diego, CA) and Gibson assembly (Gibson Assembly Master Mix, New England Biolabs, Ipswich, MA) to yield pCMV-T7-SL1-EGFP (FIG. 1B). For circRNA, SL1 was cloned downstream of EGFP and before the WPRE sequence to generate Circ-EMCV-GFP-SL1 (FIG. 1C). Another plasmid, pHGWA-9×His-PVX-CP, was cloned to contain the PVX CP downstream of an N-terminal 9×His tag and TEV cleavage site (FIG. 1D) for mixed assembly VLPs. A cell line derivative of DH5a (NEB 5-alpha Competent E. coli, New England Biolabs, Ipswich, MA) was used for cloning the vector constructs and BL21(DE3) (NEB T7 Competent E. coli, New England Biolabs, Ipswich, MA) for protein production; sequences were confirmed through either Sanger sequencing (Eurofins Genomics, Louisville, KY) or Nanopore sequencing (Plasmidsaurus, South San Francisco, CA) of plasmid DNA. The plasmid sequences are provided by Addgene as well as the Supporting Information and the gBlocks are detailed in the Supporting Information (Tables 3 and 4).

In vitro Transcription (IVT). DNA templates for RNA products were created through PCR amplification (Q5® High-Fidelity 2× Master Mix, New England Biolabs, Ipswich, MA) from plasmid PCR products then amplified using the T7 promoter and BGH poly(A) sites for the forward and reverse primers (Integrated DNA Technologies, San Diego, CA) respectively. Plasmids and designs for circRNA included additional features such as 5′ ribozyme sequence, a 5′ ligation sequence, an IRES sequence linked to the product of interest, a 3′ UTR sequence, a 3′ ligation sequence, a 3′ ribozyme sequence, and a poly-T stretch to terminate transcription. PCR reaction mixtures were treated with DpnI (1 U/μl, New England Biolabs, Ipswich, MA) to degrade template pDNA and PCR products were subsequently purified using a QIAquick PCR Purification Kit (Qiagen, Valencia, CA). The PCR products were then used to generate pre-mRNA using T7 High Yield RNA Synthesis Kits (New England Biolabs, Ipswich, MA) per the manufacturer's specifications. In vitro transcription (IVT) was carried out in a heat block for 1-2 hours at 37° C. IVT pre-mRNA products were then purified using LiCl precipitation. To complete the processing of pre-mRNA into mature linear mRNAs, a 7-methylguanylate cap (Cap-0) structure was added to the 5′ end of the mRNA using the Vaccinia Virus Capping System (New England Biolabs, Ipswich, MA) and then purified using LiCl before poly(A) tailing using E. coli Poly(A) Polymerase (New England Biolabs, Ipswich, MA) according to the vendor's protocols. The final mRNA was then purified using LiCl precipitation once more, followed by quantification by UV/Vis Spectroscopy (Nanodrop 2000, Thermo Fisher, USA) to measure purity (RNA with a A260/A280 ratio between 2.0-2.2 was for experimentation exclusively). Characterization and imaging of mRNA and circRNA sizes were confirmed by gel electrophoresis using a 1.2% (w/v) agarose gel in electrophoresis buffer (1× Tris-Borate-EDTA (TBE) buffer, pH 8.3) and Agilent 2100 bioanalyzer (Agilent, Santa Clara, CA) RNA electropherograms. mRNA was stored at either −20° C. or −80° C. after IVT and between uses.

In regard to the IVT protocol for circRNA, plasmids containing the RNA template were linearized at the 3′ end of the template sequence using XhoI (New England Biolabs, Ipswich, MA) digestions; the linearized product was then purified using the QiaQuick PCR spin columns. RNA products were then produced using these linearized plasmid templates using the HiScribe T7 Quick High Yield RNA Synthesis Kit (NEB E2040) per the manufacturer's protocol. IVT RNA reactions were cleaned with the Monarch RNA Cleanup Kit (500 μg) (T2050) according to manufacturer's instructions. CIP treatment was then performed to ensure the removal of any remaining triphosphates from IVT, including cleaved twister ribozyme product. Cellulose chromatography was performed twice according to Baiersdorfer, M. et al per 100 μg of RNA.²⁹ To isolate circular RNA products, 20 μg of circRNA was diluted to a total volume of 88 μL then heated at 65° C. for 5 min and subsequently cooled on ice before adding 30U RNase R (2 μL) and 10 μL of 10× RNase R buffer (MCLAB) and the reaction incubated at 37° C. for 30 min.

Preparation of PVX CPs and RNA. PVX virions were propagated and purified from infected Nicotiana benthamiana plants as previously described.¹⁸ To isolate CP, a 5 mg suspension of purified PVX in potassium phosphate buffer (0.1 M KP, pH 7.0) was aliquoted into a 10 kDa MWCO Slide-A-Lyzer Dialysis Cassette (Thermo Fisher Scientific, Waltham, MA) and dialyzed against a ‘disassembly buffer’: 50 mM Tris-Cl, pH 7, 750 mM CaCl₂, 1 mM EDTA, 1 mM DTT, and 0.5 mM PMSF for 8-24 hours at 4° C. to disassemble the virions. Dissociated suspensions were then centrifuged at 20,000 g for 20 min at 4° C. to remove gRNA, followed by ultracentrifugation at 180,000 g for 1 hour and 10 min at 4° C. to remove any intact virions. gRNA pellets were resuspended in nuclease-free water and precipitated using LiCl precipitation. PVX CP suspensions were then dialyzed against an ‘assembly buffer’ containing 10 mM Tris-HCl, pH 8.0, and 50 mM NaCl for 8-24 hours at 4° C. This was followed by centrifugation at 20,000 g for 20 min at 4° C. to remove any residual gRNA, followed by ultracentrifugation at 180,000 g for 1 hour and 10 min at 4° C. to remove any reassembled VLPs. The CP was concentrated using Amicon 10 kDa MWCO Ultra Centrifugal Filter tubes (Millipore Sigma, Burlington, MA) and the concentration was determined through a Pierce BCA Protein assay (Thermo Fisher Scientific, Waltham, MA). The integrity of the CP and the removal of residual gRNA were confirmed by size-exclusion chromatography, chromatography AKTA Explorer chromatography system with a Superose 6 Increase column (GE Healthcare, Chicago, IL); the A260:280 nm ratio of pure CP is <0.57.

In vitro assembly of PVX VLPs with mRNA, circRNA, or PVX gRNA. Assembly reactions with in vitro transcribed EGFP-SL1 mRNAs, genomic fragment mRNAs, Circ-EGFP-SL1, and PVX gRNA were carried out at room temperature using a 50:1 CP:RNA mass ratio using assembly buffer for 3-24 hours at concentrations between 1-5 mg/mL (FIG. 2). Assembled VLPs were then centrifuged at 20,000 g for 20 min at 4° C. to remove any residual RNA. Each in vitro assembled VLP preparation was purified through a sucrose cushion gradient (30% in Tris-buffered saline [TBS]—150 mM NaCl, 50 mM Tris-HCl, pH 7.6) and ultracentrifuged at 180,000 g for 1 hour and 10 min at 4° C. VLP pellets were resuspended in either assembly or potassium phosphate buffer (0.01 M KP, pH 7.0) with 5-10% sucrose (w/v).

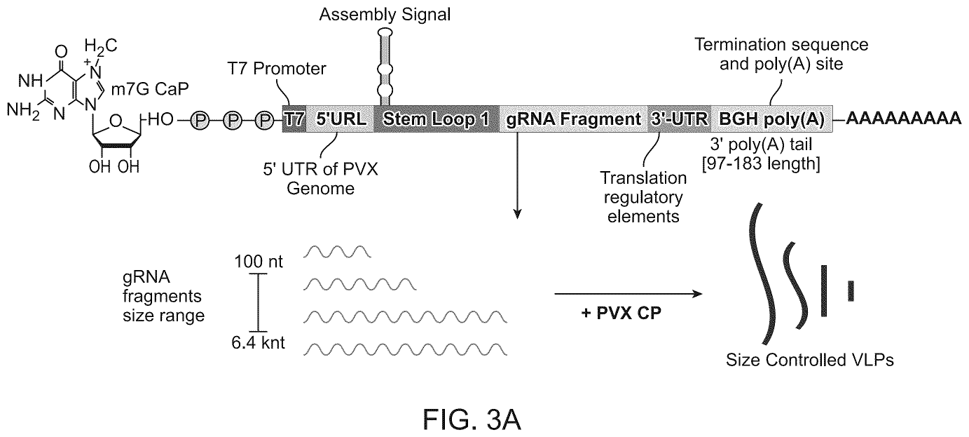

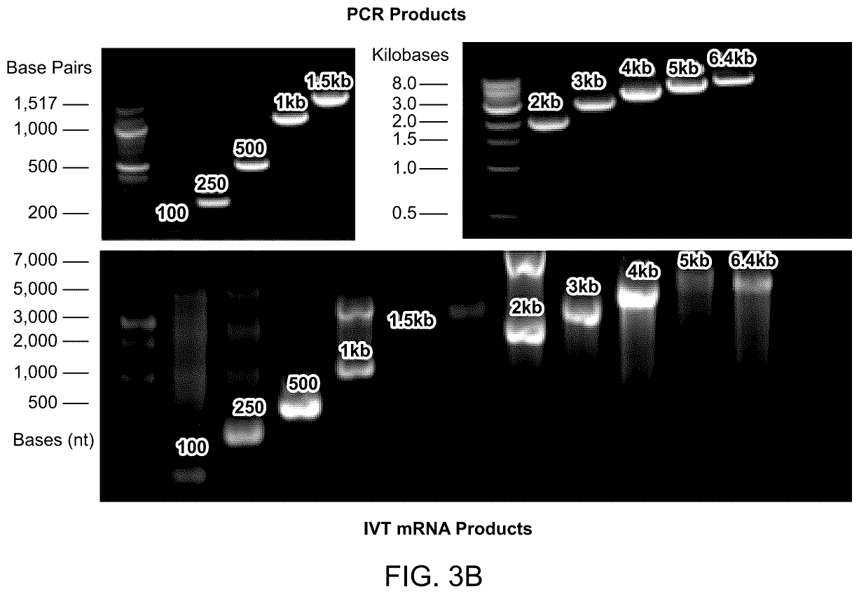

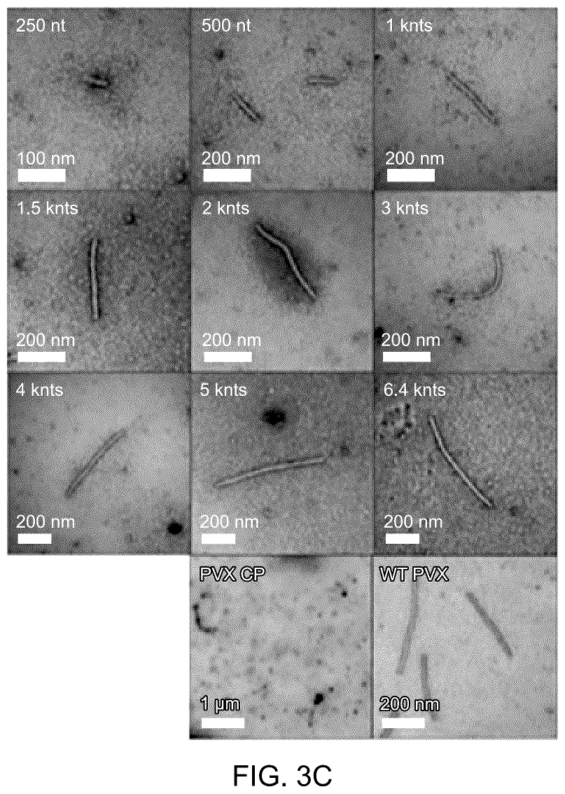

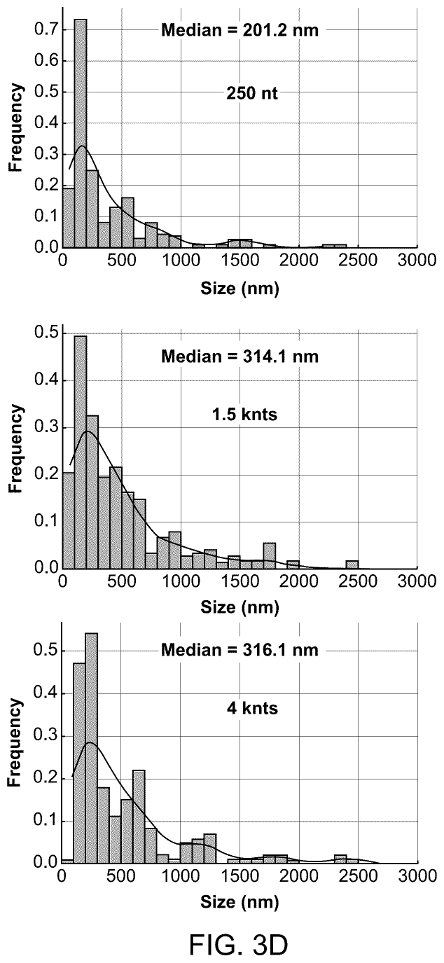

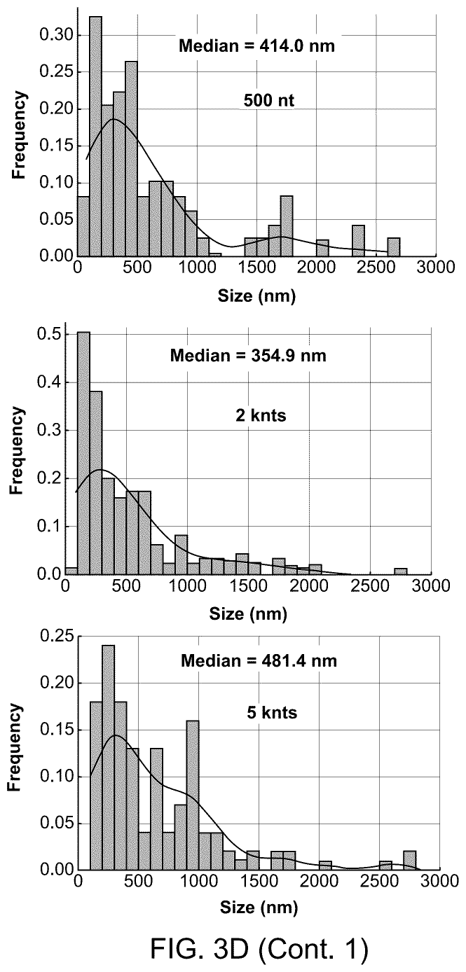

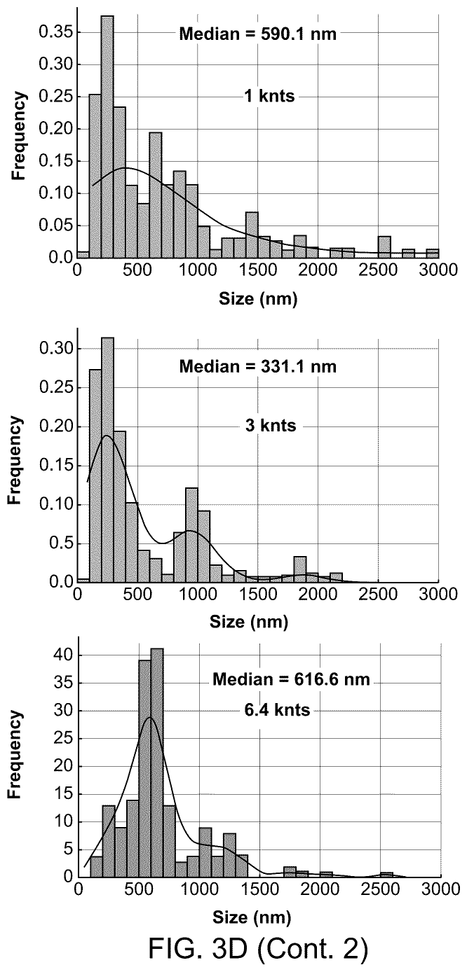

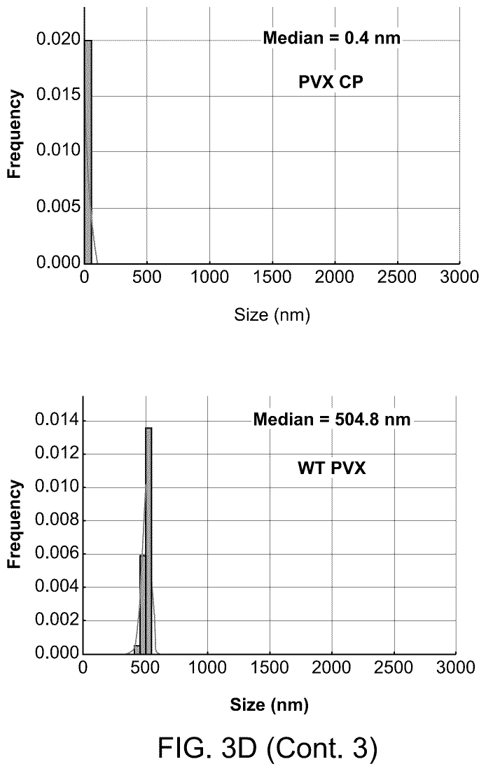

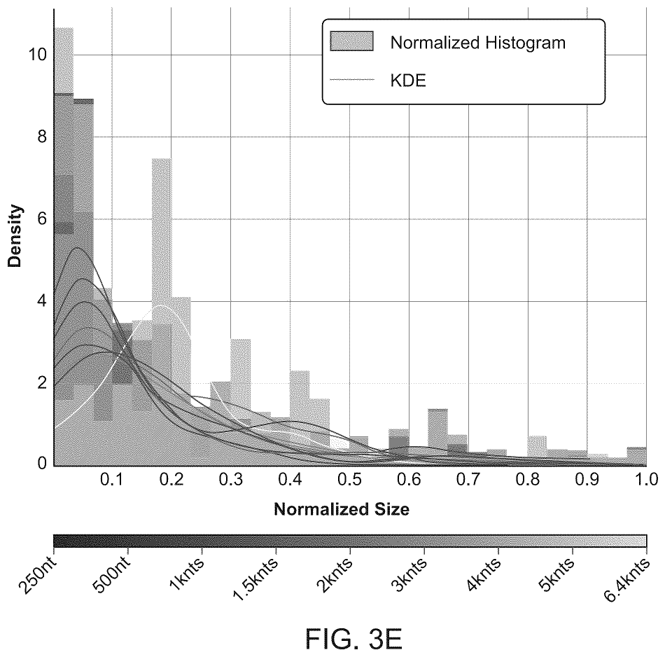

Cloning and in vitro assembly of Size-Controlled VLPs. In vitro transcribed size-controlled mRNA transcripts, derived from gRNA sequences cloned into the pCMV-T7-PVX-Genome plasmid, were designed as shown in FIG. 3A. The constructs include a T7 promoter, the PVX genome, and a BGH poly(A) site. The PVX origin of assembly site (OAS) sequence consists of a cis-acting element from the 5′ region of PVX gRNA that includes an AC-rich single-stranded region from nt 1-49 and downstream SL1 located at nt 49-85, which forms the OAS. To produce PVX VLPs of distinct sizes, vectors were designed with the full-length PVX RNA genome of 6.4 kb and smaller genomic fragments (shortened at the 3′ end) of 5 kb, 4 kb, 3 kb, 2 kb, 1.5 kb, 1 kb, 500 bases, 250 bases and 100 bases (FIG. 3A). These were then in vitro transcribed using a T7 RNA polymerase transcription kit, capped with a 7-methylguanylate cap (m7G; Cap-0), and poly(A) tailed. Following IVT, products were LiCl purified and characterized using 1.2% (w/v) agarose gels in TBE (FIG. 3B), and template plasmids were confirmed through either Sanger or Nanopore sequencing.

Characterization of Assembled VLPs. VLP and PVX morphology and structure were examined with a JEOL 1400 plus transmission electron microscope (TEM) (CMM Electron Microscopy Facility, San Diego, CA) at 80 kV with a Gatan OneView 4 k×4 k camera. VLPs and PVX (each at 0.1 mg/ml) were prepared for TEM using glow-discharged carbon paper-coated copper (200-mesh) PELCO Pinpointer grids (Ted Pella, USA). After a 2 min sample deposition step (repeated twice), the grids were blotted on Whatman filter paper and washed twice with nuclease-free water (1 min each) before negative staining with 2% (w/v) uranyl acetate (10 μl) for 30 seconds. Additional steps were performed for gold nanoparticle-conjugated VLPs as described below. Images were captured at 15 kX magnification and processed using Fiji biological-image analysis software (v2.9) to determine VLP length and diameter through pixel-to-nanometer conversions. Purity and size distribution profiles of VLPs were determined through their elution profiles on an AKTA Explorer chromatography system with a Superose 6 Increase column (GE Healthcare, Chicago, IL, USA). VLPs were run in assembly buffer (10 mM Tris-HCl, pH 8.0, and 50 mM NaCl) at a concentration of 0.1 mg/mL at full column volume at a flow rate of 0.5 mL/min.

Characterization of mixed VLP assemblies by immunogold TEM and western blot. For mixed VLP assemblies containing a ratio of recombinant (His-tagged):wildtype CPs, 10 nm Ni-NTA-Nanogold® (Nanoprobes, Yaphank, NY), diluted 1/30 in nuclease-free water, was added to the grid after sample deposition (see above) followed by a 30-minute incubation at room temperature and a 1 minute wash with 20 mM Tris at pH 7.6 with 150 NaCl containing 8 mM imidazole. Additionally, mixed VLP assemblies were characterized using Goldiblot™ His-tag Western Blot Kits (Nanoprobes, Yaphank, NY). CPs were separated by 4-12% SDS-PAGE and then transferred to PVDF or nitrocellulose membranes. Membranes were then equilibrated with 20 mM Tris, 0.15 M NaCl, pH 7.6, 0.1% (w/v) Tween®-20 (TBS-0.1% T) for 3 min, blocked with 5% (w/v) nonfat dry milk in TBS-0.1% T for 15 min, and then incubated with 0.1 ml of Nickel-NTA-Nanogold® added to 10 mL of 1% (w/v) nonfat dry milk in 20 mM Tris, 0.15 M NaCl pH 7.6, 0.6% (w/v) Tween®-20 (TBS-0.6% T) for 30 min, followed by washing the membrane twice with 15 mL of 10 mM imidazole in TBS-0.6% T for 2 min each, washing the membrane three times with deionized water for 3 min each, simultaneously mixing the Goldiblot™ AutoMet Detect ABCD mix, adding ABCD mix to sample for 6-20 min, and washing three times with deionized water for 3 min each time before drying. After fully dried, membranes were imaged using a FluoroChem R System (ProteinSimple, San Jose, CA, USA) using the epi-white illumination setting.

Transfection of mRNA, circRNA, and VLPs. Free EGFP-SL1 mRNA, RNase A-treated mRNA, mRNA-containing VLPs, and RNase A-treated VLPs were used to transfect BHK-21 cells (ATCC, Manassas, VA). 12-well plates were seeded with 100,000 cells/well and 1 ml Dulbecco's Modified Eagle Medium (DMEM, Thermo Fisher Scientific, Waltham, MA) supplemented with 10% (v/v) fetal bovine serum (FBS, Thermo Fisher Scientific, Waltham, MA) and 1% (w/v) penicillin/streptomycin (Thermo Fisher Scientific, Waltham, MA) per well the day before transfection to reach 60-80% confluency. BHK-21 cells were then transfected using 1-2 μg/well of mRNA or 100-300 μg/well VLPs and 5-12.5 μl of Lipofectamine 2000 (Thermo Fisher Scientific, Waltham, MA) according to the manufacturer's guidelines and specifications. circRNA controls and VLPs were treated similarly for HEK-293T cells plated onto 12-well plates at 100,000 cells/well. VLPs were first diluted into a 20 mM HEPES solution for 10 min before forming complexes with Lipofectamine in Opti-MEM (Thermo Fisher Scientific, Waltham, MA). For RNase-treated conditions, RNase A (PureLink RNase A; Thermo Fisher Scientific, Waltham, MA) digestion was performed at a ratio of 2 ng RNase A to 1 μg VLPs and 0.5 ng RNase A to 1 μg RNA for 1 hr at 4° C. before encapsulation. RNase digestion was terminated through the addition of a 1:1 ratio of Ribolock RNase Inhibitor (Thermo Fisher Scientific, Waltham, MA).

Quantitative RT-PCR of Disassembled VLP mRNAs. To confirm the ability of VLPs to encapsulate and protect EGFP-SL1 mRNA, VLP and control suspensions were dialyzed overnight in disassembly buffer followed by centrifugation at 20,000 g at 4° C. for 20 min to pellet the mRNA. The pellets were then resuspended in nuclease-free water and precipitated using LiCl. Following mRNA purification, cDNA synthesis was performed using an RT2 First Strand Kit (Qiagen, Valencia, CA). The quantitative RT-PCR assay was then executed using RT2 SYBR® Green qPCR Mastermixes (Qiagen, Valencia, CA) along with transgene specific primers (T7 forward 5′-TAATACGACTCACTATAGGGAGAGC-3′, BGH 5′-TAGAAGGCACGTCGAGGC-3′). EGFP-SL1 mRNA was used as a positive control in addition to PVX virions, gRNA VLPs, and CP as negative controls. EGFP mRNA was used as the relative expression internal control gene and real-time efficiencies (E) were calculated using the crossing point difference (CPA). The RT-PCR assay was executed on a Biorad CFX96 Real-Time System thermal cycler (Biorad, Hercules, CA). Each assay sample set was performed in triplicate.

Flow cytometry. Transfected BHK-21 and HEK 293T cell lines were harvested using Gibco Trypsin-EDTA (0.05% [w/v]) (Thermo Fisher Scientific, Waltham, MA), followed by centrifugation at 500 g, washing with 1×PBS, pH 7.4, fixation with 4% (w/v) paraformaldehyde in PBS for 10 min, and resuspension in 1×PBS, pH 7.4, containing 5% (v/v) FBS. The resuspended cells were then plated in a 96-well plate in triplicate and immediately analyzed for fluorescence using a BD Accuri C6 Plus flow cytometer. Flow cytometry results were analyzed using FlowJo v10 software as specified previously. Each transfection experiment was performed in biological triplicates.

Confocal Microscopy. Cells were prepared for confocal microscopy by plating them on either an 8-chamber well slide (Millicel EZ Slide, Millipore Sigma, Burlington, MA) or 6-well plate with coverslip 16-24 hours before transfection. 8-chamber slides and coverslips removed from 6-well plates were mounted with Fluoroshield™ (Sigma-Aldrich, St. Louis, MO, USA). At timepoints of 0, 24, 48, and 72 hours post-transfections, cell media was aspirated, wells were washed with 1×PBS, pH 7.4 and then fixed using 4% (w/v) paraformaldehyde in 1×PBS, pH 7.4 and resuspended in 1×PBS, pH 7.4 for imaging. Confocal imaging was carried out using a Nikon A1R TIRF STORM Confocal microscope courtesy of the Cancer Center Microscopy Shared Resource at UC San Diego's Moore's Cancer Center.

### Results

PVX VLPs can encapsulate RNA cargos of different lengths. In vitro transcribed size-controlled mRNA transcripts, derived from PVX genomic RNA (gRNA) fragments cloned into the pCMV-T7-PVX-Genome plasmid, were designed as shown in FIG. 3A. gRNA fragments ranging from sizes of 100 bp-6.4 kb were generated by PCR amplification from the plasmid followed by IVT to yield pre-mRNA that was then poly(A)-tailed and capped. IVT of the 100 nt-long RNAs was low yielding (FIG. 3B). It was also apparent that, especially for the shorter RNAs, higher molecular weight bands were detected, resulting in a laddering effect in the size range of 1-5 kb—it is these contaminants that may have caused formation of PVX assemblies longer than predicted (see below).

For size-controlled VLP assembly and packaging of mRNA containing various length of gRNA fragments, purified CPs from PVX purified from N. benthamiana were used; re-assembly reactions were carried out using a 50:1 mass ratio of CP:RNA (or 4,200 CPs per mRNA molecule).⁷ The size distribution of the assembled VLPs was then determined from TEM images (FIG. 3C) taken at magnifications of 15 kX to 30 kX and quantified through standardized pixel-to-nanometer measurements using FIJI 2.9's image processing packages, using a minimum of 50 images and 130 particles per condition. This was plotted as histogram distributions of VLP measurements by frequency of size measurements that displayed a trend in increasing size by input mRNA size, and a sizable distribution shift when presented with mRNA of the same size as PVX's native gRNA (FIG. 3D-1E). Representative TEM images of each median VLP size displayed the characteristic filamentous morphology when packaging ≥1-knt mRNA templates; in contrast VLPs assembled on shorter templates, 250 nt and 500 nt mRNA, resembled more rigid rod-shaped structures (FIG. 3C).

When analyzing the size of the assembled VLPs containing mRNA cargos of various lengths, trends can be observed that implicate viral mechanisms of in vitro assembly that contrast with evolved in vivo assembly. PVX VLPs, when packaging native PVX gRNA, were measured at a median length of 616.6 nm—longer than the measured median length of native PVX virions at 504.8 nm (as shown in FIG. 3C-E)—despite the formation of particles using the same starting materials (wildtype CP and gRNA). This is likely due to less optimal RNA-packaging during in vitro assembly, leading to decreased packaging efficiency and stability of the VLP or nucleation of RNA-CP binding events that create hollow “tube ends” beyond the length of the packaged RNA. In principle template RNA should serve as a guide and constraint for VLP size, however when observing the other size conditions in our studies we could only note a trend for size-control. For example, VLPs packaging mRNA of 3-knts measured about half in length (median length: 331 nm) compared to VLPs with the native genome—this is as predicted. However, VLPs packaging mRNA 1.5 knt in length also displayed a median length of 314 nm. These discrepancies in size may be explained by the contamination of assembly reactions with larger RNA fragments (as observed by agarose gel electrophoresis, FIG. 3B), end-to-end or overlapping alignments of smaller genomic fragment mRNAs forming multiplets, and overextension of the CP nucleation mechanism leading to empty ends with genomic mRNA template.

PVX VLPs can assemble with mixed wildtype and His-tagged CPs. To streamline manufacture of the CP, we also produced the PVX CP in E. coli to complement the more specialized plant molecular farming with a more widely established technique. At the same time, we introduced a His-tag into the CP to enable purification but also to introduce a handle for functionalization: the His-tag allows for functionalization through coordination with Ni-NTA-appended cargos. In the future such system could be used to display targeting ligands (peptides, antibodies, aptamers) to achieve tissue-specificity.

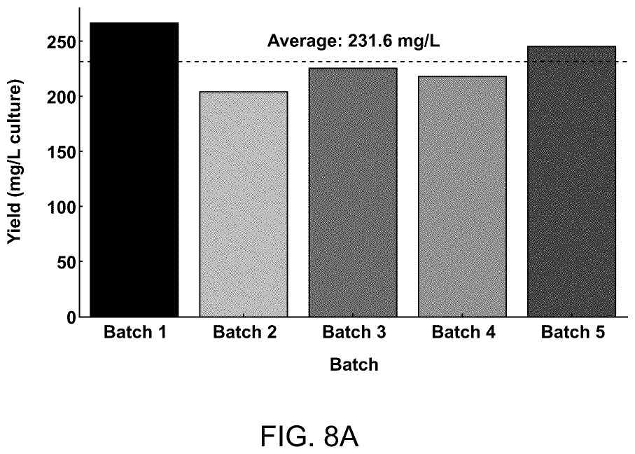

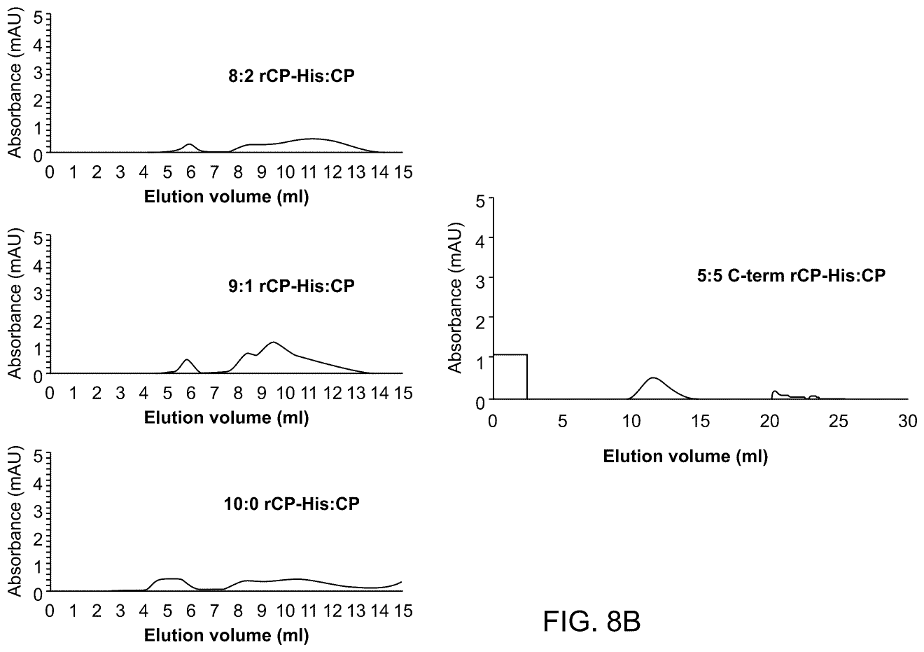

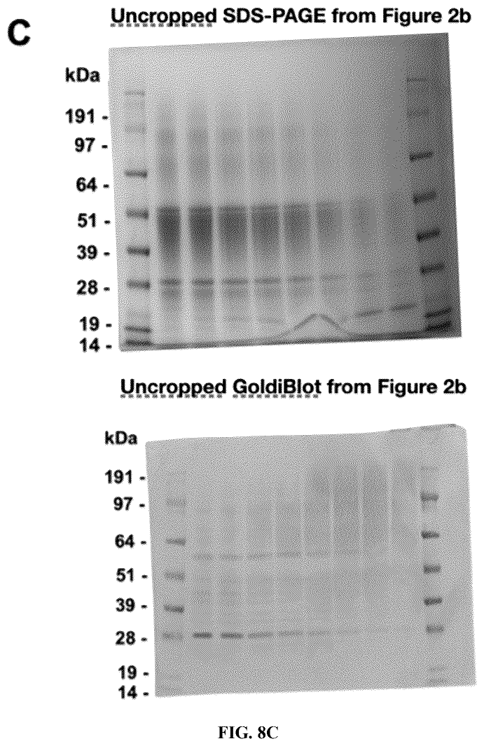

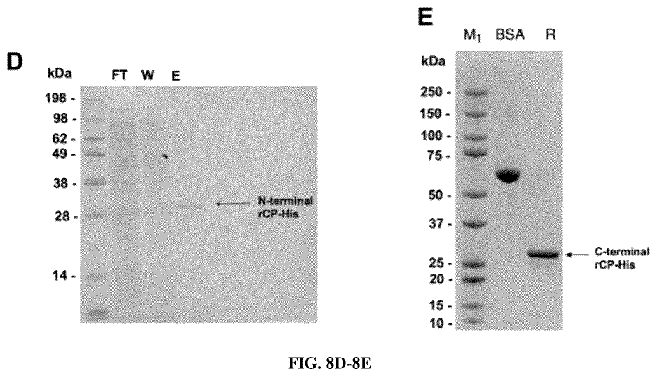

Recombinant CP-His (rCP-His) was produced at yields ranging from 204 to 265.5 mg per 1 L bacterial culture (FIG. 8A). While rCP-His protein alone did not yield VLPs when templated on genomic RNA—the addition of native CP enabled formation of VLPs containing a mixture of rCP-His and native CP. Applicant note that both, rCP-His with N-terminal or C-terminal His-tags could be expressed in and purified from E. coli, however the C-terminal His-tag—with or without the addition of native CP—did not yield VLPs (FIGS. 26B-26C); therefore N-terminal rCP-His should be considered.

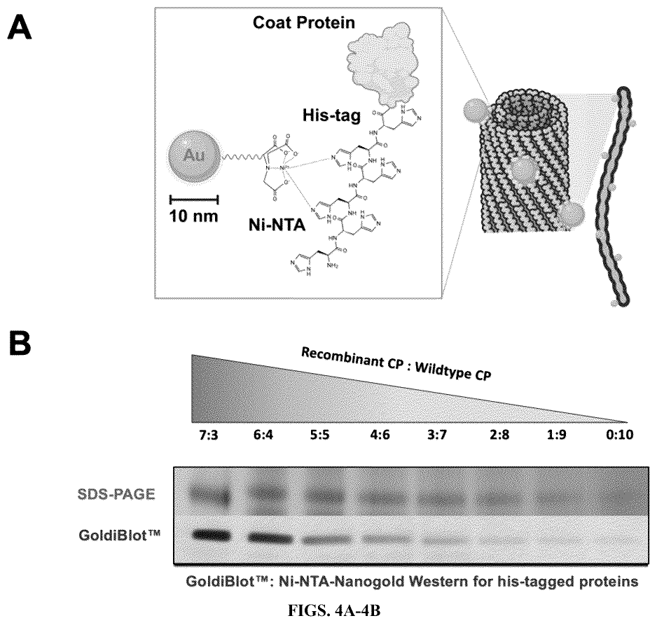

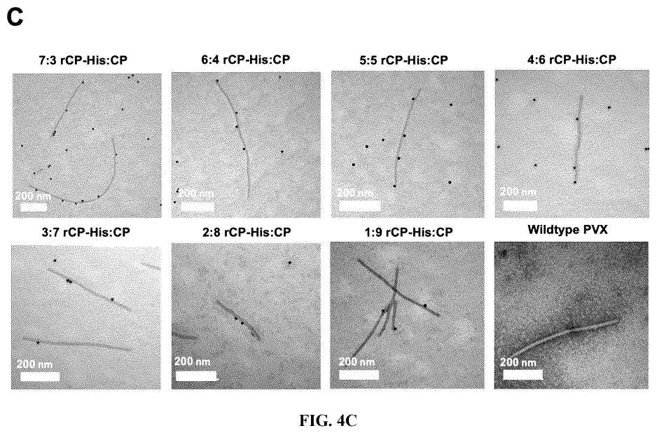

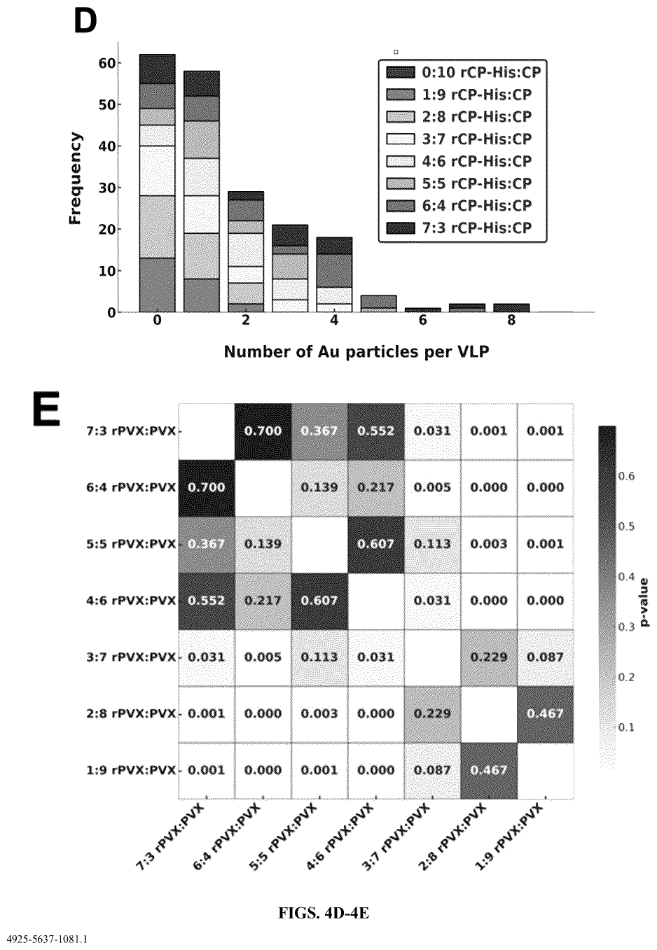

Native CP (obtained from PVX produced in plants, purified, and then disassembled) and rCP-His were mixed at varying ratios 7:3-0:10 rCP-His:CP; then mixed with genomic RNA purified from native PVX; the CP:RNA mass ratio was kept at 50:1. Mixed assembly reactions yielded VLPs with fidelity using ratios ranging from 7:3 rCP-His:CP to 1:9 rCP-His:CP, whereas ratios of 8:2, 9:1, and 10:0 rCP-His:CP were unable to reliably produce VLPs (FIG. 8D). This indicates that a certain degree of native CP is required to enable nucleoprotein assembly. To confirm the incorporation of rCP-His into the mixed assembly VLPs, 10 nm Ni-NTA-Nanogold® gold nanoparticles were used for detection in a Goldiblot™ western blot assay and immunogold TEM imaging (FIG. 4B-C). Goldiblot™ western results revealed increasing band intensity with VLP samples that were assembled from higher rCP-His:CP ratios, indicating that the addition of higher equivalents of rCP-His leads to higher incorporation of the rCP-His protein (FIG. 4B and FIG. 8E). The presence of rCP-His and the ability to functionalize the nucleoprotein assemblies via the tag was further validated using Nanogold® TEM (FIG. 4C). It should be noted that this method is not quantitative but serves to analyze trends. 130-210 VLPs per assembly condition were imaged at 15 kX to 60 kX and analyzed using FIJI 2.9's image processing packages, then plotted as a stacked bar chart (FIG. 4D). Data analysis indicated a trend: increased rCP-His added into the assembly mix correlated with a larger number of gold nanoparticles bound per VLP—thus data is in agreement with the western blot results. At the lower end of 1:9 and 2:8 rCP-His:CP VLPs displayed up to 2 gold nanoparticles only; at the 3:7 and 4:6 ratio up to 4 gold nanoparticles per VLP were counted; counts continued to increase with the 5:5 condition yielding up to 5 gold nanoparticles per VLP, 6:4 resulted in up to 7 gold nanoparticles per VLP, and with the 7:3 condition up to 8 gold nanoparticles per VLP were observed (FIG. 4D). To assess differences between the frequencies of gold nanoparticle per VLPs as a function of rCP-His:CP ratios, a Mann-Whiteney U Test was performed and visualized using a heatmap colorized by p-values (FIG. 4E). Strong statistical significance (p<0.001-0.1) was observed between the lower rCP-His:CP ratios as compared to the higher rCP-His:CP ratios, with lower significance observed between neighboring or similar conditions.

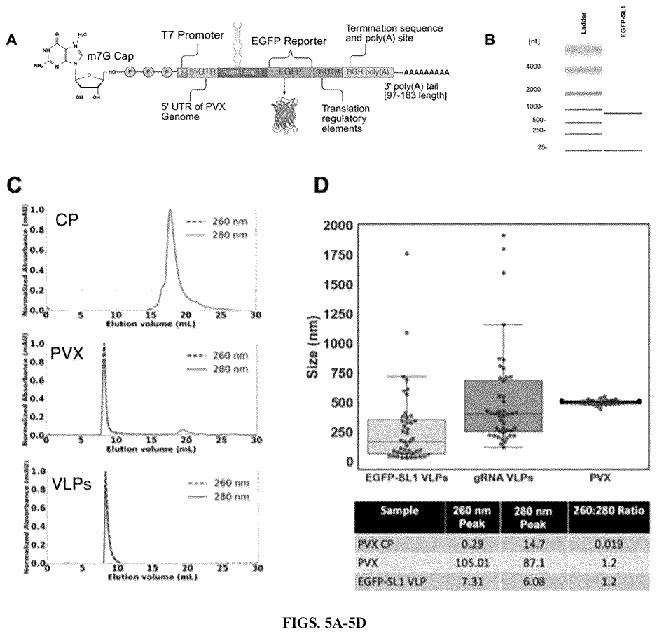

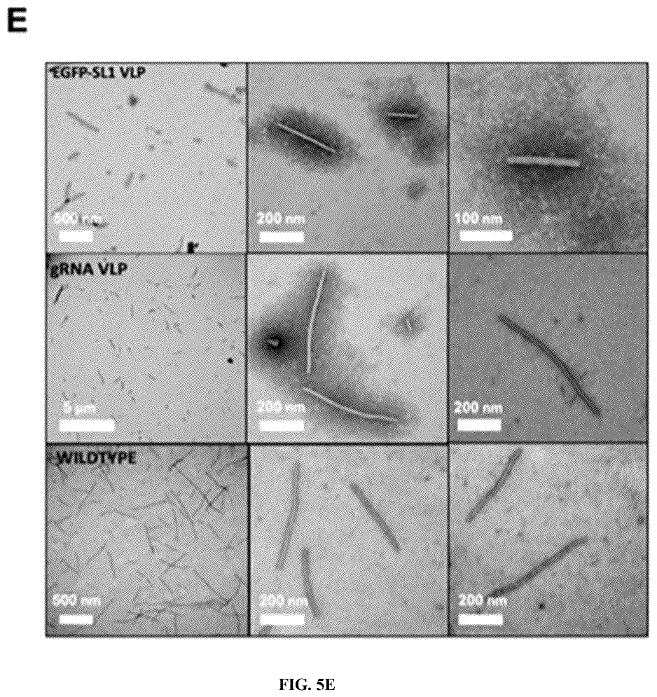

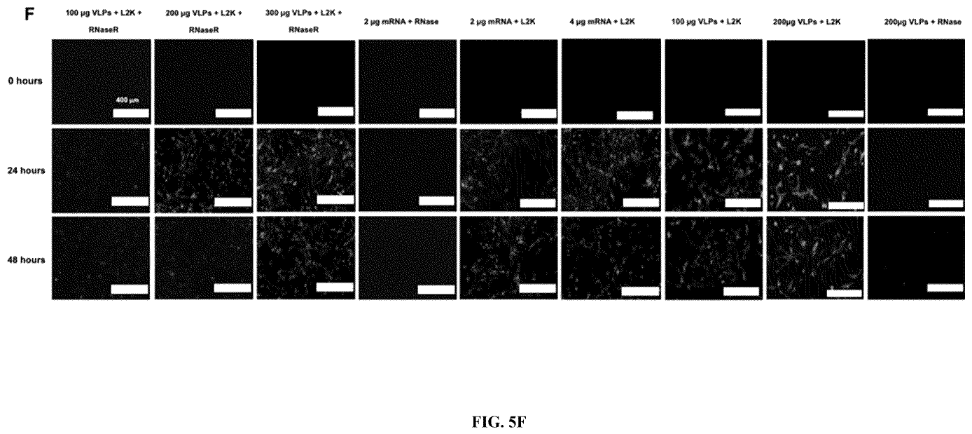

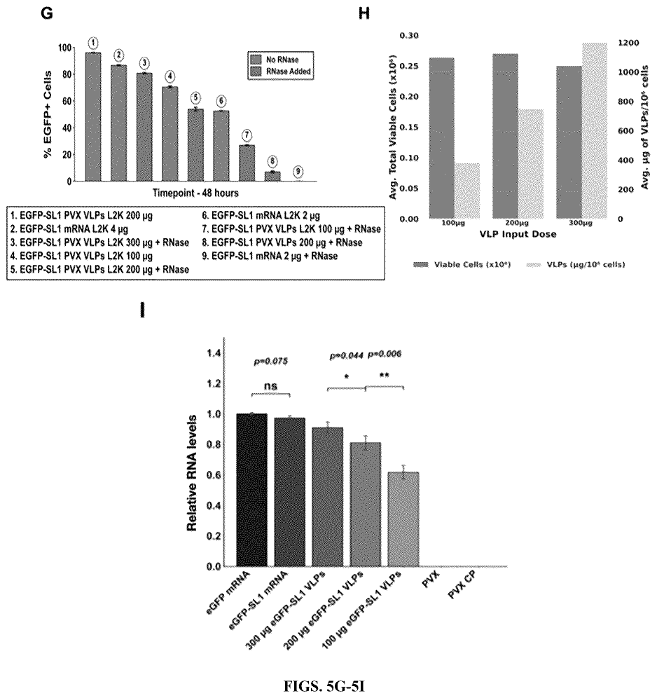

PVX VLPs are capable of packaging foreign IVT mRNAs for delivery and protein expression in mammalian cells. To assemble VLPs and package mRNA payloads for protein expression, CPs were isolated from PVX produced in plants and reassembled with either target mRNA or gRNA at a mass 50:1 ratio of CP:RNA. FIG. 5A depicts the mRNA coding for the reporter gene EGFP—the purity and size of the mRNA construct was confirmed by analysis using an Agilent 2100 bioanalyzer system (FIG. 5B).

SEC was used to determine the purity, size profile, and RNA encapsulation of VLPs in comparison to free CP and native PVX. Free PVX CP had a A260 nm:280 nm ratio of 0.02 while PVX and assembled EGFP-SL1 VLPs had a A260 nm: 280 nm ratio of 1.2—characteristic of intact nucleoprotein assemblies (FIG. 5C). The elution profile of EGFP-SL1 VLPs was comparable to that of native PVX—the slight shift from 8.27 ml elution volume for native PVX compared to 8.63 ml for EGFP-SL1 is consistent with the shorter length of the EGFP-SL1 vs. native PVX, indicates a significantly smaller particle size (FIG. 5C).

Size distributions of VLPs were also derived from TEM images taken at 15 kX magnification and quantified through standardized pixel-to-nanometer measurements using FIJI 2.9's image processing packages. EGFP-SL1 VLPs displayed an average size of 277.2 nm, a minimum size of 29.6 nm, a maximum size of 1,762 nm, and a median size of 169.3 nm (FIG. 5D). In comparison, PVX gRNA VLPs exhibited an average size of 519.3 nm, a minimum size of 122.2 nm, a maximum size of 1,917 nm, and a median size of 403.9 nm (FIG. 5D), demonstrating a size profile similar to native PVX (~485-525 nm; FIG. 5D). EGFP-SL1 VLPs with the 870 nt mRNA, in principle, should measure ~70 nm. The increased median size of 169.3 nm may be attributed to gRNA contamination, multiple or overlapping RNA transcripts incorporated into a single VLP, or overextended assembly and size-control experiments need further optimization (as discussed above). The morphology of EGFP-SL1 VLPs exhibited similar filamentous structures as compared to gRNA VLPs and native PVX (FIG. 5E). The diameters remained consistent at ~15 nm across all VLP and native PVX groups, while the aspect ratio remained large across all size conditions with the smallest being shorter EGFP-SL1 VLP particles (FIG. 5E). These findings suggest that the properties of assembled VLPs retain the original qualities found in native PVX despite the encapsulation of a foreign mRNA template containing an OAS structure.

The ability of EGFP-SL1 VLPs to deliver mRNA and express EGFP was then assessed through flow cytometry measurements using in BHK-21 cells (FIGS. 3F-G). Higher doses of VLPs transfected with Lipofectamine 2,000 exhibited the greatest % EGFP expression. When RNase was added the gene expression efficiency of the free mRNA constructs was greatly diminished—in stark contrast, packaging mRNA into PVX VLPs conferred protection of the mRNA from enzymatic degradation. Without RNase treatment the expression levels of mRNA-laden VLPs vs free mRNA were comparable (using lipofected and normalized mRNA amounts −200 g VLPs contain 4 g mRNA). However, when RNase treated, the VLPs outperformed (FIGS. 3F-3G). Finally, when unassisted by Lipofectamine, EGFP-SL1 VLPs exhibit modest and variable delivery efficiencies, which could be further enhanced through genetic and chemical modifications. It is of note, that in vitro plant VLPs have been shown to require lipofectamine, but in vivo lipofectamine is not required.¹⁶ This highlights the differences between in vitro and in vivo testing and will require follow-up studies to delineate these mechanistic differences. The mass of VLPs (in μg) per million cells for each condition was also quantified to showcase the relationship between dose-escalation and cell viability demonstrating low cytotoxicity of treated cells (FIG. 5H). Finally, we assayed the quality of the mRNA packaged in the VLPs: mRNA was isolated from disassembled VLPs and efficiency of cell transfection was assayed compared to free EGFP-SL1 mRNA. mRNA isolated from EGFP-SL1 VLPs was detected at levels comparable to that of free mRNA (FIG. 5I).

PVX CPs are Capable of Encapsulating circRNA to Form Halo-Shaped VLPs

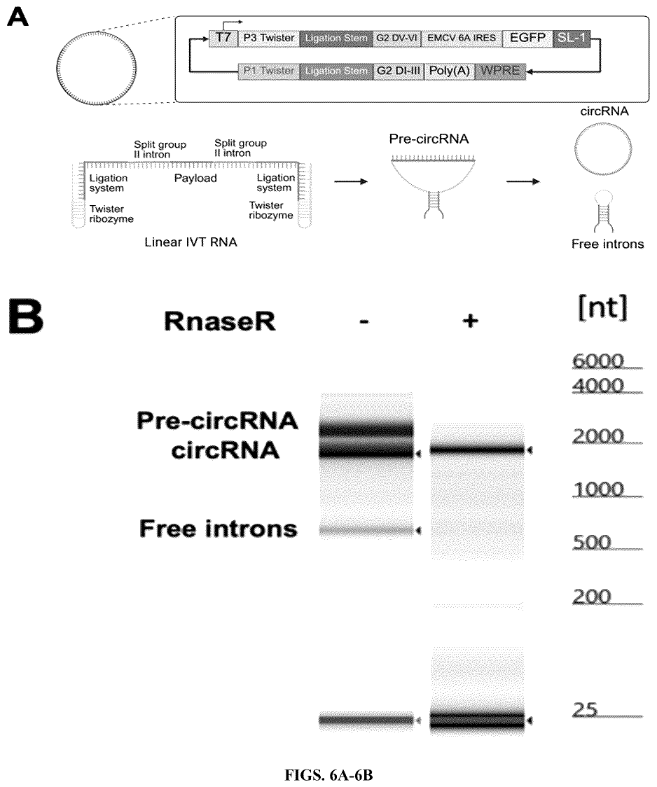

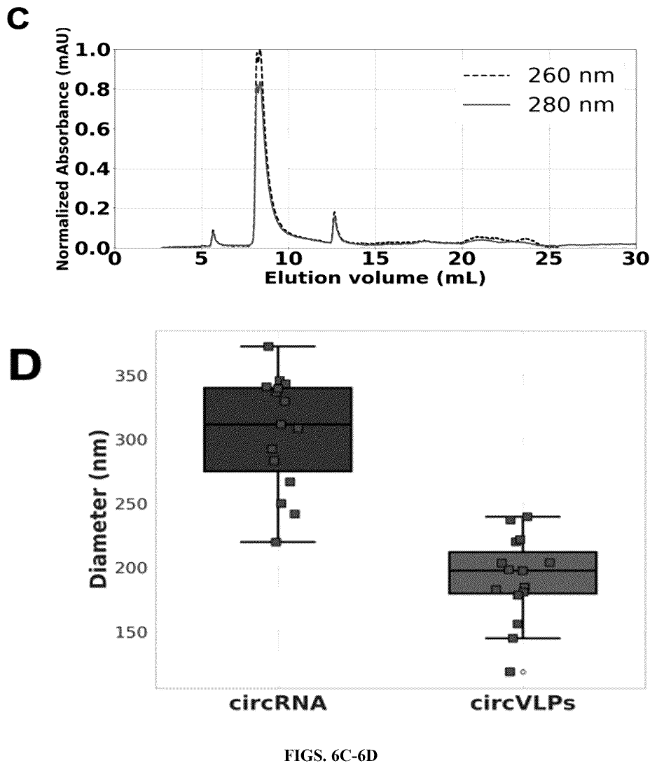

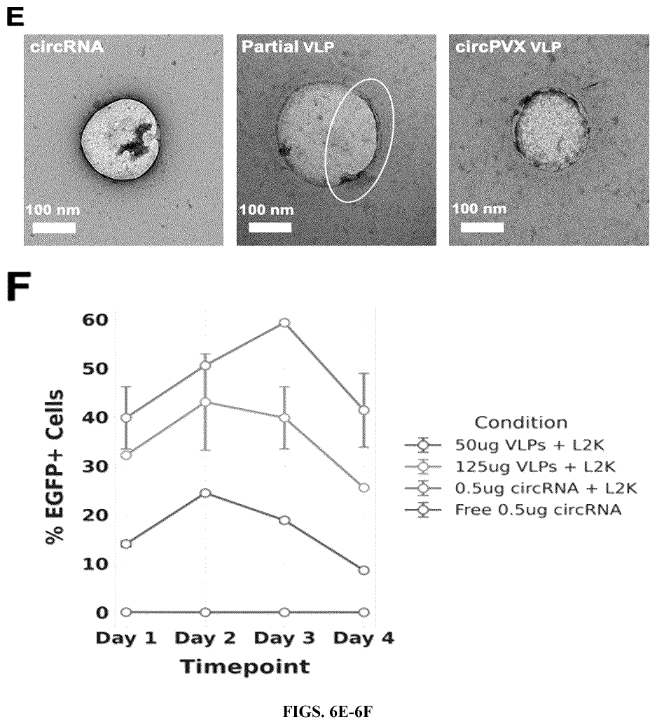

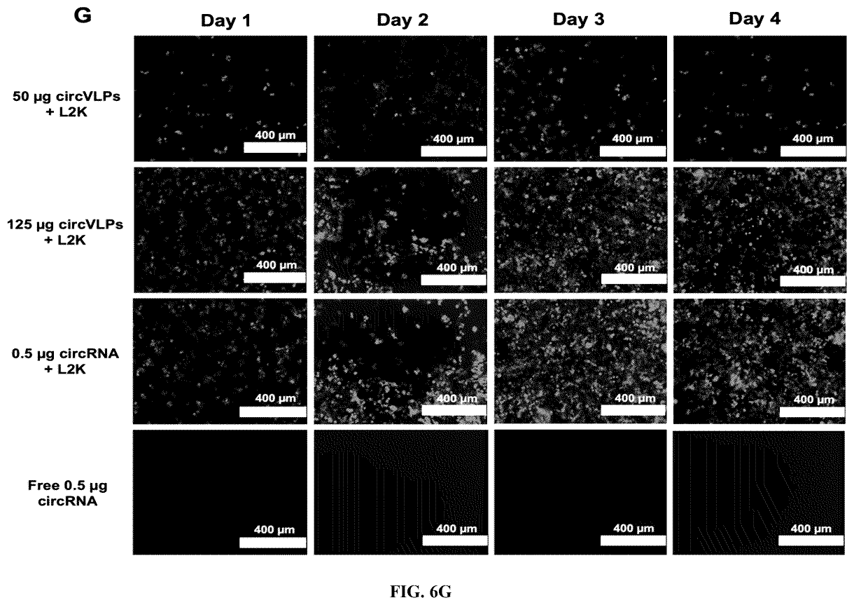

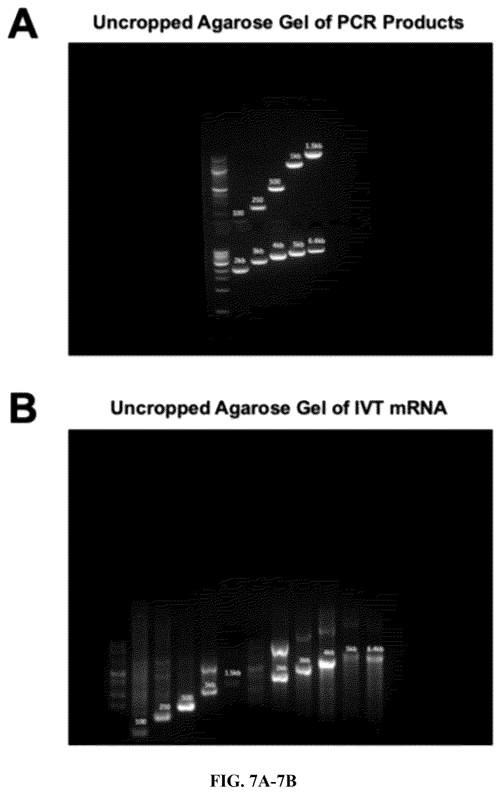

To test whether the PVX encapsulation and nucleation mechanism could incorporate other classes of RNA, a circRNA plasmid was adapted from ref.³³ The design, as shown in FIG. 6A—the host plasmid contains several features necessary for producing circRNA. It includes the group II intron from Clostridium tetani^{34,35,36} as previously described, where the I, II, and III domains were split apart from domains V and VI at domain IV to retain properties for intron splicing while allowing for RNA circularization. The domains of the intron were permuted to include domains V and VI at the 5′ end of the construct whereas domains I to III were included at the 3′ end. A minor leader sequence from domain DV/DVI group A was kept since it was likely to include the exon binding site (EBS) necessary for domain I intron-mediated selective splicing. To improved long-range interactions, flanking twister ribozymes that self-cleave at a high rate during IVT and allow for hybridization of complementary ligation stems to each other downstream. These circularization features are next to an internal ribosome entry site (IRES) paired with an EGFP reporter gene and a downstream PVX SL1 sequence before a 3′ UTR.

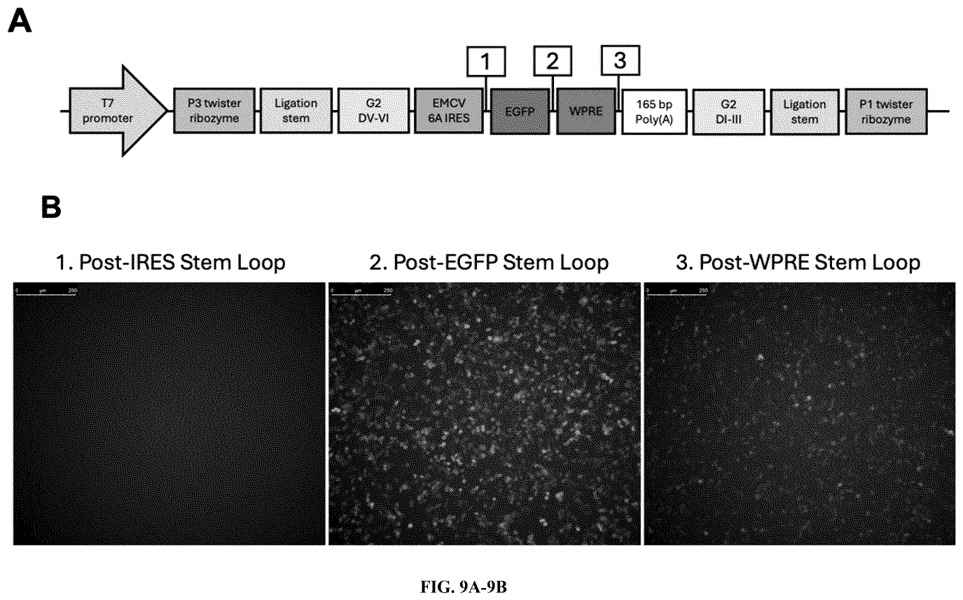

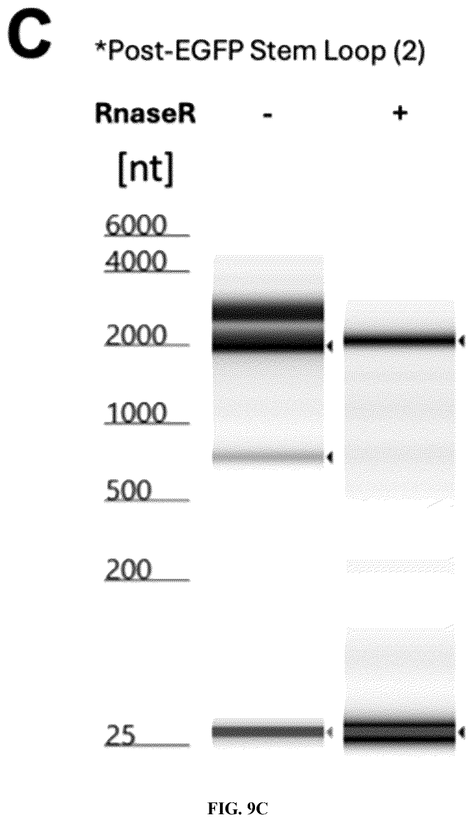

DNA templates were created through PCR followed by IVT of the RNA and its circularization. Three different sites were chosen to test optimal SL1 placement: post-IRES, post-EGFP, and post-WPRE, with the post-EGFP displaying good circularization rates and expression in HEK 293T cells (FIG. 9A-C) To characterize circRNAs and confirm circularization, a Bioanalyzer was used to visualize three species: pre-circRNA, circRNA, and free introns (FIG. 6B and FIG. 9C). Three bands can be observed in the absence of RNaseR, a nuclease that degrades linear RNA, and after treatment with RNaseR a single band representing the purified circRNA can be seen at 2,282 nt. To assemble circVLPs, purified CPs from PVX produced in plants were reassembled in vitro at a mass ratio of 40:1 CP:circRNA. SEC was used to determine the purity, size profile, and RNA encapsulation of VLPs (FIG. 6C). EGFP-SL1 circVLPs displayed a similar A260 nm:280 nm ratio of 1.2 to PVX, a tight distribution, and an elution volume of 8.41 ml (which comparable to the elution profile of native PVX, which eluted at 8.21 ml from the Superose6 Increase column); the delayed elution indicates a more compact size of the circVLPs vs native, linear PVX. While the shape of halo-PVX vs. native PVX is distinct, significant differences in SEC were not apparent, which is explained by the large size of the nucleoprotein assemblies eluting close to the void volume of the size-exclusion column.

To calculate the quantitative size distribution of circVLPs, in comparison to circRNA templates, diameter measurements were derived from TEM images taken at 15 kX-40 kX magnification and quantified through standardized pixel-to-nanometer measurements using FIJI 2.9's image processing packages (FIG. 6D-E). EGFP-SL1 circVLPs displayed an average diameter of 191.2 nm, a minimum diameter of 118.8 nm, a maximum diameter of 239.6 nm, and a median diameter of 197.58 nm (FIG. 6D). In comparison, circRNAs exhibited an average diameter of 305.53 nm, a minimum diameter of 219.8 nm, a maximum diameter of 372.55 nm, and a median diameter of 311.64 nm (FIG. 6D). Given that the contour length of RNA is 0.34 nm per base; the contour length of the PVX genome is ~2,000 nm which is packed into a ~500 nm-long nucleoprotein assembly—the condensation factor is 4. The circRNA of 2,282 nt would have a theoretical diameter of ~250 nm (measured 305 nm) and considering a condensation factor, the theoretical size of circVLPs would be <100 nm. Given the size was measured at 200 nm it is fair to assume that packaging of the RNA into the CPs is distinct. Nevertheless, the more compact nature of circVLPs vs. circRNA indicates that packaging indeed occurs. ‘Complete’ circVLP assemblies were observed with continuous negative staining—data also indicate that partial assemblies were formed where nucleation did not reach completion (FIG. 6D). This may be due to sub-optimal circRNA:CP mass ratio reactions, strength of the protein-protein and bond interactions of PVX CP during protein-RNA binding, or angular steric hindrance due to the presence of the SL1 RNA structure.

To assess the ability of EGFP-SL1 circVLPs to deliver circRNA to HEK 293T cells, flow cytometry and confocal microscopy were used to measure lipofected cells expressing EGFP over the course of 96 hours (4 days) (FIG. 6F-G). Due to the unavailability of a reliable nuclease for circRNA degradation, circVLPs were double sucrose-cushion purified before transfections. circVLPs demonstrated comparable transfection efficiencies as compared to the free EGFP-SL1 circRNA. Flow cytometry results were consistent with confocal microscopy results, showcasing average delivery efficiencies of the 125 μg circVLP were non-inferior to the circRNA positive control until the day 3 timepoint (FIG. 6F). The lower 50 μg circVLP condition also displayed relatively good levels of expression as compared to the positive and negative controls, demonstrating that the EGFP-SL1 mRNA cargo is intact and capable of translation even at lower dosages (FIG. 6F).

### Discussion

Applicant has demonstrated that the plant virus PVX and its CPs provide a versatile nanotechnology platform enabling packaging and delivery of RNA therapeutics. PVX VLPs can package diverse RNAs of various lengths (ranging up to 6.4 knts), sequence, and shape (linear mRNA vs. circRNA)—and the nucleoprotein assemblies can be formed from native or mixtures of native and His-tagged proteins thus providing a means for functionalization (e.g. in future work, targeting ligands could be introduced through complexation via Ni-NTA, similar to a previously reported study using PVX modified with TRAIL (a therapeutic protein engaging death receptors on tumor cells).³²

Concurrently, to enable engineering and future integration with cell targeting or additional therapeutic capabilities, mixed assembly VLPs were achieved by incorporating recombinant His-tags; the mixed assemblies retained their structural integrity when a minimum ratio of native CP:rCP-His of 3:7 was mixed. Applicant theorized that this is due to a cooperative binding of CP subunits that involves a minimum number of wildtype CPs (or untagged CPs) to contribute to the protein-protein and protein-RNA interactions that initiate nucleation. Here the His-tag was used to label the VLPs with gold, but the concepts could be expanded to incorporate therapeutic proteins and targeting ligands. Opportunity also exists to produce recombinant CPs with target functionality directly incorporated as N-terminal fusion. Optimization of the assembly conditions likely would be required considering the His-Tag is a rather small modifier (1.8 kDa). In combination with the inclusion of synthetic mRNAs, these mixed assembly VLPs could also be utilized as powerful RNA therapeutics technologies with dual effector actions, such as modifying the surface CPs with tumor-targeting ligands and encapsulating mRNAs encoding a therapeutic cytokine, where payloads and surface properties could be used to achieve a combined therapeutic aim.

Applicant has also shown the ability of PVX VLPs to encapsulate synthetic foreign mRNAs of different classes—specifically linear and circRNAs—highlighting the platform's adaptable nature. These studies feature the versatility of PVX as a flexible nucleoprotein assembly technology—so flexible indeed that the assembly of halo-shaped PVX packaging circRNA was achieved. Cell transfection using PVX VLPs packaging linear mRNA or circRNA was accomplished and were non-inferior to control RNA conditions. Additionally, the high dosage of VLPs per cell in culture corresponds to a lower cytotoxicity, higher biocompatibility, and a promising safety profile.

### Example 2: Design of Additional Filamentous mRNA-Laden PVX and the Design and Synthesis of a mRNA Cassette

For in vitro transcription of the mRNA cassettes, the synthetic gene can be cloned into a transcription plasmid, for example IDT Bluescript under control of a T7. The plasmid will be amplified in E. coli and transcribed using available kits, e.g. MEGAscript T7 Transcription Kit (Thermo Fisher). To achieve efficient translation in the target cell, regulatory elements can be added. A polyA tail can be included in the sequence and the 5′Cap can be appended either post in vitro transcription using capping enzymes (e.g., New England Biolabs). It is also possible to obtain capped mRNA by transcription through addition of the dinucleotide m7G(5′)-ppp-(5′)G. Applicant can also incorporate the PVX origin of assembly site (OAS) to prime coat protein assembly on the target RNA.

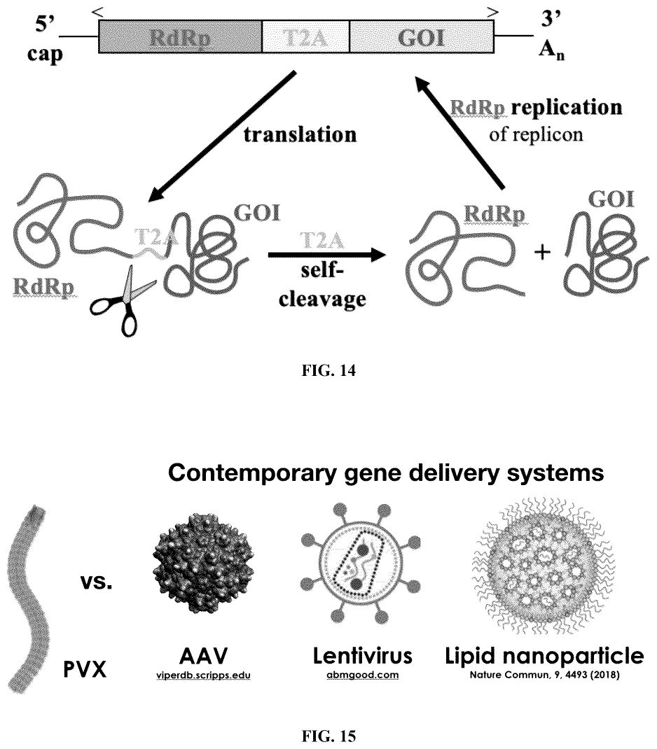

Optionally, the mRNA cassette is designed to be self-amplifying. Applicant designed self-amplifying mRNA cassettes. That is, a cassette is designed to deliver the mRNA in replicon form (FIG. 14). While non-integrating vectors are safer, the downside is the shortened therapeutic window, requiring frequent redosing. This downside is overcome by delivering therapeutic sequences in the form of self-replicating RNA replicons. Specifically, coding sequences of the Nodamura virus or Sindbis virus RNA-dependent RNA polymerase (RdRp) are incorporated into the mRNA cassette. The gene of interest is separated from the RdRp by a self-cleaving peptide, so that straight-through translation yields a polyprotein that self-cleaves to give functional RdRp and gene of interest (for example EGFP, GM-CSF, or IL2), with the RdRp then strongly amplifying the overall replicon and hence its gene products. The replicon can lead to longevity of the mRNA in cells resulting in higher level of protein expression in transfected cells. These concepts allow to design a material that is non-integrating but with enhanced therapeutic window.

Once the design concepts are established, candidate materials could be scaled up by plant molecular farming. Applicant can make use of Agrobacterium tumefaciens and use a binary shuttle vector; N. benthamiana plants can serve as production species. RNA loading into the PVX vector could be achieved through co-expression of the target RNA sequence and the desired coat protein. The design principles described herein have been experimentally validated using genomic RNA fragments of varying length, demonstrating the relationship of nucleic acid size with PVX particle length.

### Example 3: Design of Ring-Shaped circPVX Templated on circRNA

This example describes exemplary viral-based gene delivery vectors comprising a ring-shaped PVX nanoparticle (circPVX) as a novel nanotechnology tailored for delivery of circRNA.

Design and Synthesis of a circRNA Cassette

To generate the circRNA for e.g. EGFP expression, a plasmid with T7 promotor for in vitro transcription is designed to contain the following elements: the EGFP coding sequence can act like an exon; the exon is flanked by intronic sequences that facilitate backsplicing, resulting in the formation of the circRNA. Several circRNA studies have identified intronic motifs, such as Alu repeats, that promote circularization. To enable cap-independent expression an internal ribosome entry site (IRES) are included. Finally, the circRNA can contain the PVX OAS to drive coat protein binding and initiate assembly. circRNAs are generated from precursor mRNA (pre-mRNA) by a process called backsplicing. The pre-mRNA can be obtained through in vitro transcription. The DNA templates are removed by DNase treatment and the synthesized RNA id purified. Applicant then applies heat-cool cycles to facilitate the back splicing and circularization of RNA. Heat opens up the RNA structure, and as it cools down, the RNA tends to fold to facilitate backsplicing to form circRNA. During the process of splicing events, the 3′ hydroxyl group of the guanosine nucleotide initiates a transesterification reaction at the 5′ splice site. The 5′ upstream section of the intron is excised, and the released hydroxyl group at the end of the intermediate engages in a second transesterification at the 3′ splice site, resulting in circularization of the intervening region and excision of the 3′ intron. Finally, Applicant can treat with RNase R, an exonuclease that digests linear RNA but not circRNA, to enrich the sample for circRNA. Product verification can be by RT-PCR using divergent primers to verify the formation of circRNA and sequencing. circRNA vs. linear RNA can also be analyzed by electrophoretic mobility shift assays to further validate that indeed circRNA is obtained.

Optionally, the circRNA cassette is designed to be self-amplifying RNA cassettes. That is, a cassette is designed to deliver the mRNA in replicon form (FIG. 14). Specifically, Applicant incorporates coding sequences of the Nodamura virus or Sindbis virus RNA-dependent RNA polymerase (RdRp). The gene of interest is separated from the RdRp by a self-cleaving peptide, so that straight-through translation yields a polyprotein that self-cleaves to give functional RdRp and gene of interest (for example EGFP, GM-CSF, or IL2), with the RdRp then strongly amplifying the overall replicon and hence its gene products. The replicon can lead to longevity of the circRNA in cells resulting in higher level of protein expression in transfected cells.

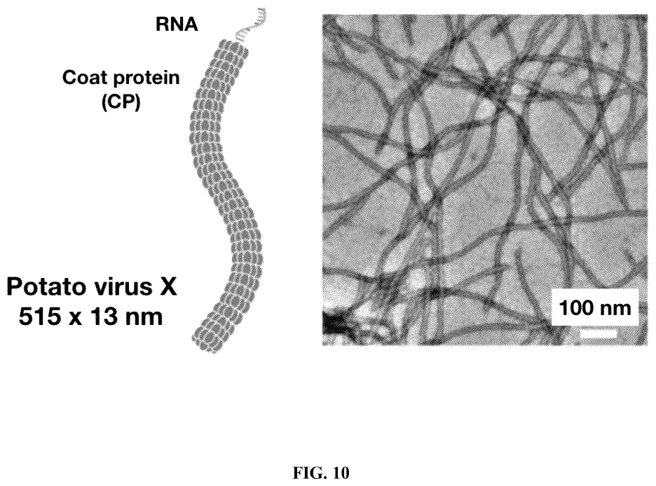

In vitro packaging of circRNA using the PVX coat protein. Purified PVX coat protein can be obtained and assembled on the circRNA template and can be produced in Nicotiana benthamiana using established protocols. For in vitro assembly, PVX is purified and disassembled into their coat protein units followed by re-assembly around the nucleic acid cargo of interest; these methods are already well-established for viruses and these methods can be adapted for PVX. To confirm structural integrity of the produced viral vectors, transmission electron microscopy is performed (TEM, see FIG. 10) and fast protein liquid chromatography (FPLC, FIG. 11). Lastly protein gel electrophoresis and western blots are used to confirm the presence of the PVX-specific coat proteins and RT PCR and gel electrophoresis is used to confirm and quantify RNA encapsulation. Applicant anticipates that careful optimization of the following parameters is required: ratio of coat protein to circRNA, addition of coat protein in bolus vs. sequential micro dosing, incubation time and temperature, as well as bathing conditions (pH, salt, buffer).

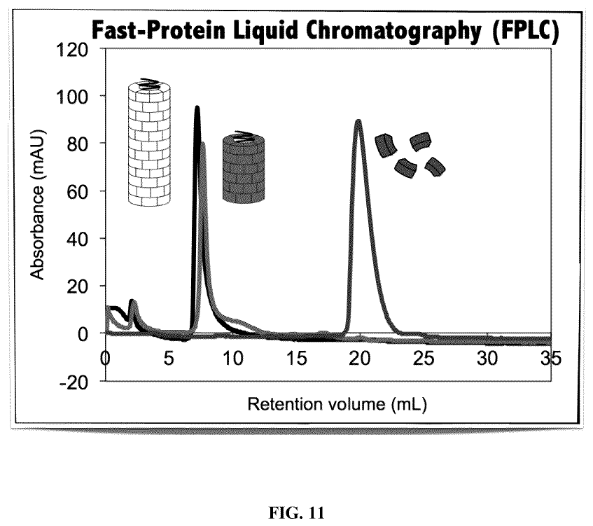

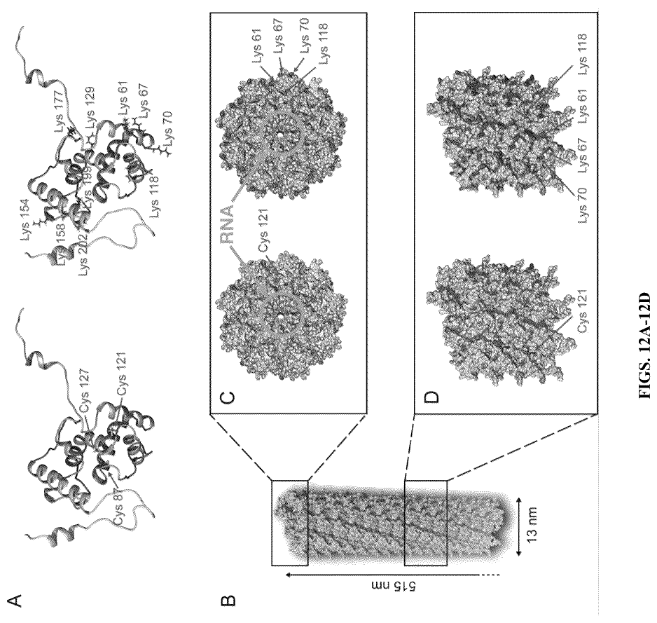

Tailoring the material properties of the nanoparticle: size of the nanoparticle. To take a deeper dive into the role of particle aspect ratio (size), Applicant makes use of the unique features of viral self-assembly to produce PVX filaments of distinct length (100 nm, 250 nm, and 515 nm×13 nm). Native PVX filaments measure 515×13 nm, and it is the RNA that defines the length of the nucleoprotein assembly—using different sized RNAs, PVX of defined length is obtained. To tailor the mechanical properties, Applicant can use a crosslinking approach: PVX offers solvent-addressable Lys and Cys chains (see FIG. 12). These sites are targeted with NHS- or maleimide-reactive PEG crosslinkers (e.g. NanoCS). Applicant can test X-PEGn-X crosslinkers, with X being NHS or maleimide, n=1-100 (or MW of 0.6-5 k Da), and number of branchesf=1 (bivalent), 4, and 8 (multi-arm); PVX of defined length are used. The use of multi-arm PEGs allows the crosslinking of multiple adjacent coat proteins.

The distance between two adjacent Lys/Cys is ~4 nm; hence Applicant can determine a suitable size cut-off (i.e., Flory radius) to enable interlinking of coat proteins. Further, Applicant can moderate the contour length to avoid a masking effect. That is, if the polymer chain is too long, the reactive groups could become buried due to the extrasteric hindrance. Using sequential and combinatorial labeling of Lys and Cys side chains is expected to result in highly interlinked nanoparticle system. Successful PEG conjugation and the degree of crosslinking is determined using a combination of SDS-PAGE and mass spectrometry. Without wishing to be bound by any particular theory, Applicant hypothesizes that multi-point crosslinking can result in changes in the mechanical properties and increased stiffness. Transmission Electron Microspy (TEM) imaging could provide insight into the mechanical properties as the persistence length might change upon crosslinking.

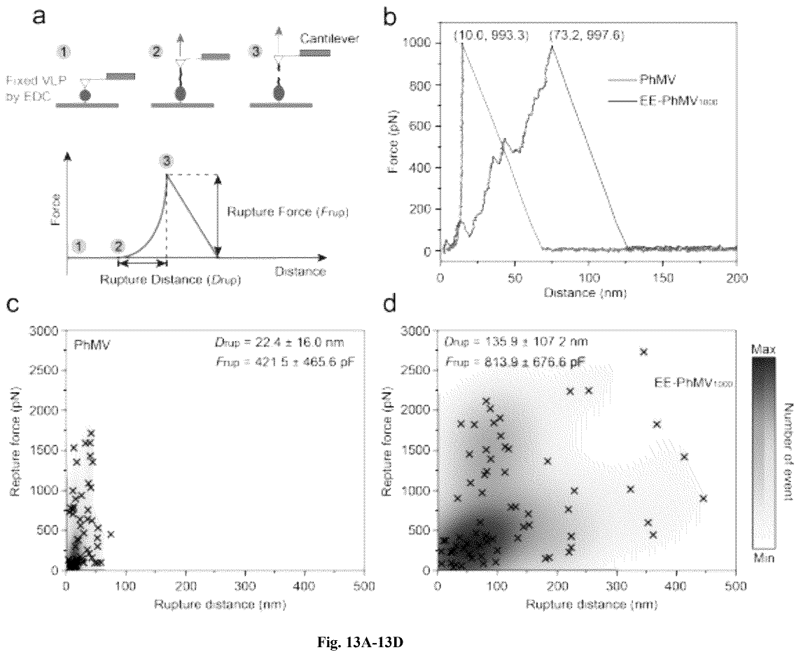

Mapping the mechanical performance of the nanoparticles. To map the mechanical performance single-molecule force spectroscopy (SMFS) using atomic force microscopy (AFM) can be used. Applicant recently applied this method to probe the analyze a spherical VLP internally crosslinked via PEG linkers. Indeed, the crosslinking strategies enhanced the mechanical performance when comparing the crosslinked vs. native VLP (FIG. 13). Here, Applicant can immobilize PVX on a substrate and then chemically couple it to an AFM cantilever. Differences in the SMFS force-extension curves can be attributed to the mechanical properties. Applicant can extract the rupture force (Fᵣᵤₚ) and rupture distance (Dᵣᵤₚ), with Dᵣᵤₚ being the change in tip substrate separation at which the force returns to zero following either rupture of the particle, detachment of the particle, or other scission event along the analyte. This methodology can therefore capture any difference in mechanical extension of the VLP that might be facilitated by unfolding of the inter-connected coat proteins. A single, smooth SMFS curve reflects an extension and rupture event with no evidence of multi domain protein unfolding. A sawtooth like curve however is consistent with a series of multiple mechanical rupture events.

Nanoindentation experiments are carried out to catalogue the Young's moduli of the designed PVX-based materials.

Example 4: Structure-function studies investigating the transduction efficiency of PVX vectors delivering circRNA. The particles evaluated herein were produced according to the methods in Examples 1-3, encompassing linear mRNA, circRNA, and mixed assembly variants.

The transduction efficiency of the PVX vectors (in presence/absence of anti-PVX antibodies) can be assayed and benchmarked against viral and lipid nanoparticle systems. This could be the first example of a ring-shaped nanoparticle being evaluated in cell culture.

Without being bound by theory, it is anticipated that integration of replicons can significantly enhance target protein expression; the amplification is expected to outperform synthetic systems and it might achieve matching viral vector efficiency, while providing safety. Without being bound by theory, Applicant hypothesizes that to be effective for nucleic acid delivery, cytoplasmatic mRNA delivery can be a key requirement.

Determine shelf life and stability of the plant viral vector. Some synthetic systems can experience instability in biological media and aggregate based on interactions with serum components. To determine the shelf life of circRNA-laden PVX, it is stored at −80° C., −20° C., 4° C., and room temperature for 1, 3, 6, and 12 months; nanoparticle stability and structural integrity are verified by TEM imaging, western blots, and RT-PCR to confirm the protein and RNA composition. One set of samples can also undergo freeze-thaw cycles. To confirm the stability in biological media, PVX is exposed to PBS, physiological saline (0.15 M NaCl) and cell growth medium containing 10% (v/v) serum, human plasma and/or serum (Sigma Aldrich). Applicant measures agglomeration and precipitation; dynamic light scattering is used to determine the hydrodynamic radius of the particles over time. Applicant assays stability with and without RNase added to the various bathing conditions and confirm whether the mRNA is indeed resistant to nuclease digestion.

PVX transduction efficiency assaying EGFP and GM-CSF/IL2 expression in various cell types. Without wishing to be bound by any particular theory, targeting the tumor microenvironment can restore anti-tumor immunity; therefore, Applicant chose macrophages, cancer cells, and fibroblasts as a testbed: RAW 264.7 mouse macrophages and SC human peripheral blood macrophages; MDA-MB231 and SK-BR-3, human triple negative and HER2+ breast cancer cell lines; 3T3 mouse or Malme-3 human fibroblast cell lines. These cell lines are available from ATCC.

Dose-dependency and time course studies to determine the transduction efficiency. Applicant first determines copy number and longevity of the mRNA constructs delivered by PVX in the target cells: time course studies are performed and mRNA and replicons are quantified 8, 24, 48 and 120 hours post transfection using RNA purified from whole cell lysates and quantitative RT-qPCR assay. Next, the transduction efficiency is determined. For EGFP, expression levels are quantified based on its fluorescence using flow cytometry and fluorescence imaging. GM-CSF or IL2 expression are quantified using specific antibodies and flow cytometry as well as quantitative western blots and ELISA methods. Lastly, A GM-CSF and IL2 bioassay are used to determine whether the cytokines are biologically active. For IL2, commercially available kits are used (e.g., Promega IL2 bioassay kit). For GM-CSF, bioactivity is assayed using TF-1 cells (ATCC), an erythrocyte cell line that is completely dependent on GM-CSF (and IL3). Cell proliferation is assayed using MTT assay (commercially available) in the presence of GM-CSF (positive control) or supernatant collected from cells transfected with PVX vectors.

Efficacy of the gene delivery system in the presence of anti-PVX antibodies. The role of antibodies in biologics clearance, cell uptake, and potential blocking of function are important factor for consideration for any biologic development and can be used in the early stages of development. This can allow for technology refinement; for example, if needed, PEG coatings can be applied, as is done with the synthetic nanoparticles (e.g., Moderna's and Pfizer-BioNTech COVID19 vaccines).

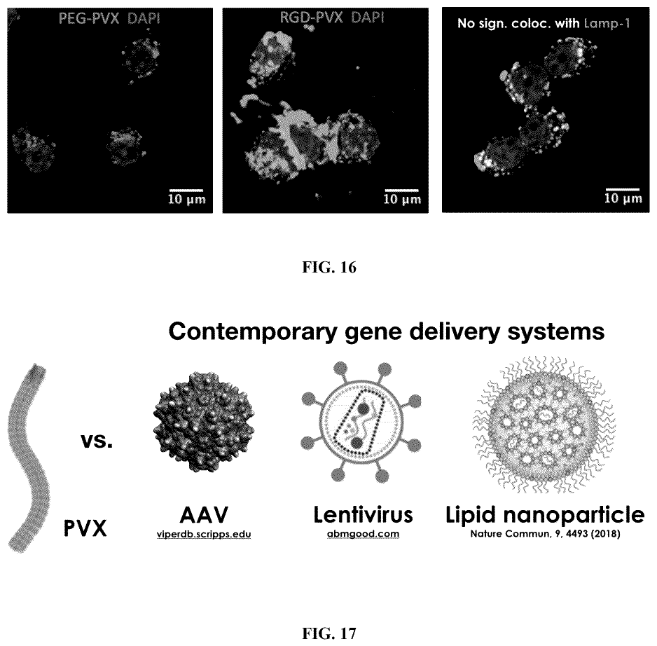

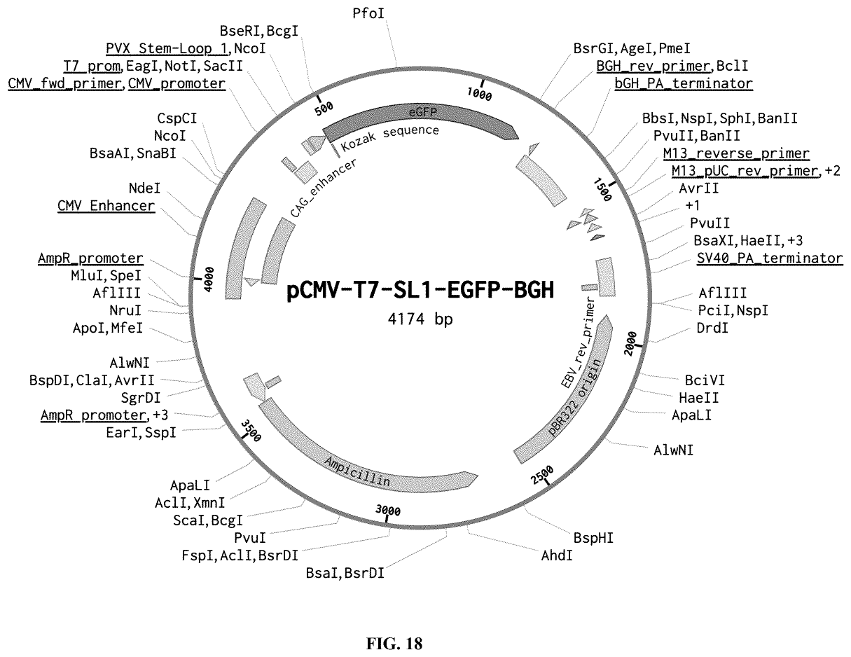

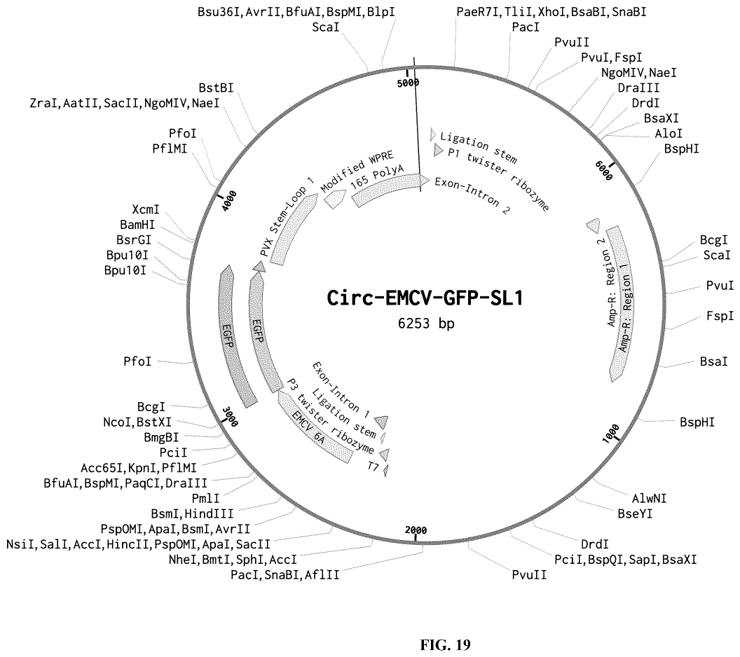

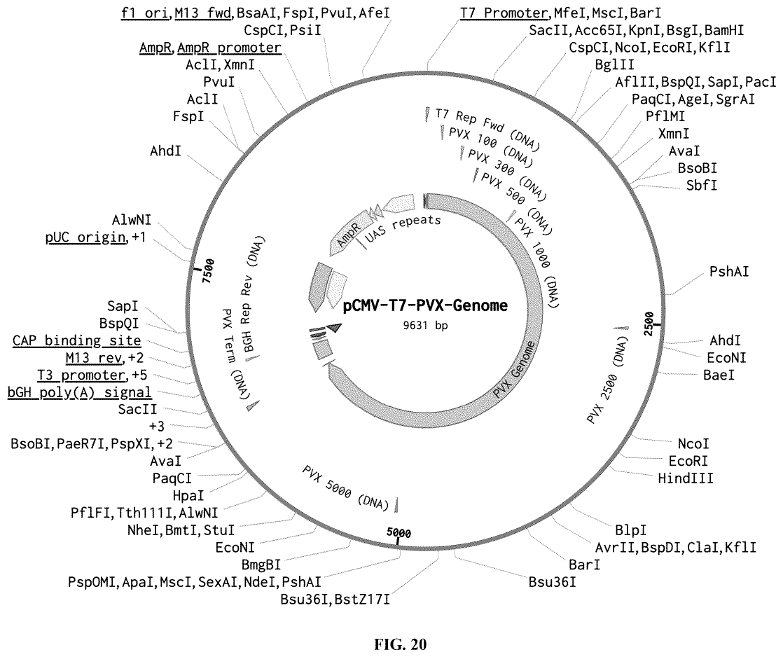

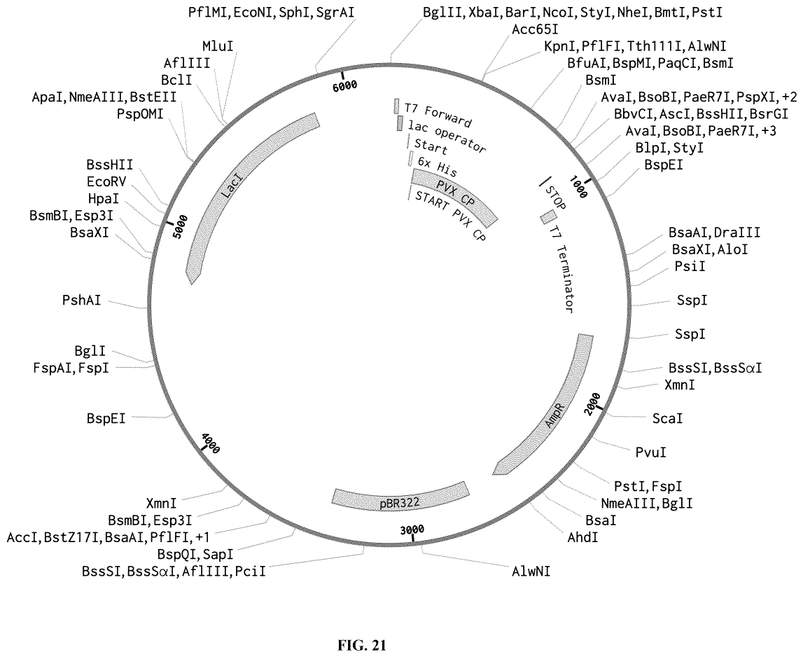

Benchmark experiments to compare longevity of the gene of interest among different systems. (FIG. 17). Adeno-associated virus (AAV) vectors as well as a Lentiviral system is used and can be obtained from commercial sources such as Clonetech, Vector Biolabs, or ABM. Lipid-based nanoparticles based are synthesized using methods known in the art; for example, recent protocols using mixtures of DSPC, Cholesterol, DMG PEG, and DSPE-PEG (Avanti Polar Lipids). Lastly, Applicant can also use commercial in vitro transcribed (IVT) RNA as control (e.g., Aldevron, Eurofins, ApeXBio), either transfected using lipofectamine or delivered using lipid-based nanoparticles. Efficiency and longevity of gene of interest expression using the methods as outlined above are used.

Monitor update of PVX into cell and PVX trafficking. Data indicate that plant viruses can indeed deliver nucleic acids to the cytosol of cells; for example expression of model antigens and fluorescent proteins using CCMV and TMV was demonstrated. Consistently, preliminary data indicate that PVX is not trapped inside the endolysosomal compartment (see FIG. 16). Nevertheless, studies detailing the trafficking of plant viruses in mammalian cells are lacking and to fill this gap in knowledge as well as to inform the efficacy studies, Applicant details how PVX interacts with cells (macrophages, cancer cells, and fibroblasts). Structure-function studies are performed, and assay cell uptake and trafficking as a function of aspect ratio and stiffness of the PVX gene delivery vectors are measured.

PVX-cell entry. Applicant has demonstrated that PVX enters mammalian cells, including cancer cells, macrophages, and fibroblasts. The role of specific endocytotic pathways is determined and cells are treated with known biochemical inhibitors of clathrin- or caveolin-mediated endocytosis and micropinocytosis (Table 2). Quantitative data is obtained using flow cytometry protocols: binding kinetics will be determined in dependence of concentration (105-8 PVX particles/cell) and time (minutes-24-hours); and this is complemented with confocal imaging. The studies of cellular entry mechanisms inform the intracellular trafficking.

TABLE 2
Cell entry studies

| Inhibitor | Pathway |
|---|---|
| NaN₃/2-deoxyglucose | energy-dependent cell uptake |
| Cytochalasin D | micropinocytosis and phagocytosis |
| Dynasore or chlorpromazine | clathrin-mediated endocytosis |
| genistein | caveolae-mediated endocytosis |

PVX-cell trafficking. Live cell imaging using confocal microscopy as well as co-localization studies using organelle-specific antibodies (available from Invitro gen) are carried out. To enable longitudinal tracking and imaging, cyanine or AlexaFluor dyes are conjugated to PVX using well established chemistries. Applicant determines whether plant viral-based materials are recycled and transcytosed or digested and metabolized.

Cell compartments are imaged, including the cell membrane (using for example wheat germ agglutinin staining), vesicles (using for example EEA-1 or Lamp-1 antibodies for early endosomes and endolysosomes), and nucleus (using for example 4′,6-diamidino-2-phenylindole (DAPI)). Further, quantitative western blots using virus-specific antibodies are conducted on cell extracts to evaluate if and when the protein shell is broken down. TEM imaging of ultrathin cryosections and immunogold staining provide further insights into the localization and intactness of the carriers over time. Further still, fluorescence in situ hybridization (FISH) is carried out to determine the localization of the viral RNA inside the cell. For example, detection of the mRNA at the ribosome-rich endoplasmatic reticulum would indicate protein translation. Finally, Applicant also stains for protein products including GM-CSF and IL2, or directly image EGFP.

The use of membrane active, pH-sensitive, fusogenic peptides to facilitate enhances escape is used if a majority of the PVX particles are trapped within vesicles.

The bioengineered ring-shaped filamentous plant virus nanoparticles that encapsulate designed circRNA cassettes, with size control, mechanical reinforcement, and optional surface targeting, enable the delivery of stable and functional nucleic acid therapies with the ability to add other therapeutic agents for synergistic effects.

### Sequences

TABLE 3
gBlocks

| | |
|---|---|
| SL1 cloned into pCMV- | TTTAGTGAACCGTCAGATCCGCTAGAGATCCGCGGCCG |
| T7-EGFP | CTAATACGACTCACTATAGGGAGAGCCTTGTTACACAC |
| | CCGCTTGAAAAAGCAAGTCTGACAAAAGGCCAAAGTG |
| | CGCGAGGGCCACCATGGTGAGCAAGGGCGAGGAGCTG |
| | TTCACCGGGGTGGTGCC |
| SL1 cloned into Circ- | GTGCTGGTTATTGTGCTGTCTCATCATTTTGGCAAAGC |
| ECMV-GFP | GGCCGCGGCCAGTGAATTGTAATACGACTCACTATAGG |
| | GGTTAGAGCCTTGTTACACACCCGCTTGAAAAAGCAAG |
| | TCTGACAAAAGGCCAAAGTGCGCGAGGGCCACCGGTA |
| | ACAAAAAAAAAAAAAAAAAAAAAAAATCACCGACCG |
| | ATCTATCTGAACCGGTGGGAATTCTAGAG |
| PVX Genome Segment 1 | TAATACGACTCACTATAagagccGAAAACTAAACCATAC |
| cloned into pCMV-T7 | ACCACCAACACAACCAAACCCACCACGCCCAATTGTTA |
| 1 nt-2131 nt | CACACCCGCTTGGAAAAGCAAGTCTAACAAATGGCCA |
| of the PVX | AAGTGCGCGAGGTTTACCAATCCTTTACAGACTCCACC |
| Genome | ACAAAAACTCTCATCCAAGATGAGGCTTATAGAAATAT |
| | TCGCCCCATCATGGAAAAACACAAACTAGCTAACCCTT |
| | ACGCTCAAACGGTTGAAGCGGCTAATGATCTAGAGGG |
| | GTTCGGCATAGCCACCAATCCCTATAGCATTGAATTGC |
| | ATACACATGCAGCCGCTAAGACCATAGAGAATAAACT |
| | TCTAGAGGTGCTTGGTTCCATCCTACCACAAGAACCTG |
| | TTACATTTATGTTCCTTAAACCCAGGAAGCTAAACTAC |
| | ATGAGAAGAAACCCGCGGATCAAGGACATTTTCCACA |
| | ATGTTGCCATTGAACCGAGAGACGTAGCAAGGTACCC |
| | CAAGGAAACAATAATTGACAAACTCACAGAGATCACA |
| | ACGGAAACAGCATACATTAGTGATACTCTGCACTTCTT |
| | GGATCCGAGCTACATAGTGGAGACATTCCAAAACTGC |
| | CCAAAACTGCAAACATTGTATGCGACCTTAGTTCTCCC |
| | CGTTGAGGCAGCCTTCAAAATGGAAAGCACTCACCCG |
| | AACATATACAGCCTCAAATACTTCGGAGATGGTTTCCA |
| | GTATATACCAGGCAACCATGGTGGTGGGGCATACCATC |
| | ATGAATTCGCTCATTTACAATGGCTCAAAGTGGGAAAG |
| | ATCAAGTGGAGGGACCCCAAGGATAGCTTTCTCGGAC |
| | ATCTCAATTACACGACTGAGCAGGTTGAGATGCACACA |
| | GTGACAGTACAGTTGCAGGAATCGTTTGCGGCAAACC |
| | ACTTGTACTGCATTAGGAGAGGAGACTTGCTCACACCG |
| | GAGGTGCGTACTTTCGGCCAACCTGACAGGTATGTGAT |
| | TCCACCACAGATCTTTCTCCCAAAAGTTCACAACTGCA |
| | AGAAGCCGATTCTTAAGAAAACTATGATGCAGCTCTTC |
| | TTGTATGTTAGGACAGTCAAGGTCGCAAAAAATTGTGA |
| | CATCTTTGCCAAAGTCAGACAATTAATTAAATCATCTG |
| | ACTTAGACAAATATTCTGCTGTGGAACTGGTTTACTTA |
| | GTAAGCTATATGGAGTTCCTTGCCGATTTACAAGCTAC |
| | CACCTGCTTCTCAGACACACTCTCTGGTGGCTTGCTAA |
| | CAAAGACCCTTGCACCGGTGAGGGCTTGGATACAAGA |
| | AAAGAAGATGCAGCTGTTTGGTCTTGAGGACTACGCG |
| | AAGTTAGTCAAAGCAGTTGATTTCCACCCGGTGGATTT |
| | TTCTTTCAAAGTGGAAACTTGGGACTTCAGATTCCACC |
| | CCTTGCAAGCGTGGAAAGCCTTCCGACCAAGGGAAGT |
| | GTCGGATGTAGAGGAAATGGAAAGTTTGTTCTCAGATG |
| | GGGACCTGCTTGATTGCTTCACAAGAATGCCAGCTTAT |
| | GCAGTAAACGCAGAGGAAGATTTAGCTACAATCAGGA |
| | AAACGCCCGAGATGGATGTCGGTCAAGAAGTTAAAGA |
| | GCCTGCAGGAGACAGAAATCAATACTCAAACCCTGCA |
| | GAAACTTTCCTCAACAAGCTCCACAGGAAACACAGTA |
| | GGGAGGTGAAACACCAGGCCGTAAAGAAAGCTAAACG |
| | CCTAGCTGAAATCCAGGAGTCCATGAGAGCTGAAGGT |
| | GAGGCCGAACCAAATGAGACGAGCGGGGGCATGGGG |
| | GCAATACCCAGCAACGCCGAACTTCCCGGCACGAGTG |
| | ATGCCAGACAAGAACTCACACTCCCAACCACTAAACCT |
| | GTTCCTGCAAGGTGGGAAGATGCTTCATTCACAGATTC |
| | TAGTGTGGAAGAGGAGCAGGTAAGACTCCTTGGAGAA |
| | GAAGCAGTGAAAACAGCGACGCAGCAAGTCATCGAAG |
| | GACTCCCTTGGAAACACTGGATTCCTCAACTAAATGCT |
| | GTTGGATTCAAGGCGCTGTTAATCCAGAGGGATAGGA |
| | GTGGAACGATGATCATGCCCATCACAGAAATGGTCTCC |
| | GGGTTGGAAAAAGAGGACTTCCCGGAAGGAACTCCAA |
| | AAGAGTTGGCACGAGAATTACTCGTTATGAACAGAAG |
| | CCCTGCCACCATCCCTTTGGACCTGCTTAGAGC |
| PVX Genome Segment 2 | TTGGCACGAGAATTACTCGTTATGAACAGAAGCCCTGC |
| cloned into pCMV-T7 | CACCATCCCTTTGGACCTGCTTAGAGCCAGAGACTACG |
| 2131 nt-3813 nt of the | GCAGTGATGTAAAGAACAAGAGAATTGGTGCCATCAC |
| PVX Genome | AAAGACACAGGCAACGAGTTGGGGCGAGTACTTAACA |
| | GGAAAGATAGAAAGCCTGACTGAGAGGAAAGTTGCGA |
| | CTTGTGTCATTCATGGAGCTGGAGGCTCTGGGAAAAGT |
| | CATGCCATCCAGAAGGCACTGAGAGAAATTGGCAAGG |
| | GCTCGGACATCACTGTAGTCCTGCCGACCAATGAACTG |
| | CGGCTAGATTGGAGTAAGAAGGTGCCTAACACTGAAC |
| | CCTATATGTTCAAGACCTACGAAAAGGCGTTAATTGGG |
| | GGAACAGGCAGCATAGTCATCTTTGACGATTACTCAAA |
| | ACTTCCTCCCGGTTACATAGAAGCCTTAGTCTGTTTCTA |
| | CTCCAAAATCAAGCTAATCATTCTAACAGGAGATAGCA |
| | GACAGAGCGTCTACCATGAAACTGCTGAGGACGCCTC |
| | CATCAGGCATTTGGGGCCAGCGACAGAGTACTTCTCAA |
| | AATACTGCCGATACTATCTCAATGCTACACACCGCAAC |
| | AAGAAAGACCTTGCGAACATGCTTGGTGTCTACAGTGA |
| | GAGAACGGGAGTCACTGAAATCAGCATGAGCGCCGAG |
| | TTTTTAGAAGGAATCCCAACTTTAGTACCCTCGGATGA |
| | GAAGAGAAAGCTGTACATGGGCACCGGGAGGAATGAC |
| | ACATTCACATACGCTGGATGCCAGGGGCTAACTAAGCC |
| | GAAAGTACAAATAGTGTTGGACCACAACACCCAAGTG |
| | TGTAGTGCGAATGTGATGTACACGGCACTTTCTAGAGC |
| | CACCGATAGGATTCACTTCGTGAACACAAGTGCAAACT |
| | CCTCGGCCTTCTGGGAAAAGTTGGACAGCACCCCTTAC |
| | CTCAAGACTTTCCTATCAGTGGTGAGAGAACAAGCACT |
| | CAGGGAGTATGAGCCGGCAGAGGCAGAGCCAATTCAA |
| | GAGCCTGAGCCCCAGACACACATGTGTGTCGAGAATG |
| | AGGAGTCCGTGCTAGAAGAGTACAAAGAGGAACTCTT |
| | GGAAAAGTTTGACAGAGAGATACACTCTGAATCCCAT |
| | GGTCATTCAAACTGTGTCCAAACTGAAGACACAACCAT |
| | TCAGTTGTTTTCGCATCAACAAGCAAAAGATGAGACCC |
| | TCCTCTGGGCGACCATAGATGCGCGGCTCAAGACTAGC |
| | AATCAAGAGGCAAACTTCCGAGAATTCCTGAGCAAGA |
| | AGGACATTGGGGACGTTCTGTTTTTAAACTACCAAAAA |
| | GCTATGGGTTTGCCCAAAGAGCGTATTCCTTTTTCCCA |
| | AGAGGTCTGGGAAGCTTGTGCCCACGAAGTACAAAGC |
| | AAGTACCTCAGTAAGTCAAAGTGCAACTTGATCAATGG |
| | GACTGTGAGACAGAGCCCAGACTTCGATGAAAACAAG |
| | ATTATGGTATTCCTCAAGTCGCAGTGGGTCACAAAGGT |
| | GGAAAAACTAGGTCTACCCAAGATTAAGCCAGGTCAA |
| | ACCATAGCAGCCTTTTACCAGCAGACTGTGATGCTTTT |
| | TGGAACTATGGCTAGGTACATGCGATGGTTCAGACAG |
| | GCTTTCCAGCCAAAAGAAGTCTTCATAAACTGTGAGAC |
| | TACGCCAGAAGACATGTCTGTATGGGCCTTGAACAACT |
| | GGAATTTCAGCAGACCTAGCTTAGCTAATGACTAC |
| PVX Genome Segment 3 | TGTATGGGCCTTGAACAACTGGAATTTCAGCAGACCTA |
| cloned into pCMV-T7 | GCTTAGCTAATGACTACACAGCTTTCGACCAGTCTCAG |
| 3813 nt-5311 nt | GATGGAGCTATGCTGCAATTTGAGGTGCTCAAAGCCAA |
| of the | GCACCACTGCATACCAGAGGAAATCATCCAAGCATAC |
| PVX Genome | ATAGACATTAAGACCAATGCACAGATTTTCCTAGGCAC |
| | ATTATCGATTATGCGCCTGACTGGTGAGGGTCCCACTT |
| | TTGATGCAAACACTGAGTGCAACATAGCTTACACCCAC |
| | ACAAAGTTTGACATCCCAGCCGGAACTGCTCAAGTTTA |
| | TGCAGGAGACGACTCCGCACTGGATTGCGTTCCAGAA |
| | GTGAAGCATAGTTTCCACAGGCTTGAAGACAAATTACT |
| | CCTCAAGTCAAAGCCTGTAATCACGCAGCAAAAGAAA |
| | GGCAGTTGGCCTGAGTTTTGTGGTTGGCTGATCACACC |
| | AAAAGGGGTAATGAAAGACCCAATTAAGCTCCATGTT |
| | AGCTTAAAATTGGCCGAAGCTAAGGGTGAACTCAAGA |
| | AATGTCAAGATTCCTATGAAATTGATCTGAGTTATGCC |
| | TATGACCACAAGGACTCTCTGCATGACTTGTTCGATGA |
| | GAAACAGTGTCAGGCACATACACTCACTTGCAGGACA |
| | CTAATCAAGTCAGGGAGAGGCACTGTCTCACTTCCCCG |
| | CCTCAGAAACTTTCTTTAACCGTTAATTTACCTTATAGA |
| | TTTGAATAAGATGGATATTCTCATCAGTAGTTTGAAAA |
| | GTTTAGGTTATTCTAGGACTTCTAAATCTTTAGATTCAG |
| | GACCTTTGGTAGTACATGCAGTAGCCGGAGCAGGTAA |
| | GTCCACAGCCCTAAGGAAGTTGATCCTCAGACACCCAA |
| | CATTCACCGTGCATACACTCGGTGTCCCTGACAAGGTG |
| | AGTATCAGAACTAGAGGCATACAGAAGCCAGGACCTA |
| | TTCCTGAGGGCAATTTCGCAATCCTCGATGAGTATACT |
| | TTGGACAACACCACAAGGAACTCATACCAGGCACTTTT |
| | TGCTGACCCTTATCAGGCACCTGAGTTTAGCCTAGAGC |
| | CCCACTTCTACTTGGAAACATCATTTCGAGTTCCGAGG |
| | AAAGTGGCAGATTTGATAGCTGGCTGTGGCTTCGATTT |
| | CGAGACTAACTCACAGGAAGAAGGGCATTTAGAGATC |
| | ACTGGCATATTCAAAGGGCCCCTACTTGGAAAGGTGAT |
| | AGCCATTGATGAGGAGTCTGAGACAACACTGTCCAGG |
| | CATGGTGTTGAGTTTGTTAAGCCCTGCCAAGTGACTGG |
| | ACTTGAGTTGAAAGTAGTCACTATTGTGTCTGCCGCAC |
| | CAATAGAGGAAATTGGCCAGTCCACAGCTTTCTACAAC |
| | GCTATCACCAGGTCAAAGGGATTGACATATGTCCGCGC |
| | AGGGACATAGACTGACCGCTCCGGTCAATTCTGAAAA |
| | AGTGTACATAGTATTAGGTCTATCATTTGCTTTAGTTTC |
| | AATTACTTTCCTGCTTTCTAGAAATAGTTTGCCCCACGT |
| | CGGTGACAACATTCACAGCTTGCCACACGGAGGAGCTT |
| | AC |
| PVX Genome Segment 4 | GACCGCTCCGGTCAATTCTGAAAAAGTGTACATAGTAT |
| cloned into pCMV-T7 | TAGGTCTATCATTTGCTTTAGTTTCAATTACTTTCCTGC |
| 5311 nt-6435 nt | TTTCTAGAAATAGTTTGCCCCACGTCGGTGACAACATT |
| of the | CACAGCTTGCCACACGGAGGAGCTTACAGAGACGGCA |
| PVX Genome | CCAAAGCAATCTTGTACAACTCCCCAAATCTAGGGTCA |
| | CGAGTGAGTCTACACAACGGAAAGAACGCAGCATTTG |
| | CTGCCGTTTTGCTACTGACTTTGCTGATCTATGGAAGTA |
| | AATACATATCTCAACGCAATCATACTTGTGCTTGTGGT |
| | AACAATCATAGCAGTCATTAGTACTTCCTTAGTGAGGA |
| | CTGAACCTTGTGTCATCAAGATTACTGGGGAATCAATC |
| | ACAGTGTTGGCTTGCAAATTAGATGCAGAAACTATAAA |
| | AGCCATTGCCGATCTCAAGCCACTCTCCGTTGAACGGT |
| | TAAGTTTCCATTGATACTCGAAAGATGTCAGCACCAGC |
| | TAGCACAACACAGGCCACAGGGTCAACTACCTCAACT |
| | ACCACGAAAACTGCAGGCGCAACTCCTGCCACAGCTTC |
| | AGGCCTGTTCACCATCCCGGATGGGGATTTCTTTAGTA |
| | CAGCTCGTGCCATAGTAGCCAGCAATGCTGTCGCAACA |
| | AATGAGGACCTCAGCAAGATTGAGGCTATTTGGAAGG |
| | ACATGAAGGTGCCCACAGACACTATGGCACAGGCTGC |
| | TTGGGACTTAGTCAGACACTGTGCTGATGTGGGATCAT |
| | CTGCTCAAACAGAAATGATAGATACAGGTCCTTATTCC |
| | AACGGCATCAGCAGAGCTAGACTGGCAGCAGCAATCA |
| | AAGAGGTGTGCACACTTAGGCAATTTTGCATGAAGTAT |
| | GCTCCAGTGGTATGGAACTGGATGTTAACTAACAACAG |
| | TCCACCTGCTAACTGGCAAGCACAAGGTTTCAAGCCTG |
| | AGCACAAATTCGCTGCATTCGACTTCTTCAATGGAGTC |
| | ACCAACCCAGCTGCCATCATGCCCAAAGAGGGGCTCA |
| | TCCGGCCACCGTCTGAAGCTGAAATGAATGCTGCCCAA |
| | ACTGCTGCTTTTGTGAAGATTACAAAGGCCAGGGCACA |
| | ATCCAACGACTTTGCCAGCCTAGATGCAGCTGTCACTC |
| | GAGGTCGTATCACTGGAACAACAACCGCTGAGGCTGTT |
| | GTCACTCTACCACCACCATAACTACGTCTACATAACCG |
| | ACGCCTACCCCAGTTTCATAGTATTTTCTGGTTTGATTG |
| | TATGAATAATATAAATTCTAGAGCTGGAGCCTCGGTAG |
| | CCGTTCCTCCTGCCCGCTGGGCCTCCCAACGGGCCCTC |
| | CTCCCCTCCTTGCACCGGCCCTTCCTGGTCTTTGCCGCG |
| | GCTGTGCCTTCTAGTTGCCAGCCATCTGTTGTTTGCCCC |
| | TCCCCCGTGCC |
| PVX CP into pHGWA- | AGATCCAGATCTCGATCCCGCGAAATTAATACGACTCA |
| 9xHis | CTATAGGGGAATTGTGAGCGGATAACAATTCCCCTCTA |
| | GAAATAATTTTGTTTAACTTTAAGAAGGAGATATACCA |
| | TGGGCAGCAGCCATCATCATCATCATCATCATCATCAC |
| | GGTGAGAATCTTTATTTTCAGGGCATGTCAGCACCAGC |
| | TAGCACAACACAGACCATAGGGTCAACTACCTCAACT |
| | ACCACAAAAACTGCAGGCGCAACTCCTGCCACAGCTTC |
| | AGGACTGTTCACCATCCCGGATGGGGATTTCTTTAACA |
| | CAGCCCGTGCCATAGTAGCCAGCAATGCCGTTGCAACG |
| | AATGAGGACCTCAGAAAGATTGAGGCTATTTGGAAAG |
| | ACATGAAGGTACCCACAGACACTATGGCACAGGCTGC |
| | TTGGGACTTGGTCAGACACTGTGCTGATGTGGGCTCAT |
| | CTGCCCAAACAGAGATGATAGATACAGGTCCTTATTCC |
| | AATGGCATCAGCAGAGCTAGACTGGCAGCAGCGATTA |
| | AAGAGGTGTGCACACTTAGACAATTCTGCATGAAGTAT |
| | GCCCCAGTGGTATGGAACTGGATGTTGACTAACAACA |
| | GTCCACCTGCTAACTGGCAAGCACAAGGTTTCAAGCCT |
| | GAGCACAAATTCGCTGCATTCGACTTCTTCAACGGAGT |
| | CACCAACCCAGCTGCCATCATGCCCAAAGAGGGACTC |
| | ATCCGGCCACCATCTGAAGCAGAAATGAATGCTGCCC |
| | AAACTGCTGCCTTTGTGAAAATTACGAAGGCCAGGGC |
| | ACAATCCAACGACTTTGCCAGCCTAGATGCGGCTGTCA |
| | CTCGAGGTCGTATCACTGGAACAACAACCGCTGAGGCT |
| | GTTGTCACTCTACCACCACCATAAAAGGGTGGGCGCGC |
| | CGACCCAGCTTTCTTGTACAAAGTGGTGATGTACCTCG |
| | AGCACCACCACCACCACCAC |

TABLE 4
Exemplary Plasmid

| | | | | | full length | length | percent | | |
|---|---|---|---|---|---|---|---|---|---|
| | start | end | | percent | of feature | of found | match | | |
| sseqid | location | location | strand | identity | in db | feature | length | fragment | database |
| EGFP | 2997 | 3717 | 1 | 100 | 720 | 720 | 100 | FALSE | snapgene |
| ori | 954 | 1543 | 1 | 100 | 589 | 589 | 100 | FALSE | snapgene |
| IRES_(2) | 2411 | 2984 | 1 | 100 | 573 | 573 | 100 | FALSE | snapgene |
| f1_ori | 5589 | 6018 | 1 | 100 | 429 | 429 | 100 | FALSE | snapgene |
| AmpR_promoter_(4) | 6071 | 6176 | 1 | 100 | 105 | 105 | 100 | FALSE | snapgene |
| bla (M) | 0 | 783 | 1 | 99.489 | 795 | 783 | 98.49056604 | FALSE | snapgene |
| WPRE_(3) | 3783 | 4310 | 1 | 97.232 | 589 | 542 | 92.02037351 | TRUE | snapgene |
| lac_promoter | 1866 | 1897 | 1 | 100 | 31 | 31 | 100 | FALSE | snapgene |
| CAP_binding_site | 1830 | 1852 | 1 | 100 | 22 | 22 | 100 | FALSE | snapgene |
| T7_promoter | 2185 | 2204 | 1 | 100 | 19 | 19 | 100 | FALSE | snapgene |
| lac_operator_(2) | 1904 | 1921 | 1 | 100 | 17 | 17 | 100 | FALSE | snapgene |
| lacZ_alpha | 5427 | 5585 | 1 | 100 | 174 | 158 | 90.8045977 | TRUE | snapgene |
| AAV2_ITR | 5283 | 5414 | 1 | 100 | 141 | 131 | 92.90780142 | TRUE | snapgene |
| AAV2_ITR | 1970 | 2101 | −1 | 100 | 141 | 131 | 92.90780142 | TRUE | snapgene |
| 1 | 2524 | 2981 | 1 | 100 | 462 | 457 | 98.91774892 | FALSE | Rfam |
| P11292 | 3783 | 4311 | 1 | 94.5 | 1668 | 528 | 31.65467626 | TRUE | swissprot |
| 2 | 993 | 1098 | −1 | 100 | 102 | 105 | 97.05882353 | FALSE | Rfam |
| 3 | 2207 | 2274 | 1 | 100 | 73 | 67 | 91.78082192 | TRUE | Rfam |
| T5_promoter | 1904 | 1923 | 1 | 100 | 45 | 19 | 42.22222222 | TRUE | snapgene |
| lacl | 1725 | 1818 | 1 | 100 | 1083 | 93 | 8.587257618 | TRUE | snapgene |
| AmpR_(2) | 6176 | 6253 | 1 | 100 | 861 | 77 | 8.943089431 | TRUE | snapgene |
| CMV_intron_(3) | 2185 | 2205 | 1 | 100 | 110 | 20 | 18.18181818 | TRUE | snapgene |
| Q02940 | 1816 | 1912 | 1 | 68.8 | 939 | 96 | 10.22364217 | TRUE | swissprot |
| PVX OAS | | | | | | | | | |

| | | | | SEQ |
|---|---|---|---|---|
| sseqid | Feature | Type | Description | ID NO. |
| EGFP | EGFP | CDS | the original enhanced GFP (Yang et al., | 5 |
| | | | 1996) mammalian codon-optimized | |
| ori | ori | rep_origin | high-copy-number ColE1/pMB1/pBR322/ | 6 |
| | | | pUC origin of replication | |
| IRES_(2) | IRES | misc_feature | internal ribosome entry site (IRES) | 7 |
| | | | of the encephalomyocarditis virus (EMCV) | |
| f1_ori | f1 ori | rep_origin | f1 bacteriophage origin of replication; | 8 |
| | | | arrow 9indicates direction of (+) | |
| | | | strand synthesis | |
| AmpR_promoter_(4) | AmpR promoter | promoter | bla | 9 |
| bla (M) | bla(M) | CDS | ÃŽÂ²-lactamase lacking the signal sequence; | 10 |
| | | | bla(M); allows cytosolic expression of | |
| | | | ÃŽÂ²-lactamase | |
| WPRE_(3) | WPRE | misc_feature | woodchuck hepatitis virus posttranscriptional | 11 |
| | | | regulatory element | |
| lac_promoter | lac promoter | promoter | promoter for the E. coli lac operon | 12 |
| CAP_binding_site | CAP binding | protein_bind | CAP binding activates transcription in the | 13 |
| | site | | presence of cAMP. E. coli catabolite | |
| | | | activator protein | |
| T7_promoter | T7 promoter | promoter | promoter for bacteriophage T7 RNA polymerase | 14 |
| lac_operator_(2) | lac operator | protein_bind | The lac repressor binds to the lac operator | 15 |
| | | | to inhibit transcription in E. coli. This | |
| | | | inhibition can be relieved by adding lactose | |
| | | | or isopropyl-ÃŽÂ²-D-thiogalactopyranoside | |
| | | | (IPTG). lac repressor encoded by lacl | |
| lacZ_alpha | lacZÃŽÂ± | CDS | LacZÃŽÂ± fragment of ÃŽÂ²-galactosidase; | 16 |
| | | | lacZ fragment | |
| AAV2_ITR | AAV2 ITR | repeat_region | inverted terminal repeat of adeno-associated | 17 |
| | | | virus serotype 2 inverted | |
| AAV2_ITR | AAV2 ITR | repeat_region | inverted terminal repeat of adeno-associated | 18 |
| | | | virus serotype 2 inverted | |
| 1 | IRES Picorna 2 | ncRNA | Accession: RF00210 - Aphthovirus internal | 19 |
| | | | ribosome entry site (IRES) | |
| P11292 | P | CDS | DPOL_WHV6 - Experimental evidence at transcript | 20 |
| | | | level: Swiss-Prot protein existence level 2. | |
| | | | Multifunctional enzyme that converts the viral | |
| | | | RNA genome into dsDNA in viral cytoplasmic capsids. | |
| | | | This enzyme displays a DNA polymerase activity | |
| | | | that can copy either DNA or RNA templates, and a | |
| | | | ribonuclease H (RNase H) activity that cleaves | |
| | | | the RNA strand of RNA-DNA heteroduplexes in a | |
| | | | partially processive 3′- to 5′-endonucleasic | |
| | | | mode. Neo-synthesized pregenomic RNA (pgRNA) | |
| | | | are encapsidated together with the P protein, | |
| | | | and reverse-transcribed inside the nucleocapsid. | |
| | | | Initiation of reverse-transcription occurs first | |
| | | | by binding the epsilon loop on the pgRNA genome, | |
| | | | and is initiated by protein priming, thereby the | |
| | | | 5′-end of (−)DDNA is covalently linked | |
| | | | to P protein. Partial (+)DNA is synthesized | |
| | | | from the (−)DNA template and generates | |
| | | | the relaxed circular DNA (RC-DNA) genome. | |
| | | | After budding and infection, the RC-DNA migrates | |
| | | | in the nucleus, and is converted into a | |
| | | | plasmid-like covalently closed circular DNA | |
| | | | (cccDNA). The activity of P protein does not seem | |
| | | | to be necessary for cccDNA generation, and is | |
| | | | presumably released from (+)DNA by host | |
| | | | nuclear DNA repair machinery (By similarity). | |
| | | | From Woodchuck hepatitis B virus (isolate w64/ | |
| | | | pWS23) (WHV). | |
| 2 | RNAI | ncRNA | Accession: RF00106 - RNAI | 21 |
| 3 | twister-P3 | ncRNA | Accession: RF03154 - type-P3 twister ribozyme | 22 |
| T5_promoter | T5 promoter | promoter | bacteriophage T5 promoter for E. coli RNA | 23 |
| | | | polymerase, with embedded lac operator | |
| lacl | lacl | CDS | lac repressor; lacl; The lac repressor binds to | 24 |
| | | | the lac operator to inhibit transcription in | |
| | | | E. coli. This inhibition can be relieved by adding | |
| | | | lactose or isopropyl-ÃŽÂ²-D-thiogalactopyranoside | |
| | | | (IPTG). | |
| AmpR_(2) | AmpR | CDS | ÃŽÂ²-lactamase; bla; confers resistance to | 25 |
| | | | ampicillin, carbenicillin, and related antibiotics | |
| CMV_intron_(3) | CMV intron | intron | modified intron A from human cytomegalovirus (CMV) | 26 |
| Q02940 | penA | CDS | PENA_BURM1 - Experimental evidence at transcript | 27 |
| | | | level: Swiss-Prot protein existence level 2. | |
| | | | Upon expression in E. coli enables the latter | |
| | | | to utilize penicillin as a carbon source. From | |
| | | | Burkholderia multivorans (strain ATCC 17616/249). | |
| PVX OAS | | | | |

SEQ ID NO: 5
ATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGG
GCGAGGGCGATGCCACCTACGGCAAGCTGACCCTGAAGTTCATCTGCACC
ACCGGCAAGCTGCCCGTGCCCTGGCCCACCCTCGTGACCACCCTGACCTA
CGGCGTGCAGTGCTTCAGCCGCTACCCCGACCACATGAAGCAGCACGACT
TCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGG
CGACACCCTGGTGAACCGCATCGAGCTGAAGGGCATCGACTTCAAGGAGG
ACGGCAACATCCTGGGGCACAAGCTGGAGTACAACTACAACAGCCACAAC
GTCTATATCATGGCCGACAAGCAGAAGAACGGCATCAAGGTGAACTTCAA
GATCCGCCACAACATCGAGGACGGCAGCGTGCAGCTCGCCGACCACTACC
AGCAGAACACCCCCATCGGCGACGGCCCCGTGCTGCTGCCCGACAACCAC
TACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCA
TGGACGAGCTGTACAAGTAA
SEQ ID NO: 6
TTGAGATCCTTTTTTTCTGCGCGTAATCTGCTGCTTGCAAACAAAAAAAC
CACCGCTACCAGCGGTGGTTTGTTTGCCGGATCAAGAGCTACCAACTCTT
TTTCCGAAGGTAACTGGCTTCAGCAGAGCGCAGATACCAAATACTGTCCT
TCTAGTGTAGCCGTAGTTAGGCCACCACTTCAAGAACTCTGTAGCACCGC
CTACATACCTCGCTCTGCTAATCCTGTTACCAGTGGCTGCTGCCAGTGGC
GATAAGTCGTGTCTTACCGGGTTGGACTCAAGACGATAGTTACCGGATAA
GGCGCAGCGGTCGGGCTGAACGGGGGGTTCGTGCACACAGCCCAGCTTGG
AGCGAACGACCTACACCGAACTGAGATACCTACAGCGTGAGCTATGAGAA
AGCGCCACGCTTCCCGAAGGGAGAAAGGCGGACAGGTATCCGGTAAGCGG
CAGGGTCGGAACAGGAGAGCGCACGAGGGAGCTTCCAGGGGGAAACGCCT
GGTATCTTTATAGTCCTGTCGGGTTTCGCCACCTCTGACTTGAGCGTCGA
TTTTTGTGATGCTCGTCAGGGGGGCGGAGCCTATGGAAA
SEQ ID NO: 7
CCCCTCTCCCTCCCCCCCCCCTAACGTTACTGGCCGAAGCCGCTTGGAAT
AAGGCCGGTGTGCGTTTGTCTATATGTTATTTTCCACCATATTGCCGTCT
TTTGGCAATGTGAGGGCCCGGAAACCTGGCCCTGTCTTCTTGACGAGCAT
TCCTAGGGGTCTTTCCCCTCTCGCCAAAGGAATGCAAGGTCTGTTGAATG
TCGTGAAGGAAGCAGTTCCTCTGGAAGCTTCTTGAAGACAAACAACGTCT
GTAGCGACCCTTTGCAGGCAGCGGAACCCCCCACCTGGCGACAGGTGCCT
CTGCGGCCAAAAGCCACGTGTATAAGATACACCTGCAAAGGCGGCACAAC
CCCAGTGCCACGTTGTGAGTTGGATAGTTGTGGAAAGAGTCAAATGGCTC
TCCTCAAGCGTATTCAACAAGGGGCTGAAGGATGCCCAGAAGGTACCCCA
TTGTATGGGATCTGATCTGGGGCCTCGGTGCACATGCTTTACATGTGTTT
AGTCGAGGTTAAAAAACGTCTAGGCCCCCCGAACCACGGGGACGTGGTTT
TCCTTTGAAAAACACGATGATAA
SEQ ID NO: 8
ACGCGCCCTGTAGCGGCGCATTAAGCGCGGCGGGTGTGGTGGTTACGCGC
AGCGTGACCGCTACACTTGCCAGCGCCCTAGCGCCCGCTCCTTTCGCTTT
CTTCCCTTCCTTTCTCGCCACGTTCGCCGGCTTTCCCCGTCAAGCTCTAA
ATCGGGGGCTCCCTTTAGGGTTCCGATTTAGTGCTTTACGGCACCTCGAC
CCCAAAAAACTTGATTAGGGTGATGGTTCACGTAGTGGGCCATCGCCCTG
ATAGACGGTTTTTCGCCCTTTGACGTTGGAGTCCACGTTCTTTAATAGTG
GACTCTTGTTCCAAACTGGAACAACACTCAACCCTATCTCGGTCTATTCT
TTTGATTTATAAGGGATTTTGCCGATTTCGGCCTATTGGTTAAAAAATGA
GCTGATTTAACAAAAATTTAACGCGAATT
SEQ ID NO: 9
CGCGGAACCCCTATTTGTTTATTTTTCTAAATACATTCAAATATGTATCC
GCTCATGAGACAATAACCCTGATAAATGCTTCAATAATATTGAAAAAGGA
AGAGT
SEQ ID NO: 10
AACGCTGGTGAAAGTAAAAGATGCTGAAGATCAGTTGGGTGCACGAGTGG
GTTACATCGAACTGGATCTCAATAGTGGTAAGATCCTTGAGAGTTTTCGC
CCCGAAGAACGTTTTCCAATGATGAGCACTTTTAAAGTTCTGCTATGTGG
CGCGGTATTATCCCGTATTGACGCCGGGCAAGAGCAACTCGGTCGCCGCA
TACACTATTCTCAGAATGACTTGGTTGAGTACTCACCAGTCACAGAAAAG
CATCTTACGGATGGCATGACAGTAAGAGAATTATGCAGTGCTGCCATAAC
CATGAGTGATAACACTGCGGCCAACTTACTTCTGACAACGATCGGAGGAC
CGAAGGAGCTAACCGCTTTTTTGCACAACATGGGGGATCATGTAACTCGC
CTTGATCGTTGGGAACCGGAGCTGAATGAAGCCATACCAAACGACGAGCG
TGACACCACGATGCCTGTAGTAATGGTAACAACGTTGCGCAAACTATTAA
CTGGCGAACTACTTACTCTAGCTTCCCGGCAACAATTAATAGACTGGATG
GAGGCGGATAAAGTTGCAGGACCACTTCTGCGCTCGGCCCTTCCGGCTGG
CTGGTTTATTGCTGATAAATCTGGAGCCGGTGAGCGTGGGTCTCGCGGTA
TCATTGCAGCACTGGGGCCAGATGGTAAGCCCTCCCGTATCGTAGTTATC
TACACGACGGGGAGTCAGGCAACTATGGATGAACGAAATAGACAGATCGC
TGAGATAGGTGCCTCACTGATTAAGCATTGGTA
SEQ ID NO: 11
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGG
GGCTCGGCTGTTGGGCACTGACAATTCCGTGGTGTTGTCGGGGAAATCAT
CGTCCTTTCCTTGGCTGCTCGCCTGTGTTGCCACCTGGATTCTGCGCGGG
ACGTCCTTCTGCTA--------------CAATCCAGCGGACCTTCCTTCC
CGCGGCCTGCTGCCGGCTCTGCGGCCTCTTCCGCGTCTTCG
SEQ ID NO: 12
TTTACACTTTATGCTTCCGGCTCGTATGTTG
SEQ ID NO: 13
TAATGTGAGTTAGCTCACTCAT
SEQ ID NO: 14
TAATACGACTCACTATAGG
SEQ ID NO: 15
TTGTGAGCGGATAACAA
SEQ ID NO: 16
ATTCACTGGCCGTCGTTTTACAACGTCGTGACTGGGAAAACCCTGGCGTT
ACCCAACTTAATCGCCTTGCAGCACATCCCCCTTTCGCCAGCTGGCGTAA
TAGCGAAGAGGCCCGCACCGATCGCCCTTCCCAACAGTTGCGCAGCCTGA
ATGGCGAA
SEQ ID NO: 17
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGC
SEQ ID NO: 18
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGC
SEQ ID NO: 19
GGCCCGGAAACCTGGCCCTGTCTTCTTGACGAGCATTCCTAGGGGTCTTT
CCCCTCTCGCCAAAGGAATGCAAGGTCTGTTGAATGTCGTGAAGGAAGCA
GTTCCTCTGGAAGCTTCTTGAAGACAAACAACGTCTGTAGCGACCCTTTG
CAGGCAGCGGAACCCCCCACCTGGCGACAGGTGCCTCTGCGGCCAAAAGC
CACGTGTATAAGATACACCTGCAAAGGCGGCACAACCCCAGTGCCACGTT
GTGAGTTGGATAGTTGTGGAAAGAGTCAAATGGCTCTCCTCAAGCGTATT
CAACAAGGGGCTGAAGGATGCCCAGAAGGTACCCCATTGTATGGGATCTG
ATCTGGGGCCTCGGTGCACATGCTTTACATGTGTTTAGTCGAGGTTAAAA
AACGTCTAGGCCCCCCGAACCACGGGGACGTGGTTTTCCTTTGAAAAACA
CGATGAT
SEQ ID NO: 20
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGG
GGCTCGGCTGTTGGGCACTGACAATTCCGTGGTGTTGTCGGGGAAATCAT
CGTCCTTTCCTTGGCTGCTCGCCTGTGTTGCCACCTGGATTCTGCGCGGG
ACGTCCTTCTGCTACAATCCAGCGGACCTTCCTTCCCGCGGCCTGCTGCC
GGCTCTGCGGCCTCTTCCGCGTCTTCGA
SEQ ID NO: 21
AGTATTTGGTATCTGCGCTCTGCTGAAGCCAGTTACCTTCGGAAAAAGAG
TTGGTAGCTCTTGATCCGGCAAACAAACCACCGCTGGTAGCGGTGGTTTT
TTTGT
SEQ ID NO: 22
TCAGTCGCCGGTCCCAAGCCCGGATAAAATGGGAGGGGGGGGAAACCGCC
TAACCATGCCGACTGA
SEQ ID NO: 23
TTGTGAGCGGATAACAATT
SEQ ID NO: 24
GCGCCCAATACGCAAACCGCCTCTCCCCGCGCGTTGGCCGATTCATTAAT
GCAGCTGGCACGACAGGTTTCCCGACTGGAAAGCGGGCAGTGA
SEQ ID NO: 25
ATGAGTATTCAACATTTCCGTGTCGCCCTTATTCCCTTTTTTGCGGCATT
TTGCCTTCCTGTTTTTGCTCACCCAGA
SEQ ID NO: 26
TAATACGACTCACTATAGGG
SEQ ID NO: 27
GAGCGCAACGCAATTAATGTGAGTTAGCTCACTCATTAGGCACCCCAGGC
TTTACACTTTATGCTTCCGGCTCGTATGTTGTGTGGAATTGTGAGC
Plasmid sequences
pCMV-T7-SL1-EGFP-
Sequence:
(SEQ ID NO: 28)
ATATGCCAAGTACGCCCCCTATTGACGTCAATGACGGTAAATGGCCCGCC
TGGCATTATGCCCAGTACATGACCTTATGGGACTTTCCTACTTGGCAGTA
CATCTACGTATTAGTCATCGCTATTACCATGGTGATGCGGTTTTGGCAGT
ACATCAATGGGCGTGGATAGCGGTTTGACTCACGGGGATTTCCAAGTCTC
CACCCCATTGACGTCAATGGGAGTTTGTTTTGGCACCAAAATCAACGGGA
CTTTCCAAAATGTCGTAACAACTCCGCCCCATTGACGCAAATGGGCGGTA
GGCGTGTACGGTGGGAGGTCTATATAAGCAGAGCTGGTTTAGTGAACCGT
CAGATCCGCTAGAGATCCGCGGCCGCTAATACGACTCACTATAGGGAGAG
CCTTGTTACACACCCGCTTGAAAAAGCAAGTCTGACAAAAGGCCAAAGTG
CGCGAGGGCCACCATGGTGAGCAAGGGCGAGGAGCTGTTCACCGGGGTGG
TGCCCATCCTGGTCGAGCTGGACGGCGACGTAAACGGCCACAAGTTCAGC
GTGTCCGGCGAGGGCGAGGGCGATGCCACCTACGGCAAGCTGACCCTGAA
GTTCATCTGCACCACCGGCAAGCTGCCCGTGCCCTGGCCCACCCTCGTGA
CCACCCTGACCTACGGCGTGCAGTGCTTCAGCCGCTACCCCGACCACATG
AAGCAGCACGACTTCTTCAAGTCCGCCATGCCCGAAGGCTACGTCCAGGA
GCGCACCATCTTCTTCAAGGACGACGGCAACTACAAGACCCGCGCCGAGG
TGAAGTTCGAGGGCGACACCCTGGTGAACCGCATCGAGCTGAAGGGCATC
GACTTCAAGGAGGACGGCAACATCCTGGGGCACAAGCTGGAGTACAACTA
CAACAGCCACAACGTCTATATCATGGCCGACAAGCAGAAGAACGGCATCA
AGGTGAACTTCAAGATCCGCCACAACATCGAGGACGGCAGCGTGCAGCTC
GCCGACCACTACCAGCAGAACACCCCCATCGGCGACGGCCCCGTGCTGCT
GCCCGACAACCACTACCTGAGCACCCAGTCCGCCCTGAGCAAAGACCCCA
ACGAGAAGCGCGATCACATGGTCCTGCTGGAGTTCGTGACCGCCGCCGGG
ATCACTCTCGGCATGGACGAGCTGTACAAGTGACCGGTCATCATCACCAT
CACCATTGAGTTTAAACCCGCTGATCAGCCTCGACTGTGCCTTCTAGTTG
CCAGCCATCTGTTGTTTGCCCCTCCCCCGTGCCTTCCTTGACCCTGGAAG
GTGCCACTCCCACTGTCCTTTCCTAATAAAATGAGGAAATTGCATCGCAT
TGTCTGAGTAGGTGTCATTCTATTCTGGGGGGTGGGGGGGGCAGGACAGC
AAGGGGGAGGATTGGGAAGACAATAGCAGGCATGCTGGGGATGCGGTGGG
CTCTATGGCTTCTGAGGCGGAAAGAACCAGCTGGGGCTCGATACCGTCGA
CCTCTAGCTAGAGCTTGGCGTAATCATGGTCATAGCTGTTTCCTGTGTGA
AATTGTTATCCGCTCACAATTCCACACAACATACGAGCCGGAAGCATAAA
GTGTAAAGCCTAGGGTGCCTAATGAGTGAGCTAACTCACATTAATTGCGT
TGCGCTCACTGCCCGCTTTCCAGTCGGGAAACCTGTCGTGCCAGCTGCAT
TAATGAATCGGCCAACGCGCGGGGAGAGGCGGTTTGCGTATTGGGCGCTC
TTCCGCTTCCTCGCTCACTGACTCGCTGCGCTCGGTCGTTCGGCTGCGGC
GAGCGGTATCAGCTCACTCAAAGGCGGTAATACGGTTATCCACAGAATCA
GGGGATAACGCAGGAAAGAACATGTGAGCAAAAGGCCAGCAAAAGGCCAG
GAACCGTAAAAAGGCCGCGTTGCTGGCGTTTTTCCATAGGCTCCGCCCCC
CTGACGAGCATCACAAAAATCGACGCTCAAGTCAGAGGTGGCGAAACCCG
ACAGGACTATAAAGATACCAGGCGTTTCCCCCTGGAAGCTCCCTCGTGCG
CTCTCCTGTTCCGACCCTGCCGCTTACCGGATACCTGTCCGCCTTTCTCC
CTTCGGGAAGCGTGGCGCTTTCTCAATGCTCACGCTGTAGGTATCTCAGT
TCGGTGTAGGTCGTTCGCTCCAAGCTGGGCTGTGTGCACGAACCCCCCGT
TCAGCCCGACCGCTGCGCCTTATCCGGTAACTATCGTCTTGAGTCCAACC
CGGTAAGACACGACTTATCGCCACTGGCAGCAGCCACTGGTAACAGGATT
AGCAGAGCGAGGTATGTAGGCGGTGCTACAGAGTTCTTGAAGTGGTGGCC
TAACTACGGCTACACTAGAAGGACAGTATTTGGTATCTGCGCTCTGCTGA
AGCCAGTTACCTTCGGAAAAAGAGTTGGTAGCTCTTGATCCGGCAAACAA
ACCACCGCTGGTAGCGGTGGTTTTTTTGTTTGCAAGCAGCAGATTACGCG
CAGAAAAAAAGGATCTCAAGAAGATCCTTTGATCTTTTCTACGGGGTCTG
ACGCTCAGTGGAACGAAAACTCACGTTAAGGGATTTTGGTCATGAGATTA
TCAAAAAGGATCTTCACCTAGATCCTTTTAAATTAAAAATGAAGTTTTAA
ATCAATCTAAAGTATATATGAGTAAACTTGGTCTGACAGTTACCAATGCT
TAATCAGTGAGGCACCTATCTCAGCGATCTGTCTATTTCGTTCATCCATA
GTTGCCTGACTCCCCGTCGTGTAGATAACTACGATACGGGAGGGCTTACC
ATCTGGCCCCAGTGCTGCAATGATACCGCGAGACCCACGCTCACCGGCTC
CAGATTTATCAGCAATAAACCAGCCAGCCGGAAGGGCCGAGCGCAGAAGT
GGTCCTGCAACTTTATCCGCCTCCATCCAGTCTATTAATTGTTGCCGGGA
AGCTAGAGTAAGTAGTTCGCCAGTTAATAGTTTGCGCAACGTTGTTGCCA
TTGCTACAGGCATCGTGGTGTCACGCTCGTCGTTTGGTATGGCTTCATTC
AGCTCCGGTTCCCAACGATCAAGGCGAGTTACATGATCCCCCATGTTGTG
CAAAAAAGCGGTTAGCTCCTTCGGTCCTCCGATCGTTGTCAGAAGTAAGT
TGGCCGCAGTGTTATCACTCATGGTTATGGCAGCACTGCATAATTCTCTT
ACTGTCATGCCATCCGTAAGATGCTTTTCTGTGACTGGTGAGTACTCAAC
CAAGTCATTCTGAGAATAGTGTATGCGGCGACCGAGTTGCTCTTGCCCGG
CGTCAATACGGGATAATACCGCGCCACATAGCAGAACTTTAAAAGTGCTC
ATCATTGGAAAACGTTCTTCGGGGCGAAAACTCTCAAGGATCTTACCGCT
GTTGAGATCCAGTTCGATGTAACCCACTCGTGCACCCAACTGATCTTCAG
CATCTTTTACTTTCACCAGCGTTTCTGGGTGAGCAAAAACAGGAAGGCAA
AATGCCGCAAAAAAGGGAATAAGGGCGACACGGAAATGTTGAATACTCAT
ACTCTTCCTTTTTCAATATTATTGAAGCATTTATCAGGGTTATTGTCTCA
TGAGCGGATACATATTTGAATGTATTTAGAAAAATAAACAAATAGGGGTT
CCGCGCACATTTCCCCGAAAAGTGCCACCTGACGTCGACGGATCGGGAGA
TCGATCTCCCGATCCCCTAGGGTCGACTCTCAGTACAATCTGCTCTGATG
CCGCATAGTTAAGCCAGTATCTGCTCCCTGCTTGTGTGTTGGAGGTCGCT
GAGTAGTGCGCGAGCAAAATTTAAGCTACAACAAGGCAAGGCTTGACCGA
CAATTGCATGAAGAATCTGCTTAGGGTTAGGCGTTTTGCGCTGCTTCGCG
ATGTACGGGCCAGATATACGCGTTGACATTGATTATTGACTAGTTATTAA
TAGTAATCAATTACGGGGTCATTAGTTCATAGCCCATATATGGAGTTCCG
CGTTACATAACTTACGGTAAATGGCCCGCCTGGCTGACCGCCCAACGACC
CCCGCCCATTGACGTCAATAATGACGTATGTTCCCATAGTAACGCCAATA
GGGACTTTCCATTGACGTCAATGGGTGGACTATTTACGGTAAACTGCCCA
CTTGGCAGTACATCAAGTGTATC
Circ-EMCV-GFP-SL1-
Sequence:
(SEQ ID NO: 29)
AACGCTGGTGAAAGTAAAAGATGCTGAAGATCAGTTGGGTGCACGAGTGG
GTTACATCGAACTGGATCTCAATAGTGGTAAGATCCTTGAGAGTTTTCGC
CCCGAAGAACGTTTTCCAATGATGAGCACTTTTAAAGTTCTGCTATGTGG
CGCGGTATTATCCCGTATTGACGCCGGGCAAGAGCAACTCGGTCGCCGCA
TACACTATTCTCAGAATGACTTGGTTGAGTACTCACCAGTCACAGAAAAG
CATCTTACGGATGGCATGACAGTAAGAGAATTATGCAGTGCTGCCATAAC
CATGAGTGATAACACTGCGGCCAACTTACTTCTGACAACGATCGGAGGAC
CGAAGGAGCTAACCGCTTTTTTGCACAACATGGGGGATCATGTAACTCGC
CTTGATCGTTGGGAACCGGAGCTGAATGAAGCCATACCAAACGACGAGCG
TGACACCACGATGCCTGTAGTAATGGTAACAACGTTGCGCAAACTATTAA
CTGGCGAACTACTTACTCTAGCTTCCCGGCAACAATTAATAGACTGGATG
GAGGCGGATAAAGTTGCAGGACCACTTCTGCGCTCGGCCCTTCCGGCTGG
CTGGTTTATTGCTGATAAATCTGGAGCCGGTGAGCGTGGGTCTCGCGGTA
TCATTGCAGCACTGGGGCCAGATGGTAAGCCCTCCCGTATCGTAGTTATC
TACACGACGGGGAGTCAGGCAACTATGGATGAACGAAATAGACAGATCGC
TGAGATAGGTGCCTCACTGATTAAGCATTGGTAACTGTCAGACCAAGTTT
ACTCATATATACTTTAGATTGATTTAAAACTTCATTTTTAATTTAAAAGG
ATCTAGGTGAAGATCCTTTTTGATAATCTCATGACCAAAATCCCTTAACG
TGAGTTTTCGTTCCACTGAGCGTCAGACCCCGTAGAAAAGATCAAAGGAT
CTTCTTGAGATCCTTTTTTTCTGCGCGTAATCTGCTGCTTGCAAACAAAA
AAACCACCGCTACCAGCGGTGGTTTGTTTGCCGGATCAAGAGCTACCAAC
TCTTTTTCCGAAGGTAACTGGCTTCAGCAGAGCGCAGATACCAAATACTG
TCCTTCTAGTGTAGCCGTAGTTAGGCCACCACTTCAAGAACTCTGTAGCA
CCGCCTACATACCTCGCTCTGCTAATCCTGTTACCAGTGGCTGCTGCCAG
TGGCGATAAGTCGTGTCTTACCGGGTTGGACTCAAGACGATAGTTACCGG
ATAAGGCGCAGCGGTCGGGCTGAACGGGGGGTTCGTGCACACAGCCCAGC
TTGGAGCGAACGACCTACACCGAACTGAGATACCTACAGCGTGAGCTATG
AGAAAGCGCCACGCTTCCCGAAGGGAGAAAGGCGGACAGGTATCCGGTAA
GCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCG
TCGATTTTTGTGATGCTCGTCAGGGGGGCGGAGCCTATGGAAAAACGCCA
GCAACGCGGCCTTTTTACGGTTCCTGGCCTTTTGCTGCGGTTTTGCTCAC
ATGTTCTTTCCTGCGTTATCCCCTGATTCTGTGGATAACCGTATTACCGC
CTTTGAGTGAGCTGATACCGCTCGCCGCAGCCGAACGACCGAGCGCAGCG
AGTCAGTGAGCGAGGAAGCGGAAGAGCGCCCAATACGCAAACCGCCTCTC
CCCGCGCGTTGGCCGATTCATTAATGCAGCTGGCACGACAGGTTTCCCGA
CTGGAAAGCGGGCAGTGAGCGCAACGCAATTAATGTGAGTTAGCTCACTC
ATTAGGCACCCCAGGCTTTACACTTTATGCTTCCGGCTCGTATGTTGTGT
GGAATTGTGAGCGGATAACAATTTCACACAGGAAACAGCTATGACCATGA
TTACGCCAGATTTAATTAAGGCTGCGCGCTCGCTCGCTCACTGAGGCCGC
CCGGGCAAAGCCCGGGCGTCGGGCGACCTTTGGTCGCCCGGCCTCAGTGA
GCGAGCGAGCGCGCAGAGAGGGAGTGGCCAACTCCATCACTAGGGGTTCC
TTGTAGTTAATGATTAACCCGCCATGCTACTTATCTACGTAGCCATGCTC
TAGGAAGATCGGAATTCGCCCTTAAGCTAGCATGCTAATACGACTCACTA
TAGGGCCATCAGTCGCCGGTCCCAAGCCCGGATAAAATGGGAGGGGGCGG
GAAACCGCCTAACCATGCCGACTGATGGCAGCTATTATCGAGCGAACGCC
TTATGCGATGAAAGTCGCACGTAGGGTGTAGACCAAGCGAAATCCTATGC
ATTTAGGATAGTGAGGTATAGCAAAGGAGAAGTCGACGGGCCCGCGGAAT
TCCGCCCCCCCCCCCTCTCCCTCCCCCCCCCCTAACGTTACTGGCCGAAG
CCGCTTGGAATAAGGCCGGTGTGCGTTTGTCTATATGTTATTTTCCACCA
TATTGCCGTCTTTTGGCAATGTGAGGGCCCGGAAACCTGGCCCTGTCTTC
TTGACGAGCATTCCTAGGGGTCTTTCCCCTCTCGCCAAAGGAATGCAAGG
TCTGTTGAATGTCGTGAAGGAAGCAGTTCCTCTGGAAGCTTCTTGAAGAC
AAACAACGTCTGTAGCGACCCTTTGCAGGCAGCGGAACCCCCCACCTGGC
GACAGGTGCCTCTGCGGCCAAAAGCCACGTGTATAAGATACACCTGCAAA
GGCGGCACAACCCCAGTGCCACGTTGTGAGTTGGATAGTTGTGGAAAGAG
TCAAATGGCTCTCCTCAAGCGTATTCAACAAGGGGCTGAAGGATGCCCAG
AAGGTACCCCATTGTATGGGATCTGATCTGGGGCCTCGGTGCACATGCTT
TACATGTGTTTAGTCGAGGTTAAAAAACGTCTAGGCCCCCCGAACCACGG
GGACGTGGTTTTCCTTTGAAAAACACGATGATAATATGGCCACAACCATG
GTGAGCAAGGGCGAGGAGCTGTTCACCGGGGTGGTGCCCATCCTGGTCGA
GCTGGACGGCGACGTAAACGGCCACAAGTTCAGCGTGTCCGGCGAGGGCG
AGGGCGATGCCACCTACGGCAAGCTGACCCTGAAGTTCATCTGCACCACC
GGCAAGCTGCCCGTGCCCTGGCCCACCCTCGTGACCACCCTGACCTACGG
CGTGCAGTGCTTCAGCCGCTACCCCGACCACATGAAGCAGCACGACTTCT
TCAAGTCCGCCATGCCCGAAGGCTACGTCCAGGAGCGCACCATCTTCTTC
AAGGACGACGGCAACTACAAGACCCGCGCCGAGGTGAAGTTCGAGGGCGA
CACCCTGGTGAACCGCATCGAGCTGAAGGGCATCGACTTCAAGGAGGACG
GCAACATCCTGGGGCACAAGCTGGAGTACAACTACAACAGCCACAACGTC
TATATCATGGCCGACAAGCAGAAGAACGGCATCAAGGTGAACTTCAAGAT
CCGCCACAACATCGAGGACGGCAGCGTGCAGCTCGCCGACCACTACCAGC
AGAACACCCCCATCGGCGACGGCCCCGTGCTGCTGCCCGACAACCACTAC
CTGAGCACCCAGTCCGCCCTGAGCAAAGACCCCAACGAGAAGCGCGATCA
CATGGTCCTGCTGGAGTTCGTGACCGCCGCCGGGATCACTCTCGGCATGG
ACGAGCTGTACAAGTAATTGTTACACACCCGCTTGAAAAAGCAAGTCTGA
CAAAAGGCCAAAGTGCGCGAGGGCCACGGATCCAATCAACCTCTGGATTA
CAAAATTTGTGAAAGATTGACTGGTATTCTTAACTATGTTGCTCCTTTTA
CGCTATGTGGATACGCTGCTTTAATGCCTTTGTATCATGCTATTGCTTCC
CGTATGGCTTTCATTTTCTCCTCCTTGTATAAATCCTGGTTGCTGTCTCT
TTATGAGGAGTTGTGGCCCGTTGTCAGGCAACGTGGCGTGGTGTGCACTG
TGTTTGCTGACGCAACCCCCACTGGTTGGGGCATTGCCACCACCTGTCAG
CTCCTTTCCGGGACTTTCGCTTTCCCCCTCCCTATTGCCACGGCGGAACT
CATCGCCGCCTGCCTTGCCCGCTGCTGGACAGGGGCTCGGCTGTTGGGCA
CTGACAATTCCGTGGTGTTGTCGGGGAAATCATCGTCCTTTCCTTGGCTG
CTCGCCTGTGTTGCCACCTGGATTCTGCGCGGGACGTCCTTCTGCTACAA
TCCAGCGGACCTTCCTTCCCGCGGCCTGCTGCCGGCTCTGCGGCCTCTTC
CGCGTCTTCGAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAACTATAGCCATACAATAAAAGTG
CGAAACGTTATCCTATAAGTAAGAAAGTTTTAAAATTTTCTTACGAAAAG
GATAGAACTTAAAAGTTCTAACTGTTCTACTAAAGTAATAAGTGAAAATC
TTATTTAAAGCAAACAACCAAGTAGCTTTAAGTCTAAGTCCCCTACACAA
GTTTTATACTACTATGCAAAACTTGTGAAGCTAGGTAAGGTCGTAATCCG
TGAAAGTCGGATGCGGGGCTCCTTAAAAGATTACTATGGTAAACATAAGC
TAATCCATTAAGATGCGATTTATATGTATTTTATACTGTTAAATATTTTT
GTGCTTGTGGCTTGGTATAAAACAGTTAAGATGAAGTACTTAACTGGTTT
TGGAATAATTGGTTGTTAAACTAAAACATTATAAATCGTTAGTGGATACC
TAAGGTAATCAAAAATAGGGATAGGTAGAATGGAACGTTTGATGCTGTAT
ATGAAGAGGTTTAGTAGAACCTAGGACACATATACGGGCTCAGCAGGTTC
ATAGTAGCTATGATACTCAGCCGGAAGTCAAATTAATTTTGAAATACTTC
TATGGTAACATAGGAGAAGGATAAAACTGAGTGAGCCAAGGAACCTAGTC
GGTAATAGCTGCCATCAGTCGGCGTGGACTGTAGAACACTGCCAATGCCG
GTCCCAAGCCCGGATAAAAGTGGAGGGTACAGTCCACGCTTTTTTTCTCG
AGTTAAGGGCGAATTCCCGATAAGGATCTTCCTAGAGCATGGCTACGTAG
ATAAGTAGCATGGCGGGTTAATCATTAACTACAAGGAACCCCTAGTGATG
GAGTTGGCCACTCCCTCTCTGCGCGCTCGCTCGCTCACTGAGGCCGGGCG
ACCAAAGGTCGCCCGACGCCCGGGCTTTGCCCGGGCGGCCTCAGTGAGCG
AGCGAGCGCGCAGCCTTAATTAACCTAATTCACTGGCCGTCGTTTTACAA
CGTCGTGACTGGGAAAACCCTGGCGTTACCCAACTTAATCGCCTTGCAGC
ACATCCCCCTTTCGCCAGCTGGCGTAATAGCGAAGAGGCCCGCACCGATC
GCCCTTCCCAACAGTTGCGCAGCCTGAATGGCGAATGGGACGCGCCCTGT
AGCGGCGCATTAAGCGCGGCGGGTGTGGTGGTTACGCGCAGCGTGACCGC
TACACTTGCCAGCGCCCTAGCGCCCGCTCCTTTCGCTTTCTTCCCTTCCT
TTCTCGCCACGTTCGCCGGCTTTCCCCGTCAAGCTCTAAATCGGGGGCTC
CCTTTAGGGTTCCGATTTAGTGCTTTACGGCACCTCGACCCCAAAAAACT
TGATTAGGGTGATGGTTCACGTAGTGGGCCATCGCCCTGATAGACGGTTT
TTCGCCCTTTGACGTTGGAGTCCACGTTCTTTAATAGTGGACTCTTGTTC
CAAACTGGAACAACACTCAACCCTATCTCGGTCTATTCTTTTGATTTATA
AGGGATTTTGCCGATTTCGGCCTATTGGTTAAAAAATGAGCTGATTTAAC
AAAAATTTAACGCGAATTTTAACAAAATATTAACGTTTATAATTTCAGGT
GGCATCTTTCGGGGAAATGTGCGCGGAACCCCTATTTGTTTATTTTTCTA
AATACATTCAAATATGTATCCGCTCATGAGACAATAACCCTGATAAATGC
TTCAATAATATTGAAAAAGGAAGAGTATGAGTATTCAACATTTCCGTGTC
GCCCTTATTCCCTTTTTTGCGGCATTTTGCCTTCCTGTTTTTGCTCACCC
AGA
pCMV-T7-PVX-Genome-
Sequence:
(SEQ ID NO: 30)
CTGCAGTAATACGACTCACTATAAGAGCCGAAAACTAAACCATACACCAC
CAACACAACCAAACCCACCACGCCCAATTGTTACACACCCGCTTGGAAAA
GCAAGTCTAACAAATGGCCAAAGTGCGCGAGGTTTACCAATCCTTTACAG
ACTCCACCACAAAAACTCTCATCCAAGATGAGGCTTATAGAAATATTCGC
CCCATCATGGAAAAACACAAACTAGCTAACCCTTACGCTCAAACGGTTGA
AGCGGCTAATGATCTAGAGGGGTTCGGCATAGCCACCAATCCCTATAGCA
TTGAATTGCATACACATGCAGCCGCTAAGACCATAGAGAATAAACTTCTA
GAGGTGCTTGGTTCCATCCTACCACAAGAACCTGTTACATTTATGTTCCT
TAAACCCAGGAAGCTAAACTACATGAGAAGAAACCCGCGGATCAAGGACA
TTTTCCACAATGTTGCCATTGAACCGAGAGACGTAGCAAGGTACCCCAAG
GAAACAATAATTGACAAACTCACAGAGATCACAACGGAAACAGCATACAT
TAGTGATACTCTGCACTTCTTGGATCCGAGCTACATAGTGGAGACATTCC
AAAACTGCCCAAAACTGCAAACATTGTATGCGACCTTAGTTCTCCCCGTT
GAGGCAGCCTTCAAAATGGAAAGCACTCACCCGAACATATACAGCCTCAA
ATACTTCGGAGATGGTTTCCAGTATATACCAGGCAACCATGGTGGTGGGG
CATACCATCATGAATTCGCTCATTTACAATGGCTCAAAGTGGGAAAGATC
AAGTGGAGGGACCCCAAGGATAGCTTTCTCGGACATCTCAATTACACGAC
TGAGCAGGTTGAGATGCACACAGTGACAGTACAGTTGCAGGAATCGTTTG
CGGCAAACCACTTGTACTGCATTAGGAGAGGAGACTTGCTCACACCGGAG
GTGCGTACTTTCGGCCAACCTGACAGGTATGTGATTCCACCACAGATCTT
TCTCCCAAAAGTTCACAACTGCAAGAAGCCGATTCTTAAGAAAACTATGA
TGCAGCTCTTCTTGTATGTTAGGACAGTCAAGGTCGCAAAAAATTGTGAC
ATCTTTGCCAAAGTCAGACAATTAATTAAATCATCTGACTTAGACAAATA
TTCTGCTGTGGAACTGGTTTACTTAGTAAGCTATATGGAGTTCCTTGCCG
ATTTACAAGCTACCACCTGCTTCTCAGACACACTCTCTGGTGGCTTGCTA
ACAAAGACCCTTGCACCGGTGAGGGCTTGGATACAAGAAAAGAAGATGCA
GCTGTTTGGTCTTGAGGACTACGCGAAGTTAGTCAAAGCAGTTGATTTCC
ACCCGGTGGATTTTTCTTTCAAAGTGGAAACTTGGGACTTCAGATTCCAC
CCCTTGCAAGCGTGGAAAGCCTTCCGACCAAGGGAAGTGTCGGATGTAGA
GGAAATGGAAAGTTTGTTCTCAGATGGGGACCTGCTTGATTGCTTCACAA
GAATGCCAGCTTATGCAGTAAACGCAGAGGAAGATTTAGCTACAATCAGG
AAAACGCCCGAGATGGATGTCGGTCAAGAAGTTAAAGAGCCTGCAGGAGA
CAGAAATCAATACTCAAACCCTGCAGAAACTTTCCTCAACAAGCTCCACA
GGAAACACAGTAGGGAGGTGAAACACCAGGCCGTAAAGAAAGCTAAACGC
CTAGCTGAAATCCAGGAGTCCATGAGAGCTGAAGGTGAGGCCGAACCAAA
TGAGACGAGCGGGGGCATGGGGGCAATACCCAGCAACGCCGAACTTCCCG
GCACGAGTGATGCCAGACAAGAACTCACACTCCCAACCACTAAACCTGTT
CCTGCAAGGTGGGAAGATGCTTCATTCACAGATTCTAGTGTGGAAGAGGA
GCAGGTAAGACTCCTTGGAGAAGAAGCAGTGAAAACAGCGACGCAGCAAG
TCATCGAAGGACTCCCTTGGAAACACTGGATTCCTCAACTAAATGCTGTT
GGATTCAAGGCGCTGTTAATCCAGAGGGATAGGAGTGGAACGATGATCAT
GCCCATCACAGAAATGGTCTCCGGGTTGGAAAAAGAGGACTTCCCGGAAG
GAACTCCAAAAGAGTTGGCACGAGAATTACTCGTTATGAACAGAAGCCCT
GCCACCATCCCTTTGGACCTGCTTAGAGCCAGAGACTACGGCAGTGATGT
AAAGAACAAGAGAATTGGTGCCATCACAAAGACACAGGCAACGAGTTGGG
GCGAGTACTTAACAGGAAAGATAGAAAGCCTGACTGAGAGGAAAGTTGCG
ACTTGTGTCATTCATGGAGCTGGAGGCTCTGGGAAAAGTCATGCCATCCA
GAAGGCACTGAGAGAAATTGGCAAGGGCTCGGACATCACTGTAGTCCTGC
CGACCAATGAACTGCGGCTAGATTGGAGTAAGAAGGTGCCTAACACTGAA
CCCTATATGTTCAAGACCTACGAAAAGGCGTTAATTGGGGGAACAGGCAG
CATAGTCATCTTTGACGATTACTCAAAACTTCCTCCCGGTTACATAGAAG
CCTTAGTCTGTTTCTACTCCAAAATCAAGCTAATCATTCTAACAGGAGAT
AGCAGACAGAGCGTCTACCATGAAACTGCTGAGGACGCCTCCATCAGGCA
TTTGGGGCCAGCGACAGAGTACTTCTCAAAATACTGCCGATACTATCTCA
ATGCTACACACCGCAACAAGAAAGACCTTGCGAACATGCTTGGTGTCTAC
AGTGAGAGAACGGGAGTCACTGAAATCAGCATGAGCGCCGAGTTTTTAGA
AGGAATCCCAACTTTAGTACCCTCGGATGAGAAGAGAAAGCTGTACATGG
GCACCGGGAGGAATGACACATTCACATACGCTGGATGCCAGGGGCTAACT
AAGCCGAAAGTACAAATAGTGTTGGACCACAACACCCAAGTGTGTAGTGC
GAATGTGATGTACACGGCACTTTCTAGAGCCACCGATAGGATTCACTTCG
TGAACACAAGTGCAAACTCCTCGGCCTTCTGGGAAAAGTTGGACAGCACC
CCTTACCTCAAGACTTTCCTATCAGTGGTGAGAGAACAAGCACTCAGGGA
GTATGAGCCGGCAGAGGCAGAGCCAATTCAAGAGCCTGAGCCCCAGACAC
ACATGTGTGTCGAGAATGAGGAGTCCGTGCTAGAAGAGTACAAAGAGGAA
CTCTTGGAAAAGTTTGACAGAGAGATACACTCTGAATCCCATGGTCATTC
AAACTGTGTCCAAACTGAAGACACAACCATTCAGTTGTTTTCGCATCAAC
AAGCAAAAGATGAGACCCTCCTCTGGGCGACCATAGATGCGCGGCTCAAG
ACTAGCAATCAAGAGGCAAACTTCCGAGAATTCCTGAGCAAGAAGGACAT
TGGGGACGTTCTGTTTTTAAACTACCAAAAAGCTATGGGTTTGCCCAAAG
AGCGTATTCCTTTTTCCCAAGAGGTCTGGGAAGCTTGTGCCCACGAAGTA
CAAAGCAAGTACCTCAGTAAGTCAAAGTGCAACTTGATCAATGGGACTGT
GAGACAGAGCCCAGACTTCGATGAAAACAAGATTATGGTATTCCTCAAGT
CGCAGTGGGTCACAAAGGTGGAAAAACTAGGTCTACCCAAGATTAAGCCA
GGTCAAACCATAGCAGCCTTTTACCAGCAGACTGTGATGCTTTTTGGAAC
TATGGCTAGGTACATGCGATGGTTCAGACAGGCTTTCCAGCCAAAAGAAG
TCTTCATAAACTGTGAGACTACGCCAGAAGACATGTCTGTATGGGCCTTG
AACAACTGGAATTTCAGCAGACCTAGCTTAGCTAATGACTACACAGCTTT
CGACCAGTCTCAGGATGGAGCTATGCTGCAATTTGAGGTGCTCAAAGCCA
AGCACCACTGCATACCAGAGGAAATCATCCAAGCATACATAGACATTAAG
ACCAATGCACAGATTTTCCTAGGCACATTATCGATTATGCGCCTGACTGG
TGAGGGTCCCACTTTTGATGCAAACACTGAGTGCAACATAGCTTACACCC
ACACAAAGTTTGACATCCCAGCCGGAACTGCTCAAGTTTATGCAGGAGAC
GACTCCGCACTGGATTGCGTTCCAGAAGTGAAGCATAGTTTCCACAGGCT
TGAAGACAAATTACTCCTCAAGTCAAAGCCTGTAATCACGCAGCAAAAGA
AAGGCAGTTGGCCTGAGTTTTGTGGTTGGCTGATCACACCAAAAGGGGTA
ATGAAAGACCCAATTAAGCTCCATGTTAGCTTAAAATTGGCCGAAGCTAA
GGGTGAACTCAAGAAATGTCAAGATTCCTATGAAATTGATCTGAGTTATG
CCTATGACCACAAGGACTCTCTGCATGACTTGTTCGATGAGAAACAGTGT
CAGGCACATACACTCACTTGCAGGACACTAATCAAGTCAGGGAGAGGCAC
TGTCTCACTTCCCCGCCTCAGAAACTTTCTTTAACCGTTAATTTACCTTA
TAGATTTGAATAAGATGGATATTCTCATCAGTAGTTTGAAAAGTTTAGGT
TATTCTAGGACTTCTAAATCTTTAGATTCAGGACCTTTGGTAGTACATGC
AGTAGCCGGAGCAGGTAAGTCCACAGCCCTAAGGAAGTTGATCCTCAGAC
ACCCAACATTCACCGTGCATACACTCGGTGTCCCTGACAAGGTGAGTATC
AGAACTAGAGGCATACAGAAGCCAGGACCTATTCCTGAGGGCAATTTCGC
AATCCTCGATGAGTATACTTTGGACAACACCACAAGGAACTCATACCAGG
CACTTTTTGCTGACCCTTATCAGGCACCTGAGTTTAGCCTAGAGCCCCAC
TTCTACTTGGAAACATCATTTCGAGTTCCGAGGAAAGTGGCAGATTTGAT
AGCTGGCTGTGGCTTCGATTTCGAGACTAACTCACAGGAAGAAGGGCATT
TAGAGATCACTGGCATATTCAAAGGGCCCCTACTTGGAAAGGTGATAGCC
ATTGATGAGGAGTCTGAGACAACACTGTCCAGGCATGGTGTTGAGTTTGT
TAAGCCCTGCCAAGTGACTGGACTTGAGTTGAAAGTAGTCACTATTGTGT
CTGCCGCACCAATAGAGGAAATTGGCCAGTCCACAGCTTTCTACAACGCT
ATCACCAGGTCAAAGGGATTGACATATGTCCGCGCAGGGACATAGACTGA
CCGCTCCGGTCAATTCTGAAAAAGTGTACATAGTATTAGGTCTATCATTT
GCTTTAGTTTCAATTACTTTCCTGCTTTCTAGAAATAGTTTGCCCCACGT
CGGTGACAACATTCACAGCTTGCCACACGGAGGAGCTTACAGAGACGGCA
CCAAAGCAATCTTGTACAACTCCCCAAATCTAGGGTCACGAGTGAGTCTA
CACAACGGAAAGAACGCAGCATTTGCTGCCGTTTTGCTACTGACTTTGCT
GATCTATGGAAGTAAATACATATCTCAACGCAATCATACTTGTGCTTGTG
GTAACAATCATAGCAGTCATTAGTACTTCCTTAGTGAGGACTGAACCTTG
TGTCATCAAGATTACTGGGGAATCAATCACAGTGTTGGCTTGCAAATTAG
ATGCAGAAACTATAAAAGCCATTGCCGATCTCAAGCCACTCTCCGTTGAA
CGGTTAAGTTTCCATTGATACTCGAAAGATGTCAGCACCAGCTAGCACAA
CACAGGCCACAGGGTCAACTACCTCAACTACCACGAAAACTGCAGGCGCA
ACTCCTGCCACAGCTTCAGGCCTGTTCACCATCCCGGATGGGGATTTCTT
TAGTACAGCTCGTGCCATAGTAGCCAGCAATGCTGTCGCAACAAATGAGG
ACCTCAGCAAGATTGAGGCTATTTGGAAGGACATGAAGGTGCCCACAGAC
ACTATGGCACAGGCTGCTTGGGACTTAGTCAGACACTGTGCTGATGTGGG
ATCATCTGCTCAAACAGAAATGATAGATACAGGTCCTTATTCCAACGGCA
TCAGCAGAGCTAGACTGGCAGCAGCAATCAAAGAGGTGTGCACACTTAGG
CAATTTTGCATGAAGTATGCTCCAGTGGTATGGAACTGGATGTTAACTAA
CAACAGTCCACCTGCTAACTGGCAAGCACAAGGTTTCAAGCCTGAGCACA
AATTCGCTGCATTCGACTTCTTCAATGGAGTCACCAACCCAGCTGCCATC
ATGCCCAAAGAGGGGCTCATCCGGCCACCGTCTGAAGCTGAAATGAATGC
TGCCCAAACTGCTGCTTTTGTGAAGATTACAAAGGCCAGGGCACAATCCA
ACGACTTTGCCAGCCTAGATGCAGCTGTCACTCGAGGTCGTATCACTGGA
ACAACAACCGCTGAGGCTGTTGTCACTCTACCACCACCATAACTACGTCT
ACATAACCGACGCCTACCCCAGTTTCATAGTATTTTCTGGTTTGATTGTA
TGAATAATATAAATTCTAGAGCTGGAGCCTCGGTAGCCGTTCCTCCTGCC
CGCTGGGCCTCCCAACGGGCCCTCCTCCCCTCCTTGCACCGGCCCTTCCT
GGTCTTTGCCGCGGCTGTGCCTTCTAGTTGCCAGCCATCTGTTGTTTGCC
CCTCCCCCGTGCCTTCCTTGACCCTGGAAGGTGCCACTCCCACTGTCCTT
TCCTAATAAAATGAGGAAATTGCATCGCATTGTCTGAGTAGGTGTCATTC
TATTCTGGGGGGTGGGGTGGGGCAGGACAGCAAGGGGGAGGATTGGGAAG
ACAATAGCAGGCATGCTGGGGATGCGGTGGGCTCTATGGTCGCCGGCGGT
CGACGTACCCAGCTTTTGTTCCCTTTAGTGAGGGTTAATTGCGCGCTTGG
CGTAATCATGGTCATAGCTGTTTCCTGTGTGAAATTGTTATCCGCTCACA
ATTCCACACAACATACGAGCCGGAAGCATAAAGTGTAAAGCCTGGGGTGC
CTAATGAGTGAGCTAACTCACATTAATTGCGTTGCGCTCACTGCCCGCTT
TCCAGTCGGGAAACCTGTCGTGCCAGCTGCATTAATGAATCGGCCAACGC
GCGGGGAGAGGCGGTTTGCGTATTGGGCGCTCTTCCGCTTCCTCGCTCAC
TGACTCGCTGCGCTCGGTCGTTCGGCTGCGGCGAGCGGTATCAGCTCACT
CAAAGGCGGTAATACGGTTATCCACAGAATCAGGGGATAACGCAGGAAAG
AACATGTGAGCAAAAGGCCAGCAAAAGGCCAGGAACCGTAAAAAGGCCGC
GTTGCTGGCGTTTTTCCATAGGCTCCGCCCCCCTGACGAGCATCACAAAA
ATCGACGCTCAAGTCAGAGGTGGCGAAACCCGACAGGACTATAAAGATAC
CAGGCGTTTCCCCCTGGAAGCTCCCTCGTGCGCTCTCCTGTTCCGACCCT
GCCGCTTACCGGATACCTGTCCGCCTTTCTCCCTTCGGGAAGCGTGGCGC
TTTCTCATAGCTCACGCTGTAGGTATCTCAGTTCGGTGTAGGTCGTTCGC
TCCAAGCTGGGCTGTGTGCACGAACCCCCCGTTCAGCCCGACCGCTGCGC
CTTATCCGGTAACTATCGTCTTGAGTCCAACCCGGTAAGACACGACTTAT
CGCCACTGGCAGCAGCCACTGGTAACAGGATTAGCAGAGCGAGGTATGTA
GGCGGTGCTACAGAGTTCTTGAAGTGGTGGCCTAACTACGGCTACACTAG
AAGGACAGTATTTGGTATCTGCGCTCTGCTGAAGCCAGTTACCTTCGGAA
AAAGAGTTGGTAGCTCTTGATCCGGCAAACAAACCACCGCTGGTAGCGGT
GGTTTTTTTGTTTGCAAGCAGCAGATTACGCGCAGAAAAAAAGGATCTCA
AGAAGATCCTTTGATCTTTTCTACGGGGTCTGACGCTCAGTGGAACGAAA
ACTCACGTTAAGGGATTTTGGTCATGAGATTATCAAAAAGGATCTTCACC
TAGATCCTTTTAAATTAAAAATGAAGTTTTAAATCAATCTAAAGTATATA
TGAGTAAACTTGGTCTGACAGTTACCAATGCTTAATCAGTGAGGCACCTA
TCTCAGCGATCTGTCTATTTCGTTCATCCATAGTTGCCTGACTCCCCGTC
GTGTAGATAACTACGATACGGGAGGGCTTACCATCTGGCCCCAGTGCTGC
AATGATACCGCGAGACCCACGCTCACCGGCTCCAGATTTATCAGCAATAA
ACCAGCCAGCCGGAAGGGCCGAGCGCAGAAGTGGTCCTGCAACTTTATCC
GCCTCCATCCAGTCTATTAATTGTTGCCGGGAAGCTAGAGTAAGTAGTTC
GCCAGTTAATAGTTTGCGCAACGTTGTTGCCATTGCTACAGGCATCGTGG
TGTCACGCTCGTCGTTTGGTATGGCTTCATTCAGCTCCGGTTCCCAACGA
TCAAGGCGAGTTACATGATCCCCCATGTTGTGCAAAAAAGCGGTTAGCTC
CTTCGGTCCTCCGATCGTTGTCAGAAGTAAGTTGGCCGCAGTGTTATCAC
TCATGGTTATGGCAGCACTGCATAATTCTCTTACTGTCATGCCATCCGTA
AGATGCTTTTCTGTGACTGGTGAGTACTCAACCAAGTCATTCTGAGAATA
GTGTATGCGGCGACCGAGTTGCTCTTGCCCGGCGTCAATACGGGATAATA
CCGCGCCACATAGCAGAACTTTAAAAGTGCTCATCATTGGAAAACGTTCT
TCGGGGCGAAAACTCTCAAGGATCTTACCGCTGTTGAGATCCAGTTCGAT
GTAACCCACTCGTGCACCCAACTGATCTTCAGCATCTTTTACTTTCACCA
GCGTTTCTGGGTGAGCAAAAACAGGAAGGCAAAATGCCGCAAAAAAGGGA
ATAAGGGCGACACGGAAATGTTGAATACTCATACTCTTCCTTTTTCAATA
TTATTGAAGCATTTATCAGGGTTATTGTCTCATGAGCGGATACATATTTG
AATGTATTTAGAAAAATAAACAAATAGGGGTTCCGCGCACATTTCCCCGA
AAAGTGCCACCTAAATTGTAAGCGTTAATATTTTGTTAAAATTCGCGTTA
AATTTTTGTTAAATCAGCTCATTTTTTAACCAATAGGCCGAAATCGGCAA
AATCCCTTATAAATCAAAAGAATAGACCGAGATAGGGTTGAGTGTTGTTC
CAGTTTGGAACAAGAGTCCACTATTAAAGAACGTGGACTCCAACGTCAAA
GGGCGAAAAACCGTCTATCAGGGCGATGGCCCACTACGTGAACCATCACC
CTAATCAAGTTTTTTGGGGTCGAGGTGCCGTAAAGCACTAAATCGGAACC
CTAAAGGGAGCCCCCGATTTAGAGCTTGACGGGGAAAGCCGGCGAACGTG
GCGAGAAAGGAAGGGAAGAAAGCGAAAGGAGCGGGCGCTAGGGCGCTGGC
AAGTGTAGCGGTCACGCTGCGCGTAACCACCACACCCGCCGCGCTTAATG
CGCCGCTACAGGGCGCGTCCCATTCGCCATTCAGGCTGCGCAACTGTTGG
GAAGGGCGATCGGTGCGGGCCTCTTCGCTATTACGCCAGCTGGCGAAAGG
GGGATGTGCTGCAAGGCGATTAAGTTGGGTAACGCCAGGGTTTTCCCAGT
CACGACGTTGTAAAACGACGGCCAGTGAGCG
pHGWA-9xHis-PVX-CP-
Sequence:
(SEQ ID NO: 31)
AGATCCAGATCTCGATCCCGCGAAATTAATACGACTCACTATAGGGGAAT
TGTGAGCGGATAACAATTCCCCTCTAGAAATAATTTTGTTTAACTTTAAG
AAGGAGATATACCATGGGCAGCAGCCATCATCATCATCATCATCATCATC
ACGGTGAGAATCTTTATTTTCAGGGCATGTCAGCACCAGCTAGCACAACA
CAGACCATAGGGTCAACTACCTCAACTACCACAAAAACTGCAGGCGCAAC
TCCTGCCACAGCTTCAGGACTGTTCACCATCCCGGATGGGGATTTCTTTA
ACACAGCCCGTGCCATAGTAGCCAGCAATGCCGTTGCAACGAATGAGGAC
CTCAGAAAGATTGAGGCTATTTGGAAAGACATGAAGGTACCCACAGACAC
TATGGCACAGGCTGCTTGGGACTTGGTCAGACACTGTGCTGATGTGGGCT
CATCTGCCCAAACAGAGATGATAGATACAGGTCCTTATTCCAATGGCATC
AGCAGAGCTAGACTGGCAGCAGCGATTAAAGAGGTGTGCACACTTAGACA
ATTCTGCATGAAGTATGCCCCAGTGGTATGGAACTGGATGTTGACTAACA
ACAGTCCACCTGCTAACTGGCAAGCACAAGGTTTCAAGCCTGAGCACAAA
TTCGCTGCATTCGACTTCTTCAACGGAGTCACCAACCCAGCTGCCATCAT
GCCCAAAGAGGGACTCATCCGGCCACCATCTGAAGCAGAAATGAATGCTG
CCCAAACTGCTGCCTTTGTGAAAATTACGAAGGCCAGGGCACAATCCAAC
GACTTTGCCAGCCTAGATGCGGCTGTCACTCGAGGTCGTATCACTGGAAC
AACAACCGCTGAGGCTGTTGTCACTCTACCACCACCATAAAAGGGGGGCG
CGCCGACCCAGCTTTCTTGTACAAAGTGGTGATGTACCTCGAGCACCACC
ACCACCACCACTGAGATCCGGCTGCTAACAAAGCCCGAAAGGAAGCTGAG
TTGGCTGCTGCCACCGCTGAGCAATAACTAGCATAACCCCTTGGGGCCTC
TAAACGGGTCTTGAGGGGTTTTTTGCTGAAAGGAGGAACTATATCCGGAT
TGGCGAATGGGACGCGCCCTGTAGCGGCGCATTAAGCGCGGCGGGTGTGG
TGGTTACGCGCAGCGTGACCGCTACACTTGCCAGCGCCCTAGCGCCCGCT
CCTTTCGCTTTCTTCCCTTCCTTTCTCGCCACGTTCGCCGGCTTTCCCCG
TCAAGCTCTAAATCGGGGGCTCCCTTTAGGGTTCCGATTTAGTGCTTTAC
GGCACCTCGACCCCAAAAAACTTGATTAGGGTGATGGTTCACGTAGTGGG
CCATCGCCCTGATAGACGGTTTTTCGCCCTTTGACGTTGGAGTCCACGTT
CTTTAATAGTGGACTCTTGTTCCAAACTGGAACAACACTCAACCCTATCT
CGGTCTATTCTTTTGATTTATAAGGGATTTTGCCGATTTCGGCCTATTGG
TTAAAAAATGAGCTGATTTAACAAAAATTTAACGCGAATTTTAACAAAAT
ATTAACGTTTACAATTTCAGGTGGCACTTTTCGGGGAAATGTGCGCGGAA
CCCCTATTTGTTTATTTTTCTAAATACATTCAAATATGTATCCGCTCATG
AGACAATAACCCTGATAAATGCTTCAATAATATTGAAAAAGGAAGAGTAT
GAGTATTCAACATTTCCGTGTCGCCCTTATTCCCTTTTTTGCGGCATTTT
GCCTTCCTGTTTTTGCTCACCCAGAAACGCTGGTGAAAGTAAAAGATGCT
GAAGATCAGTTGGGTGCACGAGTGGGTTACATCGAACTGGATCTCAACAG
CGGTAAGATCCTTGAGAGTTTTCGCCCCGAAGAACGTTTTCCAATGATGA
GCACTTTTAAAGTTCTGCTATGTGGCGCGGTATTATCCCGTATTGACGCC
GGGCAAGAGCAACTCGGTCGCCGCATACACTATTCTCAGAATGACTTGGT
TGAGTACTCACCAGTCACAGAAAAGCATCTTACGGATGGCATGACAGTAA
GAGAATTATGCAGTGCTGCCATAACCATGAGTGATAACACTGCGGCCAAC
TTACTTCTGACAACGATCGGAGGACCGAAGGAGCTAACCGCTTTTTTGCA
CAACATGGGGGATCATGTAACTCGCCTTGATCGTTGGGAACCGGAGCTGA
ATGAAGCCATACCAAACGACGAGCGTGACACCACGATGCCTGCAGCAATG
GCAACAACGTTGCGCAAACTATTAACTGGCGAACTACTTACTCTAGCTTC
CCGGCAACAATTAATAGACTGGATGGAGGCGGATAAAGTTGCAGGACCAC
TTCTGCGCTCGGCCCTTCCGGCTGGCTGGTTTATTGCTGATAAATCTGGA
GCCGGTGAGCGTGGGTCTCGCGGTATCATTGCAGCACTGGGGCCAGATGG
TAAGCCCTCCCGTATCGTAGTTATCTACACGACGGGGAGTCAGGCAACTA
TGGATGAACGAAATAGACAGATCGCTGAGATAGGTGCCTCACTGATTAAG
CATTGGTAACTGTCAGACCAAGTTTACTCATATATACTTTAGATTGATTT
AAAACTTCATTTTTAATTTAAAAGGATCTAGGTGAAGATCCTTTTTGATA
ATCTCATGACCAAAATCCCTTAACGTGAGTTTTCGTTCCACTGAGCGTCA
GACCCCGTAGAAAAGATCAAAGGATCTTCTTGAGATCCTTTTTTTCTGCG
CGTAATCTGCTGCTTGCAAACAAAAAAACCACCGCTACCAGCGGTGGTTT
GTTTGCCGGATCAAGAGCTACCAACTCTTTTTCCGAAGGTAACTGGCTTC
AGCAGAGCGCAGATACCAAATACTGTCCTTCTAGTGTAGCCGTAGTTAGG
CCACCACTTCAAGAACTCTGTAGCACCGCCTACATACCTCGCTCTGCTAA
TCCTGTTACCAGTGGCTGCTGCCAGTGGCGATAAGTCGTGTCTTACCGGG
TTGGACTCAAGACGATAGTTACCGGATAAGGCGCAGCGGTCGGGCTGAAC
GGGGGGTTCGTGCACACAGCCCAGCTTGGAGCGAACGACCTACACCGAAC
TGAGATACCTACAGCGTGAGCTATGAGAAAGCGCCACGCTTCCCGAAGGG
AGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCG
GGTTTCGCCACCTCTGACTTGAGCGTCGATTTTTGTGATGCTCGTCAGGG
GGGCGGAGCCTATGGAAAAACGCCAGCAACGCGGCCTTTTTACGGTTCCT
GGCCTTTTGCTGGCCTTTTGCTCACATGTTCTTTCCTGCGTTATCCCCTG
ATTCTGTGGATAACCGTATTACCGCCTTTGAGTGAGCTGATACCGCTCGC
CGCAGCCGAACGACCGAGCGCAGCGAGTCAGTGAGCGAGGAAGCGGAAGA
GCGCCTGATGCGGTATTTTCTCCTTACGCATCTGTGCGGTATTTCACACC
GCATATATGGTGCACTCTCAGTACAATCTGCTCTGATGCCGCATAGTTAA
GCCAGTATACACTCCGCTATCGCTACGTGACTGGGTCATGGCTGCGCCCC
GACACCCGCCAACACCCGCTGACGCGCCCTGACGGGCTTGTCTGCTCCCG
GCATCCGCTTACAGACAAGCTGTGACCGTCTCCGGGAGCTGCATGTGTCA
GAGGTTTTCACCGTCATCACCGAAACGCGCGAGGCAGCTGCGGTAAAGCT
CATCAGCGTGGTCGTGAAGCGATTCACAGATGTCTGCCTGTTCATCCGCG
TCCAGCTCGTTGAGTTTCTCCAGAAGCGTTAATGTCTGGCTTCTGATAAA
GCGGGCCATGTTAAGGGCGGTTTTTTCCTGTTTGGTCACTGATGCCTCCG
TGTAAGGGGGATTTCTGTTCATGGGGGTAATGATACCGATGAAACGAGAG
AGGATGCTCACGATACGGGTTACTGATGATGAACATGCCCGGTTACTGGA
ACGTTGTGAGGGTAAACAACTGGCGGTATGGATGCGGCGGGACCAGAGAA
AAATCACTCAGGGTCAATGCCAGCGCTTCGTTAATACAGATGTAGGTGTT
CCACAGGGTAGCCAGCAGCATCCTGCGATGCAGATCCGGAACATAATGGT
GCAGGGCGCTGACTTCCGCGTTTCCAGACTTTACGAAACACGGAAACCGA
AGACCATTCATGTTGTTGCTCAGGTCGCAGACGTTTTGCAGCAGCAGTCG
CTTCACGTTCGCTCGCGTATCGGTGATTCATTCTGCTAACCAGTAAGGCA
ACCCCGCCAGCCTAGCCGGGTCCTCAACGACAGGAGCACGATCATGCGCA
CCCGTGGGGCCGCCATGCCGGCGATAATGGCCTGCTTCTCGCCGAAACGT
TTGGTGGCGGGACCAGTGACGAAGGCTTGAGCGAGGGCGTGCAAGATTCC
GAATACCGCAAGCGACAGGCCGATCATCGTCGCGCTCCAGCGAAAGCGGT
CCTCGCCGAAAATGACCCAGAGCGCTGCCGGCACCTGTCCTACGAGTTGC
ATGATAAAGAAGACAGTCATAAGTGCGGCGACGATAGTCATGCCCCGCGC
CCACCGGAAGGAGCTGACTGGGTTGAAGGCTCTCAAGGGCATCGGTCGAG
ATCCCGGTGCCTAATGAGTGAGCTAACTTACATTAATTGCGTTGCGCTCA
CTGCCCGCTTTCCAGTCGGGAAACCTGTCGTGCCAGCTGCATTAATGAAT
CGGCCAACGCGCGGGGAGAGGCGGTTTGCGTATTGGGCGCCAGGGTGGTT
TTTCTTTTCACCAGTGAGACGGGCAACAGCTGATTGCCCTTCACCGCCTG
GCCCTGAGAGAGTTGCAGCAAGCGGTCCACGCTGGTTTGCCCCAGCAGGC
GAAAATCCTGTTTGATGGTGGTTAACGGCGGGATATAACATGAGCTGTCT
TCGGTATCGTCGTATCCCACTACCGAGATATCCGCACCAACGCGCAGCCC
GGACTCGGTAATGGCGCGCATTGCGCCCAGCGCCATCTGATCGTTGGCAA
CCAGCATCGCAGTGGGAACGATGCCCTCATTCAGCATTTGCATGGTTTGT
TGAAAACCGGACATGGCACTCCAGTCGCCTTCCCGTTCCGCTATCGGCTG
AATTTGATTGCGAGTGAGATATTTATGCCAGCCAGCCAGACGCAGACGCG
CCGAGACAGAACTTAATGGGCCCGCTAACAGCGCGATTTGCTGGTGACCC
AATGCGACCAGATGCTCCACGCCCAGTCGCGTACCGTCTTCATGGGAGAA
AATAATACTGTTGATGGGTGTCTGGTCAGAGACATCAAGAAATAACGCCG
GAACATTAGTGCAGGCAGCTTCCACAGCAATGGCATCCTGGTCATCCAGC
GGATAGTTAATGATCAGCCCACTGACGCGTTGCGCGAGAAGATTGTGCAC
CGCCGCTTTACAGGCTTCGACGCCGCTTCGTTCTACCATCGACACCACCA
CGCTGGCACCCAGTTGATCGGCGCGAGATTTAATCGCCGCGACAATTTGC
GACGGCGCGTGCAGGGCCAGACTGGAGGTGGCAACGCCAATCAGCAACGA
CTGTTTGCCCGCCAGTTGTTGTGCCACGCGGTTGGGAATGTAATTCAGCT
CCGCCATCGCCGCTTCCACTTTTTCCCGCGTTTTCGCAGAAACGTGGCTG
GCCTGGTTCACCACGCGGGAAACGGTCTGATAAGAGACACCGGCATACTC
TGCGACATCGTATAACGTTACTGGTTTCACATTCACCACCCTGAATTGAC
TCTCTTCCGGGCGCTATCATGCCATACCGCGAAAGGTTTTGCGCCATTCG
ATGGTGTCCGGGATCTCGACGCTCTCCCTTATGCGACTCCTGCATTAGGA
AGCAGCCCAGTAGTAGGTTGAGGCCGTTGAGCACCGCCGCCGCAAGGAAT
GGTGCATGCAAGGAGATGGCGCCCAACAGTCCCCCGGCCACGGGGCCTGC
CACCATACCCACGCCGAAACAAGCGCTCATGAGCCCGAAGTGGCGAGCCC
GATCTTCCCCATCGGTGATGTCGGCGATATAGGCGCCAGCAACCGCACCT
GTGGCGCCGGTGATGCCGGCCACGATGCGTCCGGCGTAGAGGATCG
Circ-EMCV-GFP-SL1-
Sequence
(SEQ ID NO: 32)
aacgctggtgaaagtaaaagatgctgaagatcagttgggtgcacgagtgg
gttacatcgaactggatctcaatagtggtaagatccttgagagttttcgc
cccgaagaacgttttccaatgatgagcacttttaaagttctgctatgtgg
cgcggtattatcccgtattgacgccgggcaagagcaactcggtcgccgca
tacactattctcagaatgacttggttgagtactcaccagtcacagaaaag
catcttacggatggcatgacagtaagagaattatgcagtgctgccataac
catgagtgataacactgcggccaacttacttctgacaacgatcggaggac
cgaaggagctaaccgcttttttgcacaacatgggggatcatgtaactcgc
cttgatcgttgggaaccggagctgaatgaagccataccaaacgacgagcg
tgacaccacgatgcctgtagtaatggtaacaacgttgcgcaaactattaa
ctggcgaactacttactctagcttcccggcaacaattaatagactggatg
gaggcggataaagttgcaggaccacttctgcgctcggcccttccggctgg
ctggtttattgctgataaatctggagccggtgagcgtgggtctcgcggta
tcattgcagcactggggccagatggtaagccccccgtatcgtagttatct
acacgacggggagtcaggcaactatggatgaacgaaatagacagatcgct
gagataggtgcctcactgattaagcattggtaactgtcagaccaagttta
ctcatatatactttagattgatttaaaacttcatttttaatttaaaagga
tctaggtgaagatcctttttgataatctcatgaccaaaatcccttaacgt
gagttttcgttccactgagcgtcagaccccgtagaaaagatcaaaggatc
ttcttgagatcctttttttctgcgcgtaatctgctgcttgcaaacaaaaa
aaccaccgctaccagcggtggtttgtttgccggatcaagagctaccaact
ctttttccgaaggtaactggcttcagcagagcgcagataccaaatactgt
ccttctagtgtagccgtagttaggccaccacttcaagaactctgtagcac
cgcctacatacctcgctctgctaatcctgttaccagtggctgctgccagt
ggcgataagtcgtgtcttaccgggttggactcaagacgatagttaccgga
taaggcgcagcggtcgggctgaacggggggttcgtgcacacagcccagct
tggagcgaacgacctacaccgaactgagatacctacagcgtgagctatga
gaaagcgccacgcttcccgaagggagaaaggcggacaggtatccggtaag
cggcagggtcggaacaggagagcgcacgagggagcttccagggggaaacg
cctggtatctttatagtcctgtcgggtttcgccacctctgacttgagcgt
cgatttttgtgatgctcgtcaggggggcggagcctatggaaaaacgccag
caacgcggcctttttacggttcctggccttttgctgcggttttgctcaca
tgttctttcctgcgttatcccctgattctgtggataaccgtattaccgcc
tttgagtgagctgataccgctcgccgcagccgaacgaccgagcgcagcga
gtcagtgagcgaggaagcggaagagcgcccaatacgcaaaccgcctctcc
ccgcgcgttggccgattcattaatgcagctggcacgacaggtttcccgac
tggaaagcgggcagtgagcgcaacgcaattaatgtgagttagctcactca
ttaggcaccccaggctttacactttatgcttccggctcgtatgttgtgtg
gaattgtgagcggataacaatttcacacaggaaacagctatgaccatgat
tacgccagatttaattaaggctgcgcgctcgctcgctcactgaggccgcc
cgggcaaagcccgggcgtcgggcgacctttggtcgcccggcctcagtgag
cgagcgagcgcgcagagagggagtggccaactccatcactaggggttcct
tgtagttaatgattaacccgccatgctacttatctacgtagccatgctct
aggaagatcggaattcgcccttaagctagcatgcTAATACGACTCACTAT
AGGGCCATCAGTCGCCGGTCCCAAGCCCGGATAAAATGGGAGGGGGCGGG
AAACCGCCTAACCATGCCGACTGATGGCAGctattatcgagcgaacgcct
tatgcgatgaaagtcgcacgtagggtgtagaccaagcgaaatcctatgca
tttaggatagtgaggtatagcaaaggagaagtcgacgggcccgeggaatt
ccgccccccccccctctccctcccccccccctaacgttactggccgaagc
cgcttggaataaggccggtgtgcgtttgtctatatgttattttccaccat
attgccgtcttttggcaatgtgagggcccggaaacctggccctgtcttct
tgacgagcattcctaggggtctttcccctctcgccaaaggaatgcaaggt
ctgttgaatgtcgtgaaggaagcagttcctctggaagcttcttgaagaca
aacaacgtctgtagcgaccctttgcaggcagcggaaccccccacctggcg
acaggtgcctctgcggccaaaagccacgtgtataagatacacctgcaaag
gcggcacaaccccagtgccacgttgtgagttggatagttgtggaaagagt
caaatggctctcctcaagcgtattcaacaaggggctgaaggatgcccaga
aggtaccccattgtatgggatctgatctggggcctcggtgcacatgcttt
acatgtgtttagtcgaggttaaaaaacgtctaggccccccgaaccacggg
gacgtggttttcctttgaaaaacacgatgataatatggccacaaccatgg
tgagcaagggcgaggagctgttcaccggggtggtgcccatcctggtcgag
ctggacggcgacgtaaacggccacaagttcagcgtgtccggcgagggcga
gggcgatgccacctacggcaagctgaccctgaagttcatctgcaccaccg
gcaagctgcccgtgccctggcccaccctcgtgaccaccctgacctacggc
gtgcagtgcttcagccgctaccccgaccacatgaagcagcacgacttctt
caagtccgccatgcccgaaggctacgtccaggagcgcaccatcttcttca
aggacgacggcaactacaagacccgcgccgaggtgaagttcgagggcgac
accctggtgaaccgcatcgagctgaagggcatcgacttcaaggaggacgg
caacatcctggggcacaagctggagtacaactacaacagccacaacgtct
atatcatggccgacaagcagaagaacggcatcaaggtgaacttcaagatc
cgccacaacatcgaggacggcagcgtgcagctcgccgaccactaccagca
gaacacccccateggcgacggccccgtgctgctgcccgacaaccactacc
tgagcacccagtccgccctgagcaaagaccccaacgagaagcgcgatcac
atggtcctgctggagttcgtgaccgccgccgggatcactctcggcatgga
cgagctgtacaagtaaTTGTTACACACCCGCTTGAAAAAGCAAGTCTGAC
AAAAGGCCAAAGTGCGCGAGGgccacggatccaatcaacctctggattac
aaaatttgtgaaagattgactggtattcttaactatgttgctccttttac
gctatgtggatacgctgctttaatgcctttgtatcatgctattgcttccc
gtatggctttcattttctcctccttgtataaatcctggttgctgtctctt
tatgaggagttgtggcccgttgtcaggcaacgtggcgtggtgtgcactgt
gtttgctgacgcaacccccactggttggggcattgccaccacctgtcagc
tcctttccgggactttcgctttccccctccctattgccacggcggaactc
atcgccgcctgccttgcccgctgctggacaggggctcggctgttgggcac
tgacaattccgtggtgttgtcggggaaatcatcgtcctttccttggctgc
tcgcctgtgttgccacctggattctgcgcgggacgtccttctgctacaat
ccagcggaccttccttcccgcggcctgctgccggctctgcggcctcttcc
gcgtcttcgaaAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAactatagccatacaataaaagtgc
gaaacgttatcctataagtaagaaagttttaaaattttcttacgaaaagg
atagaacttaaaagttctaactgttctactaaagtaataagtgaaaatct
tatttaaagcaaacaaccaagtagctttaagtctaagtcccctacacaag
ttttatactactatgcaaaacttgtgaagctaggtaaggtcgtaatccgt
gaaagtcggatgcggggctccttaaaagattactatggtaaacataagct
aatccattaagatgcgatttatatgtattttatactgttaaatatttttg
tgcttgtggcttggtataaaacagttaagatgaagtacttaactggtttt
ggaataattggttgttaaactaaaacattataaatcgttagtggatacct
aaggtaatcaaaaatagggataggtagaatggaacgtttgatgctgtata
tgaagaggtttagtagaacctaggacacatatacgggctcagcaggttca
tagtagctatgatactcagccggaagtcaaattaattttgaaatacttct
atggtaacataggagaaggataaaactgagtgagccaaggaacctagtcg
gtaatagCTGCCATCAGTCGGCGTGGACTGTAGAACACTGCCAATGCCGG
TCCCAAGCCCGGATAAAAGTGGAGGGTACAGTCCACGCtttttttctcga
gttaagggcgaattcccgataaggatcttcctagagcatggctacgtaga
taagtagcatgggggttaatcattaactacaaggaacccctagtgatgga
gttggccactccctctctgcgcgctcgctcgctcactgaggccgggcgac
caaaggtcgcccgacgcccgggctttgcccgggcggcctcagtgagcgag
cgagcgcgcagccttaattaacctaattcactggccgtcgttttacaacg
tcgtgactgggaaaaccctggcgttacccaacttaatcgccttgcagcac
atccccctttcgccagctggcgtaatagcgaagaggcccgcaccgategc
ccttcccaacagttgcgcagcctgaatggcgaatgggacgcgccctgtag
cggcgcattaagcgcggcgggtgtggtggttacgcgcagcgtgaccgcta
cacttgccagcgccctagcgcccgctcctttcgctttcttcccttccttt
ctcgccacgttegccggctttccccgtcaagctctaaatcgggggctccc
tttagggttccgatttagtgctttacggcacctcgaccccaaaaaacttg
attagggtgatggttcacgtagtgggccatcgccctgatagacggttttt
cgccctttgacgttggagtccacgttctttaatagtggactcttgttcca
aactggaacaacactcaaccctatctcggtctattcttttgatttataag
ggattttgccgatttcggcctattggttaaaaaatgagctgatttaacaa
aaatttaacgcgaattttaacaaaatattaacgtttataatttcaggtgg
catctttcggggaaatgtgcgcggaacccctatttgtttatttttctaaa
tacattcaaatatgtatccgctcatgagacaataaccctgataaatgctt
caataatattgaaaaaggaagagtatgagtattcaacatttccgtgtcgc
ccttattcccttttttgcggcattttgccttcctgtttttgctcacccag
a

### EQUIVALENTS

Unless otherwise defined, all technical and scientific terms used herein have the same meaning as commonly understood by one of ordinary skill in the art to which this technology belongs.

The present technology illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### CLAUSES

Clause 1. A ring-shaped or halo-shaped nanoparticle comprising a filamentous plant virus coat protein, optionally wherein the filamentous plant virus coat protein is from Tobacco Mosaic Virus (TMV), Potato Virus X (PVX), Alternanthera mosaic virus, Papaya mosaic virus, or another member of the Potexvirus or Tobamovirus genera, the coat protein being assembled on one or more circular RNA (circRNA), optionally wherein the one or more circRNA are the same or different from each other.

Clause 2. The ring-shaped or halo-shaped nanoparticle of clause 1, wherein the filamentous plant virus coat protein is from Tobacco Mosaic Virus (TMV) or Potato Virus X (PVX).

Clause 3. The ring-shaped or halo-shaped nanoparticle of clause 1 or 2, wherein the coat protein comprises chemically modified lysine, cysteine, or histidine residues to permit conjugation of a therapeutic, diagnostic, or targeting moiety.

Clause 4. The ring-shaped or halo-shaped nanoparticle of any one of clauses 1-3, wherein the targeting moiety is selected from an RGD peptide, an antibody or antibody fragment, an aptamer, a ligand for a cell-surface receptor, or a carbohydrate.

Clause 5. The ring-shaped or halo-shaped nanoparticle of any one of clauses 1-4, wherein the RNA cargo is a self-amplifying RNA (replicon) encoding an RNA-dependent RNA polymerase (RdRp) and a gene of interest separated by a self-cleaving peptide sequence.

Clause 6. The ring-shaped nanoparticle of clause 5, wherein the RNA-dependent RNA polymerase is derived from an alphavirus or a nodavirus.

Clause 7. The ring-shaped nanoparticle of any one of clauses 3-6, wherein the coat protein subunits are crosslinked with a bifunctional or multifunctional crosslinker, optionally a polyethylene glycol (PEG) crosslinker and/or comprises a membrane-active, pH-sensitive fusogenic peptide.

Clause 8. The ring-shaped or halo-shaped nanoparticle of clause 7, wherein the crosslinker is an NHS-ester-PEG or maleimide-PEG targeting lysine or cysteine residues of the coat protein.

Clause 9. The ring-shaped or halo-shaped nanoparticle of clause 7 or 8, wherein the crosslinking increases rupture force and rupture distance of the nanoparticle compared to an unmodified nanoparticle, optionally as measured by single-molecule force spectroscopy.

Clause 10. The ring-shaped or halo-shaped nanoparticle of any one of clauses 1-3, further comprising a plurality of coat proteins and crosslinkers linking the plurality of coat proteins, wherein the plurality of the coat proteins are assembled on the one or more of circRNA, optionally wherein the one or more circRNA and/or the plurality of plant virus coat proteins are the same or different from each other, and further optionally wherein the plurality of PVX coat proteins are crosslinked by polyethylene glycol crosslinkers, and further optionally wherein the polyethylene glycol crosslinkers bind to a lysine or a cysteine on the coat protein.

Clause 11. The ring-shaped or halo-shaped nanoparticle of any one of clauses 1-10, further comprising a circRNA cassette comprising the one or more circRNA.

Clause 12. The ring-shaped or halo-shaped nanoparticle of clause 11, wherein the circRNA cassette comprises an internal ribosome entry site (IRES), optionally further comprising an origin of assembly site (OAS), and optionally further comprising a RNA-dependent RNA polymerase (RdRp).

Clause 13. The ring-shaped or halo-shaped nanoparticle of any one of clauses 1-12, wherein the one or more circRNA encodes one or more of: a cytokine, an immunomodulatory molecule, or a reporter protein, optionally wherein the cytokine is selected from GM-CSF, TNFα, IFN-7, TGF-β, IL-2, IL-4, IL-10 and IL-13.

Clause 14. The ring-shaped or halo-shaped nanoparticle any one of clauses 1-13, further comprising an RNA-binding domain in the PVX coat protein, optionally wherein the RNA-binding domain is selected from a HK-domain, zinc-finger domain, or a peptide selected through phage display.

Clause 15. The ring-shaped or halo-shaped nanoparticle of any one of clauses 1-14, wherein the coat protein comprises a membrane-active, pH-sensitive fusogenic peptide.

Clause 16. The ring-shaped or halo-shaped nanoparticle of any one of clauses 1-15, further comprising a detectable marker.

Clause 17. The ring-shaped or halo-shaped nanoparticle of any one of clauses 1-16, wherein the circumference of the nanoparticle is about 200 to about 2000 nanometers in circumference.

Clause 18. The ring-shaped or halo-shaped nanoparticle of any one of clauses 1-17, wherein the coat protein was recombinantly expressed in E. coli, yeast, or another heterologous host, optionally comprising an N-terminal histidine tag.

Clause 19. The ring-shaped or halo-shaped nanoparticle of any one of clauses 1-18, further comprising chemically or genetically modifying the coat protein prior to RNA assembly to present a reactive residue or targeting moiety.

Clause 20. The ring-shaped or halo-shaped nanoparticle of any one of clauses 1-19, wherein the nanoparticle comprises a surface-conjugated targeting moiety.

Clause 21. The ring-shaped or halo-shaped nanoparticle of clause 20, wherein the targeting moiety is an RGD peptide, antibody, or aptamer.

Clause 22. The ring-shaped or halo-shaped nanoparticle of any one clauses 1-21, wherein the RNA cargo encodes a cytokine selected from GM-CSF, IL-2, IL-10, or TNFα.

Clause 23. A composition comprising the ring-shaped or halo-shaped nanoparticle of any of clauses 1-17, and a carrier, and further optionally comprising an additional therapeutic agent, and further optionally wherein the additional therapeutic agent is encapsulated within a cavity of the ring-shaped nanoparticle, and further optionally wherein the additional therapeutic agent is an anti-cancer therapy.

Clause 24. A method for delivering a circRNA to a cell comprising contacting the cell with the ring-shaped or halo-shaped nanoparticle of any one of clauses 1-22, or the composition of clause 23.

Clause 25. The method of clause 24, wherein the contacting is in vivo or in vitro.

Clause 26. A method for treating or preventing cancer in a subject in need thereof comprising administering the ring-shaped or halo-shaped nanoparticle of any of clauses 1-22 or the composition of clause 23, optionally wherein the method wherein the nanoparticle further comprises an anti-cancer agent, thereby treating or preventing cancer into the subject.

Clause 27. The method of clause 26, wherein the cancer is selected from a blood cancer, a carcinoma or a sarcoma.

Clause 28. The method of clause 26 or 27, wherein the subject is a mammal, optionally wherein the mammal is selected from a human, an ape, a gibbon, a chimpanzee, an orangutan, a monkey, a macaques, a dog, a cat, a horse, a cow, a goat, a sheep, a pig, a mouse, a rabbit, or a guinea pig.

Clause 29. A method for manufacturing the nanoparticle of any one of clauses 1-22 comprising: (a) inoculating a plant with a viral vector encoding the filamentous virus, wherein the inoculation is by manual rubbing, mechanical inoculation, or foliar spray; (b) propagating and harvesting virus particles; (c) isolating coat protein subunits by disassembly; (d) contacting the coat protein subunits with an RNA cargo under assembly conditions to form the nanoparticle.

Clause 30. The method of clause 29, wherein the plant is Nicotiana benthamiana.

Clause 31. The method of clause 23 or 24, wherein the coat protein is recombinantly expressed in E. coli, yeast, or another heterologous host, optionally comprising an N-terminal histidine tag.

Clause 32. The method of any one of clauses 29-31, further comprising chemically or genetically modifying the coat protein prior to RNA assembly to present a reactive residue or targeting moiety.

Clause 33. A method for determining the mechanism of cellular uptake of the nanoparticle of any one of clauses 15-22 comprising contacting the nanoparticle with a cell in the presence of one or more endocytosis pathway inhibitors and determining uptake efficiency relative to a control.

Clause 34. The method of clause 33, wherein the inhibitors block clathrin-mediated endocytosis, caveolin-mediated endocytosis, or macropinocytosis.

Clause 35. A polynucleotide comprising: (a) a RNA polymerase promoter; (b) a polynucleotide encoding a filamentous plant virus coat protein, optionally wherein the filamentous plant virus coat protein is from Tobacco Mosaic Virus (TMV), Potato Virus X (PVX), Alternanthera mosaic virus, Papaya mosaic virus, or another member of the Potexvirus or Tobamovirus genera, optionally a potato virus X (PVX) genome or a fragment thereof, wherein the polynucleotide comprises an origin of assembly site sequence having a cis-acting element from the 5′ region of, and (c) a polyadenylation signal downstream of the polynucleotide encoding the plant virus coat protein or fragment.

Clause 36. The plasmid of clause 35, wherein the RNA polymerase promoter comprises a T7 promoter and/or the polyadenylation signal comprises a bovine growth hormone polyadenylation signal.

Clause 37. An in vitro transcribed RNA comprising: (a) a 5′ cap structure comprising 7-methylguanylate; (b) a polynucleotide encoding a filamentous plant virus coat protein, optionally wherein the filamentous plant virus coat protein is from Tobacco Mosaic Virus (TMV), Potato Virus X (PVX), Alternanthera mosaic virus, Papaya mosaic virus, or another member of the Potexvirus or Tobamovirus genera, optionally a potato virus X (PVX) genome or a fragment thereof having an origin of assembly site sequence having a cis-acting element from the 5′ region of origin of assembly site sequence; and (c) a 3′ polyadenylate tail, wherein the polynucleotide encoding the filamentous plant genome or fragment has a predetermined length selected to control the size of a virus-like particle assembled from the RNA.

Clause 38. A method of controlling the size of virus-like particles assembled from RNA derived from a plant virus genome, comprising generating RNA transcripts from a cloning vector that comprises a promoter for in vitro transcription, an origin of assembly site sequence from said plant virus genome, and a polyadenylation signal, wherein the RNA transcript is capped and polyadenylated, and wherein the length of the plant virus genome sequence determines the particle size.

### REFERENCES

1. Huang, X., Kon, E., Han, X., Zhang, X., Kong, N., Mitchell, M. J., Peer, D., & Tao, W. (2022). Nanotechnology-based strategies against SARS-CoV-2 variants. Nature nanotechnology, 17(10), 1027-1037. https://doi.org/10.1038/s41565-022-01174-5
2. Qin, S., Tang, X., Chen, Y., Chen, K., Fan, N., Xiao, W., Zheng, Q., Li, G., Teng, Y., Wu, M., & Song, X. (2022). mRNA-based therapeutics: powerful and versatile tools to combat diseases. Signal transduction and targeted therapy, 7(1), 166. https://doi.org/10.1038/s41392-022-01007-w
3. Rohner, E., Yang, R., Foo, K. S., Goedel, A., & Chien, K. R. (2022). Unlocking the promise of mRNA therapeutics. Nature biotechnology, 40(11), 1586-1600. https://doi.org/10.1038/s41587-022-01491-z
4. Hwang, H. J., & Kim, Y. K. (2024). Molecular mechanisms of circular RNA translation. Experimental & molecular medicine, 56(6), 1272-1280. https://doi.org/10.1038/s12276-024-01220-3
5. Azizgolshani, O., Garmann, R. F., Cadena-Nava, R., Knobler, C. M., & Gelbart, W. M. (2013). Reconstituted plant viral capsids can release genes to mammalian cells. Virology, 441(1), 12-17. https://doi.org/10.1016/j.virol.2013.03.001
6. Lam, P., & Steinmetz, N. F. (2018). Plant viral and bacteriophage delivery of nucleic acid therapeutics. Wiley interdisciplinary reviews. Nanomedicine and nanobiotechnology, 10(1), 10.1002/wnan.1487. https://doi.org/10.1002/wnan.1487
7. Kwon, S. J., Park, M. R., Kim, K. W., Plante, C. A., Hemenway, C. L., & Kim, K. H. (2005). cis-Acting sequences required for coat protein binding and in vitro assembly of Potato virus X. Virology, 334(1), 83-97. https://doi.org/10.1016/j.virol.2005.01.018
8. Thi, T. T. H., Suys, E. J. A., Lee, J. S., Nguyen, D. H., Park, K. D., & Truong, N. P. (2021). Lipid-Based Nanoparticles in the Clinic and Clinical Trials: From Cancer Nanomedicine to COVID-19 Vaccines. Vaccines, 9(4), 359. https://doi.org/10.3390/vaccines9040359
9. Waheed, I., Ali, A., Tabassum, H., Khatoon, N., Lai, W. F., & Zhou, X. (2024). Lipid-based nanoparticles as drug delivery carriers for cancer therapy. Frontiers in oncology, 14, 1296091. https://doi.org/10.3389/fonc.2024.1296091
10. Young, R. E., Hofbauer, S. I., & Riley, R. S. (2022). Overcoming the challenge of long-term storage of mRNA-lipid nanoparticle vaccines. Molecular therapy: the journal of the American Society of Gene Therapy, 30(5), 1792-1793. https://doi.org/10.1016/j.ymthe.2022.04.004
11. Hou, X., Zaks, T., Langer, R., & Dong, Y. (2021). Lipid nanoparticles for mRNA delivery. Nature reviews. Materials, 6(12), 1078-1094. https://doi.org/10.1038/s41578-021-00358-0
12. Johansson, J. M., Du Rietz, H., Hedlund, H., Eriksson, H. C., Oude Blenke, E., Pote, A., Harun, S., Nordenfelt, P., Lindfors, L., & Wittrup, A. (2025). Cellular and biophysical barriers to lipid nanoparticle mediated delivery of RNA to the cytosol. Nature communications, 16(1), 5354. https://doi.org/10.1038/s41467-025-60959-z
13. Zhang, T., Yin, H., Li, Y., Yang, H., Ge, K., Zhang, J., Yuan, Q., Dai, X., Naeem, A., Weng, Y., Huang, Y., & Liang, X. J. (2024). Optimized lipid nanoparticles (LNPs) for organ-selective nucleic acids delivery in vivo. iScience, 27(6), 109804. https://doi.org/10.1016/j.isci.2024.109804
14. Anguela, X. M., & High, K. A. (2019). Entering the Modern Era of Gene Therapy. Annual review of medicine, 70, 273-288. https://doi.org/10.1146/annurev-med-012017-043332
15. Chan, S. K., & Steinmetz, N. F. (2023). microRNA-181a silencing by antisense oligonucleotides delivered by virus-like particles. Journal of materials chemistry. B, 11(4), 816-825. https://doi.org/10.1039/d2tb02199d
16. Karan, S., Durán-Meza, A. L., Chapman, A., Tanimoto, C., Chan, S. K., Knobler, C. M., Gelbart, W. M., & Steinmetz, N. F. (2024). In Vivo Delivery of Spherical and Cylindrical In Vitro Reconstituted Virus-like Particles Containing the Same Self-Amplifying mRNA. Molecular pharmaceutics, 21(6), 2727-2739. https://doi.org/10.1021/acs.molpharmaceut.3c01105
17. Grinzato, A., Kandiah, E., Lico, C., Betti, C., Baschieri, S., & Zanotti, G. (2020). Atomic structure of potato virus X, the prototype of the Alphaflexiviridae family. Nature chemical biology, 16(5), 564-569. https://doi.org/10.1038/s41589-020-0502-4
18. Lee, K. L., Uhde-Holzem, K., Fischer, R., Commandeur, U., & Steinmetz, N. F. (2014). Genetic engineering and chemical conjugation of potato virus X. Methods in molecular biology (Clifton, N.J.), 1108, 3-21. https://doi.org/10.1007/978-1-62703-751-8_1
19. Verchot J. (2022). Potato virus X: A global potato-infecting virus and type member of the Potexvirus genus. Molecular plant pathology, 23(3), 315-320. https://doi.org/10.1111/mpp.13163
20. Atabekov, J., Dobrov, E., Karpova, O., & Rodionova, N. (2007). Potato virus X: structure, disassembly and reconstitution. Molecular plant pathology, 8(5), 667-675. https://doi.org/10.1111/j.1364-3703.2007.00420.x
21. Le, D. H., Lee, K. L., Shukla, S., Commandeur, U., & Steinmetz, N. F. (2017). Potato virus X, a filamentous plant viral nanoparticle for doxorubicin delivery in cancer therapy. Nanoscale, 9(6), 2348-2357. https://doi.org/10.1039/c6nr09099k
22. Comellas-Aragones, M., Engelkamp, H., Claessen, V. I., Sommerdijk, N. A., Rowan, A. E., Christianen, P. C., Maan, J. C., Verduin, B. J., Cornelissen, J. J., & Nolte, R. J. (2007). A virus-based single-enzyme nanoreactor. Nature nanotechnology, 2(10), 635-639. https://doi.org/10.1038/nnano.2007.299
23. Röder, J., Fischer, R., & Commandeur, U. (2017). Engineering Potato Virus X Particles for a Covalent Protein Based Attachment of Enzymes. Small (Weinheim an der Bergstrasse, Germany), 13(48), 10.1002/smll.201702151. https://doi.org/10.1002/smll.201702151
24. Schuphan, J., Stojanovid, N., Lin, Y. Y., Buhl, E. M., Aveic, S., Commandeur, U., Schillberg, S., & Fischer, H. (2024). A Combination of Flexible Modified Plant Virus Nanoparticles Enables Additive Effects Resulting in Improved Osteogenesis. Advanced healthcare materials, 13(16), e2304243. https://doi.org/10.1002/adhm.202304243
25. Jobsri, J., Allen, A., Rajagopal, D., Shipton, M., Kanyuka, K., Lomonossoff, G. P., Ottensmeier, C., Diebold, S. S., Stevenson, F. K., & Savelyeva, N. (2015). Plant virus particles carrying tumour antigen activate TLR7 and Induce high levels of protective antibody. PloS one, 10(2), e0118096. https://doi.org/10.1371/journal.pone.0118096
26. Zu, H., & Gao, D. (2021). Non-viral Vectors in Gene Therapy: Recent Development, Challenges, and Prospects. The AAPS journal, 23(4), 78. https://doi.org/10.1208/s12248-021-00608-7
27. Daniell, H., Streatfield, S. J., & Rybicki, E. P. (2015). Advances in molecular farming: key technologies, scaled up production and lead targets. Plant biotechnology journal, 13(8), 1011-1012. https://doi.org/10.1111/pbi.12478
28. Tsekoa, T. L., Singh, A. A., & Buthelezi, S. G. (2020). Molecular farming for therapies and vaccines in Africa. Current opinion in biotechnology, 61, 89-95. https://doi.org/10.1016/j.copbio.2019.11.005
29. Baiersdorfer, M., Boros, G., Muramatsu, H., Mahiny, A., Vlatkovic, I., Sahin, U., & Karikó, K. (2019). A Facile Method for the Removal of dsRNA Contaminant from In Vitro-Transcribed mRNA. Molecular therapy. Nucleic acids, 15, 26-35. https://doi.org/10.1016/j.omtn.2019.02.018
30. Carette, N., Engelkamp, H., Akpa, E., Pierre, S. J., Cameron, N. R., Christianen, P. C., Maan, J. C., Thies, J. C., Weberskirch, R., Rowan, A. E., Nolte, R. J., Michon, T., & Van Hest, J. C. (2007). A virus-based biocatalyst. Nature nanotechnology, 2(4), 226-229. https://doi.org/10.1038/nnano.2007.76
31. Schuphan, J., Stojanovid, N., Lin, Y. Y., Buhl, E. M., Aveic, S., Commandeur, U., Schillberg, S., & Fischer, H. (2024). A Combination of Flexible Modified Plant Virus Nanoparticles Enables Additive Effects Resulting in Improved Osteogenesis. Advanced healthcare materials, 13(16), e2304243. https://doi.org/10.1002/adhm.202304243
32. Le, D. H. T., Commandeur, U., & Steinmetz, N. F. (2019). Presentation and Delivery of Tumor Necrosis Factor-Related Apoptosis-Inducing Ligand via Elongated Plant Viral Nanoparticle Enhances Antitumor Efficacy. ACS nano, 13(2), 2501-2510. https://doi.org/10.1021/acsnano.8b09462
33. Tong, M., Palmer, N., Dailamy, A., Kumar, A., Khaliq, H., Han, S., Finburgh, E., Wing, M., Hong, C., Xiang, Y., Miyasaki, K., Portell, A., Rainaldi, J., Suhardjo, A., Nourreddine, S., Chew, W. L., Kwon, E. J., & Mali, P. (2025). Robust genome and cell engineering via in vitro and in situ circularized RNAs. Nature biomedical engineering, 9(1), 109-126. https://doi.org/10.1038/s41551-024-01245-z
34. McNeil, B. A., Simon, D. M., & Zimmerly, S. (2014). Alternative splicing of a group II intron in a surface layer protein gene in Clostridium tetani. Nucleic acids research, 42(3), 1959-1969. https://doi.org/10.1093/nar/gkt1053
35. Pyle A. M. (2016). Group II Intron Self-Splicing. Annual review of biophysics, 45, 183-205. https://doi.org/10.1146/annurev-biophys-062215-011149
36. Zimmerly, S., & Semper, C. (2015). Evolution of group II introns. Mobile DNA, 6, 7. https://doi.org/10.1186/s13100-015-0037-5
37. Balke, I., & Zeltins, A. (2019). Use of plant viruses and virus-like particles for the creation of novel vaccines. Advanced drug delivery reviews, 145, 119-129. https://doi.org/10.1016/j.addr.2018.08.007
38. Wang, C., Fernindez de Ávila, B. E., Mundaca-Uribe, R., Lopez-Ramirez, M. A., Ramirez-Herrera, D. E., Shukla, S., Steinmetz, N. F., & Wang, J. (2020). Active Delivery of VLPs Promotes Anti-Tumor Activity in a Mouse Ovarian Tumor Model. Small (Weinheim an der Bergstrasse, Germany), 16(20), e1907150. https://doi.org/10.1002/smll.201907150
39. Chung, Y. H., Ortega-Rivera, O. A., Volckaert, B. A., Jung, E., Zhao, Z., & Steinmetz, N. F. (2023). Viral nanoparticle vaccines against S100A9 reduce lung tumor seeding and metastasis. Proceedings of the National Academy of Sciences of the United States of America, 120(43), e2221859120. https://doi.org/10.1073/pnas.2221859120
40. Lacasse, P., Denis, J., Lapointe, R., Leclerc, D., & Lamarre, A. (2008). Novel plant virus-based vaccine induces protective cytotoxic T-lymphocyte-mediated antiviral immunity through dendritic cell maturation. Journal of virology, 82(2), 785-794. https://doi.org/10.1128/JVI.01811-07
41. Shukla, S., Roe, A. J., Liu, R., Veliz, F. A., Commandeur, U., Wald, D. N., & Steinmetz, N. F., (2020). Affinity of plant viral nanoparticle potato virus X (PVX) towards malignant B cells enables cancer drug delivery. Biomaterials science, 8(14), 3935-3943. https://doi.org/10.1039/d0bm00683a
42. a, X. M., & High, K. A. (2019). Entering the Modern Era of Gene Therapy. Annual review of medicine, 70, 273-288. https://doi.org/10.1146/annurev-med-012017-043332

What is claimed is:

1. A ring-shaped or halo-shaped nanoparticle comprising a filamentous plant virus coat protein, optionally wherein the filamentous plant virus coat protein is from Tobacco Mosaic Virus (TMV), Potato Virus X (PVX), Alternanthera mosaic virus, Papaya mosaic virus, or another member of the Potexvirus or Tobamovirus genera, the coat protein being assembled on one or more circular RNA (circRNA), optionally wherein the one or more circRNA are the same or different from each other.

2. The ring-shaped or halo-shaped nanoparticle of claim 1, wherein the filamentous plant virus coat protein is from Tobacco Mosaic Virus (TMV) or Potato Virus X (PVX).

3. The ring-shaped or halo-shaped nanoparticle of claim 1, wherein the coat protein comprises chemically modified lysine, cysteine, or histidine residues to permit conjugation of a therapeutic, diagnostic, or targeting moiety.

4. The ring-shaped or halo-shaped nanoparticle of claim 1, wherein the targeting moiety is selected from an RGD peptide, an antibody or antibody fragment, an aptamer, a ligand for a cell-surface receptor, or a carbohydrate.

5. The ring-shaped or halo-shaped nanoparticle of claim 1, wherein the RNA cargo is a self-amplifying RNA (replicon) encoding an RNA-dependent RNA polymerase (RdRp) and a gene of interest separated by a self-cleaving peptide sequence.

6. The ring-shaped nanoparticle of claim 5, wherein the RNA-dependent RNA polymerase is derived from an alphavirus or a nodavirus.

7. The ring-shaped nanoparticle of claim 3, wherein the coat protein subunits are crosslinked with a bifunctional or multifunctional crosslinker, optionally a polyethylene glycol (PEG) crosslinker and/or comprises a membrane-active, pH-sensitive fusogenic peptide.

8. The ring-shaped or halo-shaped nanoparticle of claim 1, further comprising a circRNA cassette comprising the one or more circRNA, optionally wherein the circRNA cassette comprises an internal ribosome entry site (IRES), optionally further comprising an origin of assembly site (OAS), and optionally further comprising a RNA-dependent RNA polymerase (RdRp).

9. A composition comprising the ring-shaped or halo-shaped nanoparticle of claim 1, and a carrier, and further optionally comprising an additional therapeutic agent, and further optionally wherein the additional therapeutic agent is encapsulated within a cavity of the ring-shaped nanoparticle, and further optionally wherein the additional therapeutic agent is an anti-cancer therapy.

10. A method for delivering a circRNA to a cell comprising contacting the cell with the ring-shaped or halo-shaped nanoparticle of claim 1, optionally wherein the contacting is in vivo or in vitro.

11. A method for treating or preventing cancer in a subject in need thereof comprising administering the ring-shaped or halo-shaped nanoparticle of claim 1, wherein the nanoparticle further comprises an anti-cancer agent, thereby treating or preventing cancer into the subject.

12. A method for manufacturing the nanoparticle of claim 1 comprising:
(a) inoculating a plant with a viral vector encoding the filamentous virus, wherein the inoculation is by manual rubbing, mechanical inoculation, or foliar spray;
(b) propagating and harvesting virus particles;
(c) isolating coat protein subunits by disassembly;
(d) contacting the coat protein subunits with an RNA cargo under assembly conditions to form the nanoparticle.

13. A polynucleotide comprising:
(a) a RNA polymerase promoter;
(b) a polynucleotide encoding a filamentous plant virus coat protein, optionally wherein the filamentous plant virus coat protein is from Tobacco Mosaic Virus (TMV), Potato Virus X (PVX), Alternanthera mosaic virus, Papaya mosaic virus, or another member of the Potexvirus or Tobamovirus genera, optionally a potato virus X (PVX) genome or a fragment thereof, wherein the polynucleotide comprises an origin of assembly site sequence having a cis-acting element from the 5′ region of, and
(c) a polyadenylation signal downstream of the polynucleotide encoding the plant virus coat protein or fragment.

14. The polynucleotide of claim 13, wherein the RNA polymerase promoter comprises a T7 promoter and/or the polyadenylation signal comprises a bovine growth hormone polyadenylation signal.

15. A vector comprising the polynucleotide of claim 13, wherein the vector is a plasmid, cosmid, bacterial artificial chromosome, yeast artificial chromosome, or viral vector, and optionally wherein the vector is a plasmid selected from pCMV-T7-SL1-EGFP, Circ-EMCV-GFP-SL1, pCMV-T7-PVX-Genome, or pHGWA-9×His-PVX-CP, or a derivative or equivalent thereof.

16. The vector of claim 15, wherein the vector further comprises a selectable marker, an origin of replication, and optionally one or more regulatory sequences operably linked to the polynucleotide.

17. The vector of claim 15, wherein the vector is configured for expression in a plant cell, bacterial cell, yeast cell, or mammalian cell.

18. A method for in vitro transcription of RNA for assembly into a virus-like particle or the ring-shaped nanoparticle comprising expressing in vitro the polynucleotide of claim 13.

19. An in vitro transcribed RNA comprising:
(a) a 5′ cap structure comprising 7-methylguanylate;
(b) a polynucleotide encoding a filamentous plant virus coat protein, optionally wherein the filamentous plant virus coat protein is from Tobacco Mosaic Virus (TMV), Potato Virus X (PVX), Alternanthera mosaic virus, Papaya mosaic virus, or another member of the Potexvirus or Tobamovirus genera, optionally a potato virus X (PVX) genome or a fragment thereof having an origin of assembly site sequence having a cis-acting element from the 5′ region of origin of assembly site sequence; and
(c) a 3′ polyadenylate tail, wherein the polynucleotide encoding the filamentous plant genome or fragment has a predetermined length selected to control the size of a virus-like particle assembled from the RNA.

20. A method of controlling the size of virus-like particles assembled from RNA derived from a plant virus genome, comprising generating RNA transcripts from a cloning vector that comprises a promoter for in vitro transcription, an origin of assembly site sequence from said plant virus genome, and a polyadenylation signal, wherein the RNA transcript is capped and polyadenylated, and wherein the length of the plant virus genome sequence determines the particle size.
